(19) United States
(12) Patent Application Publication
(10) Pub. No.: US 2026/0284002 A1
(43) Pub. Date: Sep. 24, 2026

(54) COMPOSITIONS AND METHODS OF CONTROLLING MAMMALIAN SATIETY

(71) Applicant: VitaKey Inc., Washington, DC (US)

(72) Inventors: Aaron C. Anselmo, Chapel Hill, NC (US); Anant S. Balijepalli, Durham, NC (US); Emily E. Bonacquisti, Chapel Hill, NC (US); Andrew D. Christenson, Raleigh, NC (US); Joshua K. DeGennaro, Cary, NC (US); Seo Yeon Kim, Apex, NC (US); Andrea Stamp, Brookline, MA (US); Ana Jaklenec, Lexington, MA (US); Catherine B. Reynolds, Mountain Brook, AL (US); Robert S. Langer, Newton, MA (US)

(21) Appl. No.: 19/474,473

(22) PCT Filed: Apr. 12, 2024
(86) PCT No.: PCT/US2024/024517

§ 371 Date: Oct. 10, 2025

Related U.S. Application Data
(60) Provisional application No. 63/459,575, filed on Apr. 14, 2023.
(60) Provisional application No. 63/517,291, filed on Aug. 2, 2023.
(60) Provisional application No. 63/579,919, filed on Aug. 31, 2023.
(60) Provisional application No. 63/617,747, filed on Jan. 4, 2024.

(51) Int. Cl. A61K 31/352; A23C 9/13; A61K 9/00; A61K 9/50; A61K 31/201; A61K 31/202; A61K 31/575; A61K 31/7004; A61K 31/717; A61K 31/718; A61K 31/733; A61K 38/17

(52) U.S. Cl. CPC A61K 31/352; A23C 9/1307; A23C 9/1315; A23C 9/1322; A61K 9/0056; A61K 9/0095; A61K 9/5073; A61K 9/5089; A61K 31/201; A61K 31/202; A61K 31/575; A61K 31/7004; A61K 31/717; A61K 31/718; A61K 31/733; A61K 38/1709

(57) ABSTRACT

A particle preparation further characterized as a core-shell preparation comprising a core component and at least two shell component(s). The particle preparation comprises at least one lipid, at least one protein, at least one carbohydrate, and at least one acaloric phytonutrient. A method of administering particle preparations to one or more mammal(s) so as to control satiety.

### I. CROSS-REFERENCE TO RELATED APPLICATION

The present application claims priority to U.S. Provisional Application No. 63/459,575, filed Apr. 14, 2023, U.S. Provisional Application No. 63/517,291, filed Aug. 2, 2023, U.S. Provisional Application No. 63/579,919, filed Aug. 31, 2023, and U.S. Provisional Application No. 63/617,747, filed Jan. 4, 2024, the entireties of which are incorporated herein by reference.

### II. TECHNICAL FIELD

The present disclosure is generally related to food and/or beverage compositions (e.g., formulated meals, formulated foods, formulated beverages, formulated supplements, and/or their nutritional constituents, etc.; e.g., formulated ingestibles) and technologies (e.g., methods of preparation, use, etc.) relating thereto.

### III. BACKGROUND

The health benefits of formulated meals, formulated foods, formulated beverages, formulated supplements, and/or their nutritional constituents to mammals, such as humans, have been studied for several decades. However, in many cases, the challenges associated with controlled delivery of nutrients and the subsequent control over the benefits of nutrients (e.g., satiety, energy acquisition, etc.) from formulated meals, formulated foods, formulated beverages, formulated supplements, and/or their nutritional constituents remain unsolved. As such, meals, foods, beverages, supplements, and/or their nutritional constituents have yet to realize their full potential in controlling the downstream benefits (e.g., satiety, energy acquisition, etc.) of nutrients.

### IV. SUMMARY

The present disclosure is generally related to satiety modulating composition(s) (e.g., formulated satiety modulator(s)) and technologies (e.g., methods of use.) relating thereto. The present disclosure is generally related to method(s) of controlling the satiety of one or more mammal(s) (e.g., method(s) of administering one or more satiety modulating composition(s)). In certain embodiments, satiety modulating composition(s) are or may be orally administered alone and/or included in a supplement, a food, a supplemented (i.e., fortified) food product, a beverage, a supplemented (i.e., fortified) beverage product, a powder, or a supplemented (i.e., fortified) powder product intended to confer health benefits and/or control satiety and/or control cravings. In certain embodiments, satiety modulating composition(s) are or may be administered (e.g., orally, parenterally [e.g., intramuscularly, intravenously, percutaneously, subcutaneously, etc.], intranasally, etc) administered alone; in certain embodiments, a satiety modulating composition may be included in a format (e.g., a pharmaceutical format) such as a capsule, device, elixir, solution, suspension, suppository, syrup, tablet, et.

Satiety modulating composition(s) known to those of ordinary skill in the art comprise formulated therapeutic entities such as, for example, small molecules and/or peptides. Satiety modulating composition(s) known to those skilled in the art comprise formulated food ingredients including formulated carbohydrates, fats, and/or peptides. Those satiety modulating composition(s) utilizing formulated therapeutic peptide(s), substantially containing exogenous incretin mimics demonstrate increased satiety and are widely accepted. Similarly, satiety modulating composition(s) utilizing therapeutic small molecule(s) demonstrate increased satiety and are on the cusp of regulatory approval. Attempts to modulate satiety that aim to access incretin hormonal mechanisms via natural (i.e., not synthesized wholly, or in part, from at least one unnatural chemical compound) food ingredients, however, have failed to demonstrate efficacy, at least in part due to challenges in identifying optimal non-therapeutic satiety modulator(s), ratios of satiety modulator(s), spatial arrangement of satiety modulator(s), and/or delivering satiety modulator(s) to their optimal site of action. As a result, identification of satiety modulating composition(s) remains difficult.

Methods of controlling the satiety of one or more mammal(s) known to those of ordinary skill in the art comprise administration of one or more formulated therapeutic small molecules and/or peptides. Methods of controlling the satiety of one or more mammal(s) known to those skilled in the art comprise administration of one or more formulated non-therapeutic carbohydrates, fats, and/or peptides. Methods of controlling the satiety of one or more mammal(s) comprising administration of formulated peptide(s), substantially containing therapeutic, exogenous semi-synthetic incretins, demonstrate increased satiety and are widely accepted. These methods of controlling satiety, however, require an essential step of frequent subcutaneous dosing associated with painful administration and reduced patient compliance. Other methods of controlling the satiety of one or more mammal(s) comprising a step of administering satiety modulating composition(s) comprising food ingredient(s) fail to demonstrate efficacy due to poorly optimized satiety modulating composition(s) for oral delivery. As a result, methods of controlling the satiety of one or more mammal(s) remain difficult.

Moreover, methods of controlling the satiety of one or more mammal(s) relying upon a step of oral administration of one or more non-therapeutic satiety modulating composition(s) known to those skilled in the art fail to account for spatial targeting of one or more satiety modulator(s). Without wishing to be bound by any particular theory, it is contemplated that leveraging endogenous gut hormone secretion requires careful spatial targeting to one or more biological compartments (e.g., mouth, esophagus, stomach, duodenum, jejunum, ileum, cecum, colon, rectum). Moreover, oral administration of one or more satiety modulating composition(s) known to those skilled in the art fail to account for an integration of satiety response(s) (e.g., simultaneous secretion of several satiety hormones). For example, herein are disclosed satiety modulating composition(s) characterized as releasing one or more lipid(s) in both upper and lower gastrointestinal tracts unknown to those skilled in the art. Without wishing to be bound by any particular theory, it is contemplated that effective non-therapeutic satiety modulating composition(s) influence the transcription, translation, post-translational modification, secretion, and/or metabolism of at least one satiety marker (e.g., GLP-1, GLP-2, GIP, PYY, CCK, somatostatin, motilin, amylin, etc.). Without wishing to be bound by any particular theory, the release of satiety modulator(s) in both upper and lower gastrointestinal tracts is of particular novelty so as to maximally influence the transcription, translation, post-translational modification, secretion, and/or metabolism of at least one satiety marker. As a result, identifying methods of controlling the satiety of one or more mammal(s) via satiety modulating compositions remains difficult.

Current efforts to control (e.g., modulate) satiety of mammals (e.g., humans) are limited by the development and implementation of non-therapeutic satiety modulating composition(s) (e.g., formulated satiety modulator(s)) that are compatible with various food, beverage, supplement, and/or dosage format(s). For example, in some non-limiting instances, one or more satiety modulator(s) further characterized as partial agonists of GPR40 (e.g., linoleic acid, oleic acid) are or may be liquid at least at 20° C., 25° C., 30° C., 35° C., and/or 37° C. Without wishing to be bound by any particular theory, it is contemplated that satiety modulator(s) and/or satiety modulating composition(s) further characterized as liquids present challenges relating to, for example, spatial arrangement and coating (e.g., formulation). Without wishing to be bound by any particular theory, it is contemplated that satiety modulator(s) and/or satiety modulating composition(s) further characterized as liquids present challenges relating to, for example, stability when introduced into one or more food and/or beverage product(s) and/or stability upon oral administration. As such, identification of satiety modulating composition(s) remains difficult.

The present disclosure provides certain insights towards preparing satiety modulating composition(s) comprising effective satiety modulator(s), effective ratios of combination(s) of satiety modulator(s), effective spatial arrangement of satiety modulator(s), and/or effective spatiotemporal targeting of delivery of satiety modulator(s). The present disclosure provides for one or more satiety modulating composition(s) characterized as being compatible with various food, beverage, supplement, and/or dosage format(s) intended to confer health benefits, satiety, and/or cravings. Provided insights are or may be particularly useful towards the fortification (e.g., supplementation) of one or more ingestible product(s) as described herein.

In some embodiments, the present disclosure provides technologies (e.g., satiety modulating compositions) comprising one or more non-therapeutic satiety modulator(s). In some non-limiting instances, one or more satiety modulator(s) is or may be characterized as a lipid, a protein, (e.g., amino acids, peptides, polypeptides, proteins), a carbohydrate (e.g., mono-, di-, oligo-, poly-saccharides, fiber), an acaloric phytonutrient, and/or combinations thereof. In some non-limiting embodiments, one or more satiety modulator(s) as provided herein are or may be effective, either alone or in combination, in stimulating the secretion of GLP-1, GLP-2, GIP, PYY, CCK, somatostatin, motilin, amylin, etc. In some embodiments, the present disclosure provides technologies (e.g., satiety modulating compositions) comprising one or more satiety modulator(s) further characterized as structure-modifying component(s). In some non-limiting instances, one or more structure-modifying component(s) is or may be characterized as a lipid, a protein (e.g., amino acids, peptides, polypeptides, proteins, etc.), a carbohydrate (e.g., mono-, di-, oligo-, poly-saccharides, fiber, etc.), an acaloric phytonutrient, and/or combinations thereof. Without wishing to be bound by any particular theory, it is contemplated that one or more structure-modifying component(s) improves the stability of one or more satiety modulating composition(s) in food and/or beverage products, during food processing, and/or during administration while additionally providing for satiety modulation. Without wishing to be bound by any particular theory, it is contemplated that one or more structure-modifying component(s) confers solidification, resistance to water, resistance to oxygen, resistance to acid, resistance to enzymatic degradation, and/or increased residence time within a mammal.

In some embodiments, the present disclosure provides technologies (e.g., satiety modulating compositions) further characterized as particle preparation(s). In some non-limiting instances, one or more particle preparation(s) confers spatiotemporal targeting by preventing release of core component(s). In some non-limiting instances, one or more particle preparation(s) confers spatiotemporal targeting by enabling immediate release of shell component(s). In some non-limiting instances, one or more satiety modulating composition(s) are characterized as particle preparation(s) wherein the release of an inner shell component is controlled by an outer shell component. In some preferred instances, core component(s) and shell component(s) are characterized as satiety modulator(s) (e.g., lipids, proteins, carbohydrates, and acaloric phytonutrients, as provided herein).

Without wishing to be bound by any particular theory, it is contemplated that one or more shell component(s) are useful to provide for delivery of one or more satiety modulator(s) to a specific gastrointestinal region (e.g., stomach, duodenum, jejunum, ileum, cecum, colon, Peyer's patches, etc.), to specific cell types (e.g., enterocytes, Paneth cells, goblet cells, M cells, L cells, etc.), with specified residence time, and/or with increased or decreased bioavailability. For example, in some preferred instances, one or more shell component(s) are useful to provide for the delivery of distinct satiety modulator(s) to both the upper and lower gastrointestinal tract using a single satiety modulating composition.

In some embodiments, satiety compositions (e.g., macronutrients, formulated protein, formulated fat, formulated carbohydrate) provided by the present disclosure include a first layer that is or comprises a satiety modulator and/or a second satiety modulator and a second layer that is or comprises a different satiety modulator and/or the same satiety modulator. For example, in some particular embodiments, a satiety modulating material may be or comprise lipids and/or carbohydrates; in some embodiments, the lipid may be encapsulated within the carbohydrate; in some embodiments, the carbohydrate may be encapsulated within the lipid; in some embodiments, the carbohydrate may be encapsulated within the same or a distinct carbohydrate. In some embodiments, the carbohydrate may be encapsulated within a mixture of lipid and carbohydrates. In some embodiments, the layers are reversed.

In some embodiments, provided satiety compositions (e.g., formulated ingestible macronutrients) achieve one or more advantages for macronutrients/calorie sources/energy sources (e.g., proteins, carbohydrates, fats) such as delivery to specific parts of the gastrointestinal tract (e.g., stomach, small intestine, duodenum, jejunum, ileum, cecum, large intestine, Peyer's patches, etc.), delivery to specific cells in the gastrointestinal tract (e.g., enterocytes, Paneth cells, goblet cells, M cells, L cells, neuroendocrine cells, etc.), controlled residence time, controlled concentration of non-therapeutic satiety modulators in biological compartment of release, controlled binding and/or adsorption to cell receptors, controlled spatial distribution in the gastrointestinal tract, controlled coverage of the surface area in the gastrointestinal tract, controlled release, sustained release, on-demand bolus release, stimuli-responsive release, decreased and/or increased absorption after ingestion, increased bioavailability, controlled satiety, decreased gastrointestinal discomfort and compatibility with other materials.

In some embodiments, provided satiety compositions (e.g., macronutrients, formulated protein, formulated fat, formulated carbohydrate) are characterized by one or more of the following advantages:
a) Non-therapeutic satiety modulating composition(s);
b) Improved changes in circulating levels of several satiety hormone(s);
c) Satiety modulating composition(s) are primarily comprised of edible satiety modulator(s);
d) Satiety modulator(s) not known in the prior art;
e) Novel satiety modulator(s) enhancing the efficacy of known satiety modulator(s);
f) Core-shell structure(s) for targeting of multiple gastrointestinal region(s);
g) Improved delivery of payloads (e.g., macronutrients, ratios of proteins to fats to carbohydrates) to specific sites in the gastrointestinal tract (e.g., ileum);
h) Improved delivery of payloads (e.g., macronutrients, ratios of proteins, fats, carbohydrates) to specific cells in the gastrointestinal tract (e.g., L cells);
i) Improved delivery of a specific ratio of macronutrient payloads (e.g., ratios of proteins to fats to carbohydrates) that modulates satiety;
j) Improved delivery of a specific concentration of macronutrient payloads that modulates satiety;
k) Spatiotemporal control over the delivery of a specific ratio and/or a specific concentration of macronutrient payloads that modulates satiety;
l) Compatibility with and/or in food and/or food products, with and/or in beverages and/or beverage products, with and/or in supplements, with and/or in dry powders;
m) Improved shelf-life and resistance to degradation at/in:
   i) decreased temperatures (e.g., −80° C., −20° C., and/or 4° C.)
   ii) ambient temperatures (e.g., 10° C., 15° C., and/or 20° C.)
   iii) elevated temperatures (e.g., 22° C., 25° C., 30° C., 35° C., and/or 40° C.) iv) the presence of high relative humidity (e.g., up to 100%)
   v) the presence of moisture
   vi) the presence of oxygen
   vii) the presence of light
   viii) the presence of heat
   ix) the presence of surfactants
   x) liquids:
      (1) acidic liquids (pH values between 1-6)
      (2) neutral liquids (pH values between 6-8)
      (3) basic liquids (pH values between 8-12) xi) or a combination thereof;
   n) Prolonged residence time or transit time in the gastrointestinal tract or gastrointestinal tract compartments;
o) Controlled release or sustained release of payload components in the gastrointestinal tract;
p) Controlled spatial distribution of payloads in and/or on the gastrointestinal tract;
q) Controlled concentration of payloads in the gastrointestinal tract (e.g., in the stomach, in the intestines, at the epithelial surface, in the mucus, etc.);
r) Improved shelf-life in food or beverage matrices
   i) protein bars
   ii) dry powders
   iii) milk powders
   iv) whey powders
   v) yogurt
   vi) drinkable yogurt;
s) Improved payload resistance to losses during manufacturing processes:
   i) pasteurization
   ii) shear mixing
   iii) elevated pressurized processes
   iv) elevated temperature processes
   v) or combinations thereof;
t) Tunable properties including
   i) size
   ii) coating thickness
   iii) morphology
   iv) geometry
   v) loading
   vi) dose
   vii) interactions with the surrounding environment
   viii) release conditions
   ix) or combinations thereof;
u) Maintenance and preservation of composition morphology (e.g., particle geometry) when exposed to typically degrading conditions;
v) Changes in circulating satiety hormone concentrations by at least 20%; and
w) Satiety is controlled for at least 4 hours, at least 6 hours, at least 12 hours, and/or at least 24 hours.

In some embodiments, the present disclosure provides satiety composition(s) that are or comprise a particle preparation, wherein particles of the particle preparation comprise (i) shell component(s); and (ii) a core component(s), wherein the either component comprises satiety modulator(s) together compatible with supplement, food, beverage, and/or physiological fluid/environments (e.g., stomach acids, stomach, intestines, etc.). In certain preferred embodiments, shell component(s) achieve one or more of: (i) protection (maintenance/preservation of satiety modulator stability) of the payload in supplements, foods, beverages, and/or physiological fluids/environment (e.g., stomach acids, stomach, intestines, etc.), (ii) extending satiety modulator retention time, (iii) controlling satiety modulator spatial interactions within the host, (iv) controlling satiety modulator release rate, (v) increasing or decreasing satiety modulator absorption, (vi) increasing or decreasing satiety modulator concentrations within the host that confer satiety benefits, and/or (vii) release of satiety modulators in the upper gastrointestinal tract. In certain preferred embodiments, core component(s) achieve one or more of: (i) protection (maintenance/preservation of satiety modulator stability) of the payload in supplements, foods, beverages, and/or physiological fluids/environment (e.g., stomach acids, stomach, intestines, etc.), (ii) controlling satiety modulator release rate, (iii) increasing or decreasing satiety modulator absorption, (iv) increasing or decreasing satiety modulator concentrations within the host that confer satiety benefits, and/or (v) release of satiety modulators in the lower gastrointestinal tract.

As disclosed herein, provided satiety modulating composition(s) are essentially non-therapeutic (e.g., comprised of non-therapeutic components). As provided herein, non-therapeutic components are not approved by one or more governing bodies to treat, mitigate, cure, or address a disease suffered by one or more mammal(s). Several satiety modulating composition(s) known as prior art are essentially therapeutic, being comprised of synthetic compounds approved to treat, mitigate, cure, or address disease(s) suffered by one or more mammal(s). In an unexpected result, the non-therapeutic satiety modulating composition(s) provided herein are useful for improving the health of one or more mammal(s) despite a lack of regulatory approval.

As disclosed herein, provided satiety modulating composition(s) are characterized as efficacious to change circulating quantity (e.g., serum concentration(s)) of hormone(s) related to satiety in one or more mammal(s). Satiety modulating composition(s) known as prior art are each efficacious in changing the quantity of one satiety hormone (e.g., GLP-1 or PYY) upon administration to one or more mammal(s). In an unexpected result, the selection and structure of satiety modulating composition(s) provided herein, comprising one or more lipid(s), one or more protein(s), one or more carbohydrate(s), and/or one or more acaloric phytonutrient(s) are essentially efficacious to simultaneously change the circulating quantities of multiple satiety hormones.

As disclosed herein, provided satiety modulating composition(s) are comprised of one or more satiety modulator(s). Satiety modulating composition(s) known as prior art are characterized as efficacious due to the presence of satiety modulator(s) and inedible excipient compounds that enhance the activity of one or more satiety modulator(s). In an unexpected result, the selection and spatial arrangement of one or more satiety modulator(s) in provided satiety modulating composition(s) enables elimination of inedible excipient compounds. As such, provided satiety modulating composition(s) are of considerable novelty, comprised substantially of edible satiety modulator(s) retaining efficacy.

As disclosed herein, provided satiety modulating composition(s) are comprised of one or more satiety modulator(s) further characterized as a lipid. Satiety modulating composition(s) known as prior art comprising lipids suffer from lack of efficacy due to improper selection of lipid component(s). In an unexpected result, the selection of fatty amide lipid component(s) in provided satiety modulating composition(s) exhibits great efficacy in changing the circulating quantity of one or more satiety hormone(s). In an unexpected result, the selection of fatty amide lipid component(s) in the presence of alkene-containing fatty acid lipid component(s) is further efficacious in changing the circulating quantity of one or more satiety hormone(s).

As disclosed herein, provided satiety modulating composition(s) are comprised of one or more satiety modulator(s) further characterized as a lipid. Satiety modulating composition(s) known as prior art comprising lipids suffer from lack of efficacy due to improper selection of lipid component(s). In an unexpected result, the selection of sterol, stanol, terpene, and/or terpenoid lipid component(s) in provided satiety modulating composition(s) exhibits great efficacy in changing the circulating quantity of one or more satiety hormone(s). In an unexpected result, the selection of sterol, stanol, terpene, and/or terpenoid lipid component(s) in the presence of alkene-containing fatty acid lipid component(s) is further efficacious in changing the circulating quantity of one or more satiety hormone(s). Without wishing to be bound by any particular theory, it is contemplated that of sterol, stanol, terpene, and/or terpenoid lipid component(s) mitigate emulsification of alkene-containing fatty acids by bile salts to prevent absorption.

As disclosed herein, provided satiety modulating composition(s) are comprised of one or more satiety modulator(s) further characterized as an acaloric phytonutrient. The inclusion of an acaloric phytonutrient is unknown to the prior art in one or more satiety modulating composition(s). Without wishing to be bound by any particular theory, it is contemplated that one or more acaloric phytonutrients is an agonist, allosteric modulator, and/or inhibitor of nutrient sensors, nutrient transporters, and/or satiety hormone receptors present in the gastrointestinal tract. In an unexpected result, the inclusion of acaloric phytonutrient(s) is sufficient to confer an efficacy towards provided satiety modulating composition(s). In an unexpected result, the inclusion of acaloric phytonutrient(s) is sufficient to confer enhanced efficacy towards provided satiety modulating composition(s) further comprising an alkene-containing fatty acid.

As disclosed herein, provided satiety modulating composition(s) are characterized as particle preparation(s). Satiety modulating composition(s) characterized as particle preparation(s) known as prior art suffer from lack of resistance to pH greater than 4.5 and a lack of compatibility with food and/or beverage products. In an unexpected result, the provided particle preparation(s) comprising an inner shell with sensitivity to pH greater than 4.5 and an outer shell with sensitivity to pH less than 4.5 are compatible in food and/or beverage products and efficacious to change circulating quantity of satiety hormone(s).

As disclosed herein, provided satiety modulating composition(s) are characterized as particle preparation(s). Satiety modulating composition(s) characterized as particle preparation(s) known as prior art are characterized by a core of one or more satiety modulator(s) encapsulated in an inedible shell, thus suffering from targeting of a single gastrointestinal region. In an unexpected result, one or more satiety modulator(s) in provided satiety modulating composition(s) are incorporated into core component(s) and shell component(s), enabling targeting of several gastrointestinal regions.

As disclosed herein, provided satiety modulating composition(s) are characterized as particle preparation(s). Satiety modulating composition(s) characterized as particle preparation(s) known as prior art are characterized by poor resistance to water, thus reducing efficacy. In an unexpected result, one or more satiety modulator(s) present in one or more shell component(s) in provided satiety modulating composition(s) confer resistance to water and prolong the stability (vide infra) of provided satiety modulating composition(s).

As disclosed herein, provided satiety modulating composition(s) are characterized as particle preparation(s). Satiety modulating composition(s) characterized as particle preparation(s) known as prior art are characterized by rapid clearance in the gastrointestinal tract and short duration of action. In an unexpected result, one or more satiety modulator(s) present in one or more shell component(s) in provided satiety modulating composition(s) confer retention (vide infra) in the gastrointestinal tract to prolong the efficacy of provided satiety modulating composition(s). Without wishing to be bound by any particular theory, retention of one or more satiety modulating composition(s) is contemplated to occur as a result of buoyancy, mucoadhesivity, occlusion of the gastrointestinal tract, or arrest of peristalsis.

As disclosed herein, provided satiety modulating composition(s) are characterized as conferring stability towards one or more satiety modulator(s). Satiety modulating composition(s) known as prior art are characterized by poor resistance to aggregation, moisture uptake, and changes in porosity. In an unexpected result, one or more satiety modulator(s) present in one or more shell component(s) in provided satiety modulating composition(s) confer resistance to exchange of water and/or oils, thus preventing aggregation, moisture uptake, and porosity changes.

As disclosed herein, provided satiety modulating composition(s) are characterized as conferring stability towards one or more satiety modulator(s). Satiety modulating composition(s) known as prior art are characterized by changes to release profile(s) of one or more satiety modulator(s) upon a period of storage in dry air, humid air, aqueous conditions, oily conditions, and/or food and/or beverage matrices. In an unexpected result, one or more satiety modulator(s) present in one or more shell component(s) in provided satiety modulating composition(s) confer resistance to exchange of water and/or oils, thus preserving the release profile(s) expected in one or more biological environment(s) following a period of storage in dry air, humid air, aqueous conditions, oily conditions, and/or food and/or beverage matrices.

As disclosed herein, provided satiety modulating composition(s) are characterized as conferring stability towards one or more satiety modulator(s). Satiety modulating composition(s) known as prior art are characterized by susceptibility to chemical changes (e.g., hydrolysis, proteolysis, oxidation, reduction, lipolysis) that greatly reduce the efficacy in changing circulating satiety hormone concentration(s). Satiety modulating composition(s) known as prior art are characterized by susceptibility to chemical changes under a period of storage in dry air, humid air, aqueous conditions, oily conditions, and/or food and/or beverage matrices. Satiety modulating composition(s) known as prior art are characterized by susceptibility to chemical changes upon administration to one or more mammal(s). In an unexpected result, provided core-shell satiety modulating composition(s) confer resistance to chemical change under a period of storage in dry air, humid air, aqueous conditions, oily conditions, and/or food and/or beverage matrices by resisting water uptake and protecting satiety modulator(s) from light and oxygen. In an unexpected result, the inclusion of specific satiety modulator(s) within provided satiety modulating composition(s) enhances the chemical stability of satiety modulator(s) comprising provided satiety modulating composition(s). Without wishing to be bound by any particular theory, it is contemplated that the inclusion of sterols confers lipolytic resistance towards satiety modulating composition(s). Without wishing to be bound by any particular theory, it is contemplated that the inclusion of acaloric phytonutrients confers oxidative resistance towards satiety modulating composition(s).

As disclosed herein, provided satiety modulating composition(s) are characterized as solid(s). Satiety modulating composition(s) known as prior art are characterized as liquid(s), suffering from poor physical and chemical resistance and incompatibility with food and/or beverage matrices. In an unexpected result, the inclusion of specific satiety modulator(s) within provided satiety modulating composition(s) enhances the solidification of satiety modulator(s) comprising provided satiety modulating composition(s). Without wishing to be bound by any particular theory, it is contemplated that the inclusion of sterols confers solidification towards satiety modulating composition(s) comprising alkene-containing fatty acids.

As disclosed herein, provided satiety modulating composition(s) are characterized as providing satiety modulator(s) to the gastrointestinal tract of one or more mammal(s). Satiety modulating composition(s) known as prior art providing satiety modulator(s) to the gastrointestinal tract of one or more mammal(s) suffer from poor bioavailability of one or more satiety modulator(s). In an unexpected result, the inclusion of specific satiety modulator(s) within provided satiety modulating composition(s) enhances the absorption of one or more satiety modulator(s). Without wishing to be bound by any particular theory, it is contemplated that changes in circulating quantities of one or more satiety hormone(s) are sensitive to exposure to satiety modulator(s) in both the lumen and interstitial fluid, requiring satiety modulator absorption.

As disclosed herein, provided satiety modulating composition(s) are characterized as releasing 90% of provided satiety modulator(s) in the upper gastrointestinal tract and lower gastrointestinal tract of one or more mammal(s). Satiety modulating composition(s) known as prior art essentially release satiety modulator(s) in the lower gastrointestinal tract of one or more mammal(s), thus suffering from poor efficacy. In an unexpected result, provided core-shell preparation(s) comprise satiety modulator(s) in core component(s) and shell component(s), thus enabling targeting of both upper and lower gastrointestinal tract in one or more mammal(s). Without wishing to be bound by any particular theory, it is contemplated that changes in circulating quantities of one or more satiety hormone(s) are sensitive to exposure to satiety modulator(s) in both the lumen and interstitial fluid, requiring absorption (e.g., release in stomach) and malabsorption (e.g., release in ileum).

As disclosed herein, provided satiety modulating composition(s) are characterized by low water activity (e.g., less than 0.4). Satiety modulating composition(s) known as prior art exhibit high water activity (e.g., greater than 0.4), thus suffering from poor stability upon a period of storage in dry air, humid air, aqueous conditions, oily conditions, and/or food and/or beverage matrices. In an unexpected result, one or more satiety modulator(s) present in one or more shell component(s) in provided satiety modulating composition(s) confer resistance to exchange of water and/or oils, thus conferring low water activity to one or more satiety modulating composition(s).

As disclosed herein, provided methods of controlling satiety in one or more mammal(s) comprises a step of incorporating satiety modulating composition(s) into a food and/or beverage matrix. Methods of incorporating satiety modulating composition(s) known as prior art exhibit undesirable stability and homogeneity. In an unexpected result, incorporating satiety modulating composition(s) as provided herein exhibit desirable stability and homogeneity. Without wishing to be bound by any particular theory, it is contemplated that provided satiety modulating composition(s) are characterized by resistance to physical and/or chemical degradation, thus improving stability and homogeneity when dispersed in one or more food(s) and/or beverage(s).

As disclosed herein, provided methods of controlling satiety in one or more mammal(s) comprises a step of administering one or more satiety modulating composition(s) to one or more mammal(s). Methods of administering one or more satiety modulating composition(s) to one or more mammal(s) known as prior art suffer from poor efficacy by targeting a single gastrointestinal region. In an unexpected result, provided methods of administering satiety modulating composition(s) offer greatly enhanced efficacy by targeting multiple gastrointestinal regions.

As disclosed herein, provided methods of controlling satiety in one or more mammal(s) comprises a step of administering one or more satiety modulating composition(s) to one or more mammal(s). Methods of administering one or more satiety modulating composition(s) to one or more mammal(s) known as prior art do not substantially alter circulating satiety hormone concentration(s). In an unexpected result, provided methods of administering satiety modulating composition(s) offer greatly enhanced efficacy as characterized as change(s) in circulating satiety hormone concentration(s) of at least 20% relative to the post-absorptive state.

As disclosed herein, provided methods of controlling satiety in one or more mammal(s) comprises a step of administering one or more satiety modulating composition(s) to one or more mammal(s). Methods of administering one or more satiety modulating composition(s) to one or more mammal(s) known as prior art are effective for only short periods of time (e.g., less than 2 hours). In an unexpected result, provided methods of administering satiety modulating composition(s) offer greatly enhanced duration of effect as characterized as change(s) in circulating satiety hormone concentration(s) of at least 20% relative to the post-absorptive state. In some embodiments, provided methods of administering one or more satiety modulating composition(s) to one or more mammal(s) are characterized as effective for at least 6, at least 12, and/or at least 24 hours.

Additionally, or alternatively, satiety composition(s) may be used to control the (e.g., prolonging or accelerating) the interactions of satiety modulators with regions (e.g., ileum) or cells (e.g., L cells) in the gastrointestinal tract, provide luminal and basolateral integration of satiety modulator sensing, provide integration of satiety response elicited by several different satiety modulator(s), control the surface area or volume that satiety modulator(s) have access to in the gastrointestinal tract, control the rate at which the satiety modulators (e.g., lipids, proteins, carbohydrates, acaloric phytonutrients) release from the satiety modulating composition(s), decrease the absorption or bioavailability of nutrients, control satiety modulator spatial interactions within the host (e.g., controlled concentration of nutrients in the ileum and/or L cells and/or at the epithelial surface), and/or concentration of satiety modulator(s) in the gastrointestinal tract, or control interactions between satiety modulator(s) and gastrointestinal environments. Accordingly, these approaches may be beneficial to organisms (e.g., animals, e.g., humans) due to promoting and/or controlling interactions (e.g., concentration, duration, rate) in the gastrointestinal tract and/or in the ileum and/or on the L cells that govern satiety.

In certain embodiments, the present disclosure provides methods of prolonging postprandial duration of one or more mammal(s), the method comprising: providing an effective amount of satiety modulating composition(s) as described herein. In many embodiments, provided satiety modulating composition(s) are edible (i.e., consumable by eating). In some aspects, a method of orally administering satiety modulating composition(s) may be as a powder or slurry that is mixed with food (e.g., a freshly prepared meal, a pre-prepared meal, etc.) prior to consumption. In some aspects, a method of orally administering satiety modulating composition(s) may be as a drinkable composition as a powder or slurry that is mixed with a beverage (e.g., water, a protein shake, etc.) prior to consumption.

In some cases, a mammal may be a human. In some cases, a mammal may be a domesticated pet. In some cases, a mammal may be agricultural livestock. In some cases, humans may be an infant, toddler, child, teenager, adolescent, young adult, adult, geriatric, medical patient, athlete, student, etc.

In some embodiments, provided satiety modulating composition(s) may be or comprise one or more particles; typically, a population of particles (e.g., a particle preparation). In some embodiments, a particle or population thereof is characterized by its diameter (e.g., average diameter). A particle “diameter” (i.e., a particle size) is the longest distance from one end of the particle to another end of the particle. In some embodiments, food and/or beverage compositions (e.g., formulated ingestibles) are or comprise particles with a distribution of particle diameters (e.g., D[3,2], D[4,3], etc.). In some embodiments, satiety compositions (e.g., macronutrients, formulated protein, formulated fat, formulated carbohydrate) may include particle preparations that include particles with one or more of a variety of shapes or forms, for example, having a cross-section shape of a circle, an oval, a triangle, a square, a hexagon, or an irregular shape.

In some embodiments, the present disclosure provides satiety compositions (e.g., macronutrients, formulated protein, formulated fat, formulated carbohydrate) with digestion and/or degradation resistance (e.g., mitigation of satiety modulator digestions and/or degradation) to stomach acids, digestive enzymes (e.g., trypsinogen, chymotrypsinogen, elastase, carboxypeptidase, pancreatic lipase, nucleases and amylase), bile, pancreatic juices, peristaltic forces, and/or combinations thereof. The present disclosed satiety compositions (e.g., macronutrients, formulated protein, formulated fat, formulated carbohydrate), therefore, may provide benefits over existing products, among other things because digestion and/or degradation of satiety modulators may impair binding between satiety modulators and/or satiety receptors after ingestion, and/or upon contact with the stomach and/or stomach fluids and/or digestive fluids. Thus, the present disclosure provides technologies with a variety of advantages.

The present disclosure provides an insight that one challenge in using satiety compositions (e.g., macronutrients, formulated protein, formulated fat, formulated carbohydrate) may be identifying nutrients (e.g., macronutrients) and specific ratios of nutrients (e.g., ratios of proteins, fats, carbohydrates) that confer satiety modulation.

Without wishing to be bound by theory, many presently available food and/or beverage products cannot achieve control over satiety and therefore lack various benefits provided by the present disclosure. Technologies provided herein enable delivery and delivery functions to control satiety when individually ingested, and/or when combined with or into a multitude of food and/or beverages and/or powder products in the areas of supplements, foods, and/or beverages.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### V. BRIEF DESCRIPTION OF THE DRAWINGS

FIG. 1 shows, in a non-limiting example, a schematic of exemplary core-shell preparations with multiple layers which may comprise satiety modulators, core components, shell components, and combinations thereof. Additionally, or alternatively, exemplary multi-layer core-shell preparations may comprise a particle comprising at least one core-shell preparation, at least one core component, at least one shell component, at least one satiety modulator, or a combination thereof.

FIG. 2 shows, in a non-limiting example, a comparison of unformulated carbohydrate powder and satiety modulating composition(s) comprising carbohydrate(s) as component(s) of core-shell preparation(s). (A) Unformulated glucose; (B) sucrose-amylose core component(s) encapsulated in Zein; (C) 60% (w/v) glucose encapsulated in 2% (w/v) pectin; (D) 1% (w/v) inulin encapsulated in 2% (w/v) agarose; (E) 10% (w/v) calcium caseinate and 1% (w/v) inulin encapsulated in 2% (w/v) sodium alginate.

FIG. 3 shows, in a non-limiting example, a comparison of unformulated protein and satiety modulating composition(s) comprising protein(s) as component(s) of core-shell preparation(s). (A) Unformulated whey protein isolate (WPI) powder; (B) 20% (w/v) whey protein isolate encapsulated in 80% (w/v) beeswax, (C) whey protein (5% w/v) encapsulated in 80% (w/v) fully hydrogenated soy oil and Tween 80 (15% w/v); (D) whey protein (10% w/v) encapsulated in 3% (w/v) agarose and 1% (w/v) chitosan.

FIG. 4 shows, in a non-limiting example, a comparison of unformulated lipid and satiety modulating composition(s) comprising lipid(s) as component(s) of core-shell preparation(s). (A) Unformulated oleic acid (OLEA); (B) 80% (w/v) oleic acid in ethyl cellulose (20% w/v); (C) 80% (w/v) oleic acid in carnauba wax (20% w/v); (D) 80% (w/v) oleic acid in ethyl cellulose (20% w/v); (E) 80% (w/v) oleic acid in ethyl cellulose (20% w/v) with a polymeric coating.

FIG. 5 illustrates, in a non-limiting example, several exemplary release profiles of carbohydrate(s) encapsulated within one or more satiety modulating composition(s) in phosphate buffered saline, pH 7.4, 37° C. Satiety modulating composition(s) characterized as core component(s) are colored light grey, while those characterized as core-shell preparations are colored dark grey. (A) Release of glucose from satiety modulating composition(s) over time; each line represents an average of 3 dissolution experiments for a distinct satiety modulating composition (e.g., distinct component(s) and concentration(s)) comprising glucose. (B) First order release rates modeled from glucose release of distinct satiety modulating composition(s) sorted from fastest release (top) to slowest release (bottom).

FIG. 6 illustrates, in a non-limiting example, several exemplary release profiles of protein(s) encapsulated within one or more satiety modulating composition(s) in phosphate buffered saline, pH 7.4, 37° C. Satiety modulating composition(s) characterized as core component(s) are colored light grey, while those characterized as core-shell preparations are colored dark grey. (A) Release of whey from satiety modulating composition(s) over time; each line represents an average of 3 dissolution experiments for a distinct satiety modulating composition (e.g., distinct component(s) and concentration(s)) comprising whey. (B) First order release rates modeled from whey release of distinct satiety modulating composition(s) sorted from fastest release (top) to slowest release (bottom).

FIG. 7 illustrates, in a non-limiting example, several exemplary release profiles of lipid(s) encapsulated within one or more satiety modulating composition(s) in phosphate buffered saline, pH 7.4, 37° C. Satiety modulating composition(s) characterized as core component(s) are colored light grey. (A) Release of oleic acid from satiety modulating composition(s) over time; each line represents an average of 3 dissolution experiments for a distinct satiety modulating composition (e.g., distinct component(s) and concentration(s)) comprising oleic acid. (B) First order release rates modeled from oleic acid release of distinct satiety modulating composition(s) sorted from fastest release (top) to slowest release (bottom).

FIG. 8 illustrates, in a non-limiting example, a comparison of unformulated flavonoid (e.g., polyphenol) and formulated flavonoid (e.g., polyphenol) in satiety modulating composition(s) as components of core component(s) and/or core-shell preparation(s), as well as release profile(s) of encapsulated flavonoid(s). (A) Unformulated cyanidin chloride powder. (B) Core component comprising glucose. (C) core-shell preparation wherein core component(s) comprise glucose and shell component(s) comprise cyanidin chloride. (D) Release of cyanidin chloride from core-shell preparation in phosphate buffered saline, pH 7.4, 37° C.

FIG. 9 illustrates, in a non-limiting example, a comparison of unformulated carbohydrate and formulated carbohydrate in satiety modulating composition(s) as components of core component(s) and/or core-shell preparation(s), as well as release profile(s) of encapsulated carbohydrate(s). (A) Unformulated inulin powder (B) Core component comprising inulin. (C) Release of inulin from matrix preparation in phosphate buffered saline, pH 7.4, 37° C.

FIG. 10 shows, in a non-limiting example, cross-sectional micrographs of satiety modulating composition(s). (A) Cross-section of zein-coated sucrose particle preparation(s); (B) Cross-section of glucose 60% (w/v) encapsulated in pectin 1.5% (w/v); (C) Surface of glucose (10% w/v) encapsulated in 3% (w/v) agarose and 1% (w/v) inulin; (D) Surface of 20% (w/v) calcium caseinate encapsulated in 1% (w/v) inulin and 2.5% (w/v) alginate; (E) Cross-section of whey (5% w/v) in 80% (w/v) soy wax and 15% (w/v) tween-80 further encapsulated in a shell component comprising cellulose acetate phthalate; (F) Cross-section of whey (20% w/v) in 80% (w/v) beeswax; (G) 15% (w/v) glucose and 10% (w/v) whey protein isolate encapsulated in 3% (w/v) agarose, 0.1% (w/v) kappa carrageenan, and 0.1% (w/v) locust bean gum, encapsulated in a shell component comprising cellulose acetate phthalate; (H) Cross-section of 80% (w/v) oleic acid encapsulated in ethyl cellulose (20% w/v); (I) Cross-section of 80% (w/v) oleic acid encapsulated in candelilla wax (20% w/v); (J) Cross-section of 80% (W/v) oleic acid in ethyl cellulose (20% w/v) encapsulated in a shell component comprising cellulose acetate phthalate.

FIG. 11 shows, in a non-limiting example, a schematic of a method used to create a core-shell food and/or beverage composition, referred to herein as “core-shell”.

FIG. 12 shows, in a non-limiting example, that core-shell preparation(s) control the release of one or more food component(s). (A) Micrograph depicting uncoated sucrose-containing matrix preparation(s); (B) Zein-coated sucrose-containing matrix preparations. (C) Release of glucose from coated particle preparation(s) (white circles) is slower than uncoated particle preparations (black circles); (D) Unformulated whey protein isolate; (E) Whey protein isolate encapsulated in a core-shell preparation comprising a shell component of chitosan polyphosphate; (F) Chitosan-coated satiety modulating composition(s) release faster in simulated intestinal fluid (squares) vs. simulated gastric fluid (circles). (G) Release of whey protein from several distinct core-shell preparation(s); each line corresponds to the average (n=3) release of whey protein over time for a distinct core-shell preparation wherein the core component further comprises a matrix preparation (light grey curves) compared to average (n=3) release of whey protein over time for a similar matrix preparation without shell component (dark grey curve). (H) Photograph of core-shell preparation wherein the core component further comprises a matrix preparation comprising agarose and whey protein and the shell component further comprises a matrix preparation comprising zein and cyanidin chloride.

FIG. 13 shows, in a non-limiting example, that, in some instances, core-shell preparation(s) comprise encapsulated core component(s). (A) 10% (w/v) whey protein isolate, 65% (w/v) oleic acid encapsulated in 10% (w/v) carnauba wax and 15% (w/v) ethyl cellulose, further encapsulated in a shell component comprising cellulose acetate phthalate; (B) 60% (w/v) glucose encapsulated in 2% (w/v) pectin, further encapsulated in a shell component comprising cellulose acetate phthalate.

FIG. 14 shows, in a non-limiting example, a schematic of a method used to characterize dissolution and/or release of food and/or beverage composition, referred to herein as “dissolution” and/or “release”.

FIG. 15 shows, in a non-limiting example, exemplary release environment(s). (A) 92% sucrose/8% amylose (circle) vs. 92% sucrose/8% amylose encapsulated in 10% w/v zein (square) satiety modulating composition(s) exhibiting release in 10 mM phosphate buffered saline pH 7.4 with 1% (w/v) hydroxypropyl methylcellulose; (B) Whey protein isolate (10% w/v) encapsulated in 75% (w/v) cetyl ester wax and 15% (w/v) span 80 exhibiting release in 10 mM phosphate buffered saline pH 7.4; (C) 10% (w/v) whey protein isolate encapsulated in 75% (w/v) CITREM and 15% (w/v) carnauba wax exhibiting release in (hollow triangle) simulated intestinal fluid vs simulated gastric fluid (filled triangle).

FIG. 16 shows, in a non-limiting example, satiety modulating composition(s) comprising pH-responsive shell component(s). (A) Release of 5% (w/v) whey protein isolate encapsulated in 80% (w/v) fully hydrogenated soybean oil and 15% (w/v) Kolliphor P188 with cellulose acetate phthalate coating in simulated intestinal fluid (hollow circle) vs Eudragit EPO coating in simulated intestinal fluid (filled square); (B) Release of 15% (w/v) whey protein isolate encapsulated in 3% (w/v) agarose, 1% (w/v) kappa carrageenan, and 1% (w/v) locust bean gum with cellulose acetate phthalate coating in simulated gastric fluid (filled diamond) vs. with Eudragit E PO coating in simulated intestinal fluid (filled diamond).

FIG. 17 presents, in a non-limiting example, 4 theoretical release profiles (concentration of food component vs incubation period) of one or more satiety modulator(s) from one or more satiety modulating composition(s).

FIG. 18 presents, in a non-limiting example, that choice of satiety modulator(s) influences release of one or more satiety modulator(s). Release of whey protein isolate from 5% (w/v) agarose matrix preparation(s) comprising sodium carboxymethylcellulose (2% w/v) (white squares), tween-60 (1% w/v) (grey), and poly(acrylic acid) (2% w/v) (black squares).

FIG. 19 presents, in a non-limiting example, that concentration of satiety modulator(s) influences release of one or more satiety modulator(s). Release of whey protein isolate from formulations comprising varying amounts of candelilla wax and gelucire 50/13 (w/v): 75%/15%, (white circle), 85%/5% (gray circle), and 80%/15% (black circle).

FIG. 20 presents, in a non-limiting example, that core component(s) influence release of one or more satiety modulator(s). Release of whey protein isolate from formulations of different core component(s): 5% (w/v) agarose and 1% (w/v) hydroxypropylmethyl cellulose (white triangle); 75% (w/v) cetyl ester wax and 15% (w/v) Span 80 (gray triangle); 60% (w/v) 27-stearine, 15% (w/v) β-sitosterol w/v, and 10% (w/v) γ-oryzanol (black triangle).

FIGS. 21A-B show, in a non-limiting example that satiety modulating compositions demonstrate low (<0.20) water activity and low moisture content at 25° C.

FIGS. 22A-D illustrate, in a non-limiting example, brightfield micrographs of satiety modulating compositions (e.g., alginate/whey beads, gelatin/whey beads, and/or sucrose/amylose beads) blended homogeneously with commercially available food product (e.g., MIRE, Ensure, water), imparting minimal change and/or discernable change to visible appearance (e.g., color and texture).

FIG. 23 shows, in a non-limiting example, exemplary multi-layer core-shell particle preparation(s) controlling the release of protein satiety modulator(s). FIG. 23A illustrates a photograph of multi-layer core-shell particle preparation(s) comprising 50% (w/w) casein, 27% (w/w) starch, 9% (w/w) lactose, 4% (w/w) inulin, 5% (w/w) hypromellose, and 5% (w/w) ethyl cellulose produced through a wet granulation, extrusion, and spheronization process, followed by fluid bed coating to a total coating weight gain of 10% (w/w). Particles exhibit a 14-mesh size. FIG. 23B illustrates exemplary release of casein from exemplary multi-layer core-shell particle preparation(s) over 4 hours in 10 mM phosphate buffered saline, pH 7.4 with no shell (black circles), inner shell of Hypromellose and outer shell of ethyl cellulose (dark grey diamonds), or inner shell of ethyl cellulose and outer shell of hypromellose (light grey squares). Data are an average percent release with respect to loaded protein concentration (e.g., initial loading within exemplary multi-layer core-shell particles) of 3 replicates−/−standard deviation.

FIG. 24 shows, in a non-limiting example, exemplary pH-responsive matrix particle preparation(s) controlling the release of protein satiety modulator(s). FIG. 24A illustrates a micrograph of pH-responsive particle preparation(s) comprising 61% (w/w) sodium alginate and 29% (w/w) calcium caseinate produced through a spray drying process utilizing an ultrasonic nozzle. Particles size analysis yields an average particle diameter (e.g., Dv₅₀) of 19.2 μm. FIG. 24B illustrates exemplary release of casein from exemplary pH-responsive matrix particle preparation(s) over 4 hours in either simulated intestinal fluid, pH 6.8 (black circles) or simulated gastric fluid, pH 1 (grey squares). Data are an average percent release with respect to loaded protein concentration (e.g., initial loading within exemplary matrix particles) of 3 replicates+/−standard deviation.

FIG. 25 shows, in a non-limiting example, exemplary bile salt-resistive matrix particle preparation(s) controlling the release of fatty acid satiety modulator(s). FIG. 25A illustrates a micrograph of bile salt-resistive particle preparation(s) comprising 30% (w/w) 27-Stearine, 30% (w/w) CITREM, and 40% (w/w) linoleic acid produced through a hot melt homogenization process. Particle size analysis yields an average particle diameter (e.g., Dv₅₀) of 149 μm. FIG. 25B illustrates exemplary release of unformulated linoleic acid (black circles) or formulated linoleic acid (e.g., 30% (w/w) 27-Stearine, 30% (w/w) CITREM, 40% (w/w) linoleic acid) (grey squares) from exemplary bile salt-resistive matrix particle preparation(s) over 4 hours in simulated intestinal fluid, pH 6.8 with 0.2% (w/v) sodium taurocholate. Data are an average percent release with respect to loaded protein concentration (e.g., initial loading within exemplary matrix particles) of 3 replicates+/−standard deviation.

FIG. 26 shows, in a non-limiting example, exemplary pH-responsive core-shell particle preparation(s) controlling the release of carbohydrate satiety modulator(s). FIG. 26A illustrates a micrograph of pH-responsive core-shell particle preparation(s) comprising 50% (w/w) casein, 27% (w/w) starch, 9% (w/w) lactose, 4% (w/w) glucose, 5% (w/w) hypromellose acetate succinate, and 5% (w/w) sodium alginate produced through a wet granulation, extrusion, and spheronization process, followed by fluid bed coating to a total coating weight gain of 10% (w/w). Particles exhibit a 14-mesh size. FIG. 26B illustrates exemplary release of glucose from exemplary pH-responsive core-shell particle preparation(s) over 3 hours in either simulated intestinal fluid, pH 6.8 (black circles) or simulated gastric fluid, pH 1 (grey squares). Data are an average percent release with respect to loaded protein concentration (e.g., initial loading within exemplary multi-layer core-shell particles) of 3 replicates−/−standard deviation.

FIG. 27 shows, in a non-limiting example, exemplary pH-responsive core-shell particle preparation(s) controlling the release of protein payload(s). Exemplary release of protein from exemplary pH-responsive core-shell particle preparation(s) over 4 hours in either simulated intestinal fluid, pH 6.8 (green dashed line) or simulated gastric fluid, pH 1 (grey solid line). Data are an average percent release with respect to loaded protein concentration (e.g., initial loading within exemplary multi-layer core-shell particles) of 3 replicates+/−standard deviation.

FIG. 28 shows, in a non-limiting example, controlling one or more properties of particle preparation(s) by selecting method(s) of manufacture and incorporation thereof into commercial food and/or beverage products. FIG. 28A Macroscopic matrix preparation comprising a protein payload dispersed within a lipid matrix. FIG. 28B Macroscopic granulated and spheronized particle preparation(s) comprising protein payload dispersed within a carbohydrate matrix. FIG. 28C Microscopy of coarsely milled protein-containing particle preparation(s) with associated particle size histogram (FIG. 28F) and median particle diameter Dv₅₀. FIG. 28D Microscopy of finely milled protein-containing particle preparation(s) with associated particle size histogram (FIG. 28G) and median particle diameter Dv₅₀. FIG. 28E Microscopy of spray dried protein-containing particle preparation(s) with associated particle size histogram (FIG. 28H) and median particle diameter Dv₅₀. FIG. 28I commercial food and/or beverage powder with incorporation of spheronized protein-containing particle preparation; FIG. 28J uniform integration of finely milled protein-containing particle preparation into commercial food and/or beverage powder. FIG. 28K poor incorporation of particle preparation into aqueous suspension; FIG. 28L additional matrix component(s) leading to better incorporation; FIG. 28M uniform incorporation of improved particle preparation(s) into enteral nutrition (Nutren®, Nestle).

FIG. 29A shows, in a non-limiting example, line graphs of particle size distributions of three exemplary particle formulations of the present disclosure: (i) 50% (w/w) eicosapentaenoic acid, 15% (w/w) carnauba wax, 15% (w/w) stearic acid, 15% (w/w) ethyl cellulose 100 cP, and 5% (w/w) α-tocopherol (Left Panel); (ii) 25% (w/w) eicosapentaenoic acid, 50% (w/w) EUDRAGUARD® Protect, 15% (w/w) calcium carbonate, and 10% (w/w) α-tocopherol (Middle Panel); and (iii) 40% (w/w) eicosapentaenoic acid, 37% (w/w) whey protein isolate, 10% (w/w) sesame oil, 5% (w/w) diindolylmethane, 5% (w/w) sitosterol, 3% (w/w) α-tocopherol Right Panel).

FIG. 29B shows, in a non-limiting example, microscopic images of three exemplary particle formulations of the present disclosure: (i) 50% (w/w) eicosapentaenoic acid, 15% (w/w) carnauba wax, 15% (w/w) stearic acid, 15% (w/w) ethyl cellulose 100 cP, and 5% (w/w) α-tocopherol (Left Panel); (ii) 25% (w/w) eicosapentaenoic acid, 50% (w/w) EUDRAGUARD Protect, 15% (w/w) calcium carbonate, and 10% (w/w) α-tocopherol (Middle Panel); and (iii) 40% (w/w) eicosapentaenoic acid, 37% (w/w) whey protein isolate, 10% (w/w) sesame oil, 5% (w/w) diindolylmethane, 5% (w/w) sitosterol, 3% (w/w) α-tocopherol Right Panel).

FIG. 29C shows, in a non-limiting example, photographic images of three exemplary particle formulations of the present disclosure: (i) 50% (w/w) eicosapentaenoic acid, 15% (w/w) carnauba wax, 15% (w/w) stearic acid, 15% (w/w) ethyl cellulose 100 cP, and 5% (w/w) α-tocopherol (Left Panel); (ii) 25% (w/w) eicosapentaenoic acid, 50% (w/w) EUDRAGUARD Protect, 15% (w/w) calcium carbonate, and 10% (w/w) α-tocopherol (Middle Panel); and (iii) 40% (w/w) eicosapentaenoic acid, 37% (w/w) whey protein isolate, 10% (w/w) sesame oil, 5% (w/w) diindolylmethane, 5% (w/w) sitosterol, 3% (w/w) α-tocopherol Right Panel).

FIG. 29D shows, in a non-limiting example, photographic images of three exemplary particle formulations of the present disclosure incorporated into Chobani vanilla yogurt at 10% (w/v): (i) 50% (w/w) eicosapentaenoic acid, 15% (w/w) carnauba wax, 15% (w/w) stearic acid, 15% (w/w) ethyl cellulose 100 cP, and 5% (w/w) α-tocopherol (Left Panel); (ii) 25% (w/w) eicosapentaenoic acid, 50% (w/w) EUDRAGUARD Protect, 15% (w/w) calcium carbonate, and 10% (w/w) α-tocopherol (Middle Panel); and (iii) 40% (w/w) eicosapentaenoic acid, 37% (w/w) whey protein isolate, 10% (w/w) sesame oil, 5% (w/w) diindolylmethane, 5% (w/w) sitosterol, 3% (w/w) α-tocopherol Right Panel).

FIG. 30 shows, in a non-limiting example, a line graph of the permeability of eicosapentaenoic acid (EPA) in an unformulated (closed circles; 0.1% w/v EPA in 99.9% w/v water) and exemplary formulation of the present disclosure (open circles; 0.1% (w/v) EPA, 0.9% (w/v) ethanol, 4% (w/v) triacetin, 5% (w/v) Tween 80, and 90% (w/v) water) across a Caco-2 cell monolayer in a trans-well human intestinal permeability model. Data are the percentage ratio of cumulative mass of EPA collected in basolateral chambers to the mass of initial EPA dose. Each datapoint is an average of 3 independent replicates.

FIG. 31 shows, in a non-limiting example, a list of exemplary satiety modulating compositions, their associated core components and/or shell components, their respective concentrations, and release rates, according to some embodiments of the present disclosure.

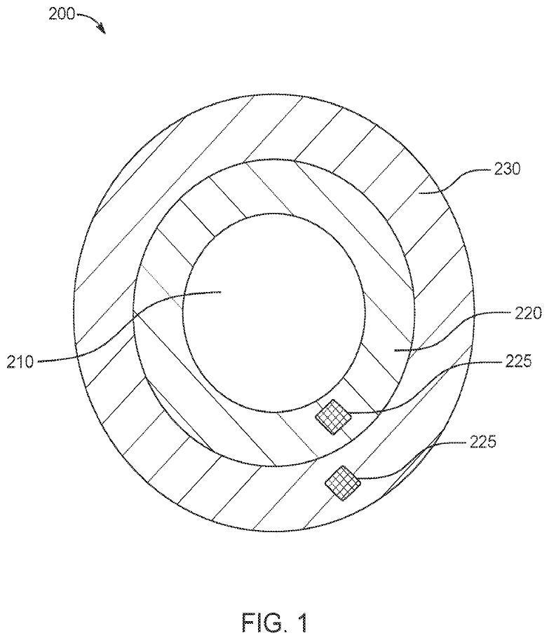

### VI. DETAILED DESCRIPTION OF CERTAIN EMBODIMENTS

Section headings used herein are for organizational purposes only and are not to be construed as limiting the subject matter described.

### A. Certain Terminology

Unless defined otherwise, technical and scientific terms used herein have the same meaning as is commonly understood to which the claimed subject matter belongs. In the event that there are a plurality of definitions for terms herein, those in this section prevail.

It is to be understood that the general description and the detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Definition of standard chemistry terms may be found in reference works, including but not limited to, Carey and Sundberg “Advanced Organic Chemistry 4th Ed.” Vols. A (2000) and B (2001), Plenum Press, New York.

As used herein, the symbol “<” means less than or fewer than. As used herein, the symbol “>” means more than.

As used herein, the term “about” or “approximately” means within 10%, preferably within 10%, and more preferably within 5% of a given value or range.

Ambient: The term “ambient”, as used herein, refers to a typical indoor (e.g., climate-controlled) temperature, usually within a range of about 18° C. to about 32° C., and/or typical indoor (e.g., climate-controlled) humidity, usually within a range of about 30% to 50%. In some embodiments, ambient temperature is within a range of about 20° C. to about 30° C.

Beverage: As used herein, the term “beverage” is used to refer to a potable liquid (e.g., that can be ingested, swallowed, drunk, or consumed by a person or animal without material risk to the person or animal). For example, beverage can be or comprise beer, juice, milk, a sports drink, tea, water, soda, yogurt, etc. In some embodiments, a “beverage” may be or comprise a pharmaceutical formulation in liquid form.

Biocompatible: As used herein, the term “biocompatible” is used to describe a characteristic of not causing significant detectable harm to living tissue when placed in contact therewith e.g., in vivo. In certain embodiments, materials are “biocompatible” if they are not significantly toxic to cells, e.g., when contacted therewith in a relevant amount and/or under relevant conditions such as over a relevant period of time. In certain embodiments, materials are “biocompatible” if their addition to cells in vitro results in less than or equal to 20% cell death, and/or their administration in vivo does not induce significant inflammation or other adverse effects.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Degradation: As used herein, the term “degradation” refers to a change in chemical structure and often involves breakage of at least one chemical bond. To say that a chemical compound is degraded typically means that the chemical structure of the chemical compound has changed (e.g., a chemical bond is broken). Common mechanisms of degradation include, for example, oxidation, hydrolysis, isomerization, fragmentation, or a combination thereof.

Delivery: As used herein, the term “delivery” is used to refer to the carrying and/or deposition and/or moving of nutrients (e.g., macronutrients, micronutrients, ketones, flavanols, prebiotics, etc.) and/or encapsulants to particular location (e.g., into and/or throughout the body). In some instances, for example, delivery may refer to payload delivery to the epithelial cells in the gastrointestinal tract. In some instances, for example, delivery may refer to payload delivery to into the blood stream (e.g., systemic absorption). In some instances, for example, delivery may refer to ingestion at the point of consumption for a shelf-stable satiety modulating composition containing a nutrient payload.

Diameter: As used herein, the term “diameter” is used to refer to the longest distance from one end of a particle to another end of the particle. Those skilled in the art will appreciate that a variety of techniques are available for use in characterizing particle diameters (i.e., particle sizes). In some instances, for example, size of particles (e.g., diameter of particles) can be measured by a Coulter Counter. In some instances, for example, size of particles (e.g., diameter of particles) can be measured by a Malvern Mastersizer. In some embodiments, a population of particles is characterized by an average size (e.g., D[3,2], D[4,3], etc.) and/or by particular characteristics of size distribution (e.g., absence of particles above or below particular sizes [e.g., Dv10, Dv20, Dv30, Dv40, Dv50, Dv60, Dv70, Dv80, Dv90, Dv99, etc.], a unimodal, bimodal, or multimodal distribution, etc.).

Dispersity: As used herein, the term “dispersity” is used to refer to the breadth of particle size distribution relative to the average particle size. In some instances, for example, size of particles (e.g., diameter of particles) can be measured by a Coulter Counter. In some instances, for example, size of particles (e.g., diameter of particles) can be measured by a Malvern Mastersizer. In some embodiments, the population of particles is characterized by, for example, an average size (e.g., Dv50) and, for example, a corresponding standard deviation. In some instances, the dispersity of a population of particles refers to double (e.g., 2-fold) the ratio of standard deviation (e.g., σ) to average particle diameter (e.g., Dv50).

Encapsulant: As used herein, the term “encapsulant” is used to refer to anything that is used to encapsulate a payload. For example, in many embodiments of the present disclosure, a payload component (e.g., a nutrient component) is described as being encapsulated by an encapsulant (e.g., polymer component, food component, material component, etc.).

Encapsulated: As used herein, the term “encapsulated” is used to refer to a characteristic of being physically associated with, and in some embodiments partly or wholly covered or coated. For example, in many embodiments of the present disclosure, a payload component (e.g., a microbe component and/or a nutrient component) is described as being encapsulated by a polymer component.

Food: As used herein, the term “food” is used to refer to an edible solid (e.g., that can be ingested, swallowed, chewed, or consumed by a person or animal without material risk to the person or animal). For example, food can be or comprise agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, tablet, yogurt, etc. In some embodiments, a “food” may be or comprise a pharmaceutical formulation in solid form. In some embodiments, a “food” may generally refer to a food and/or beverage product. In some embodiments, a “food” may generally refer to an edible object that is intended to confer a benefit (e.g., health, energy, nutrition, performance, well-being) on one or more animal(s).

Food Compositions: As used herein, the term “food compositions” is used to refer to an edible solid (e.g., that can be ingested, swallowed, chewed, or consumed by a person or animal without material risk to the person or animal) or an ingestible liquid (e.g., that can be ingested, swallowed, drank, or consumed by a person or animal without material risk to the person or animal) that provides health benefits resulting from controlled release, absorption, spatial access, concentration, and/or residence time of nutrients. For example, food compositions can be or comprise agricultural seed, dry powders, supplements, solid foods, beverages and/or drinks, etc. In some embodiments, a “food composition” may be or comprise a pharmaceutical formulation in solid form. In some embodiments, a “food composition” may be or comprise a pharmaceutical formulation in liquid form. In some embodiments, a “food composition” may generally refer to a food and/or beverage product. In some embodiments, a “food composition” may generally refer to an edible object that is intended to confer a benefit (e.g., health, energy, nutrition, performance, well-being) on one or more animal(s). Example food compositions (e.g., formulated ingestibles) include protein shakes, dry powders (e.g., baby formula, protein powder, drink mixes, coffee grinds), Meal Ready-to-Eat (MRE), Meal Ready-to-Drink (RTD), electrolyte beverages, sports beverages, hard seltzers (alcoholic seltzers), dry foods (e.g., rice, pasta), water, medical foods (e.g., Ready-to-drink low phenylalanine medical food), supplements, beer, wine, soda, coffee, fermented foods and beverages (e.g., yogurt, beer, etc.); for example, MREs, Gatorade, Truly, Ensure, PKU Sphere Liquid, etc.

Food and Beverage Compositions: As used herein, the term “food and beverage compositions” is used to refer to an edible solid (e.g., that can be ingested, swallowed, chewed, or consumed by a person or animal without material risk to the person or animal) or an ingestible liquid (e.g., that can be ingested, swallowed, drank, or consumed by a person or animal without material risk to the person or animal) that provides health benefits resulting from controlled release, absorption, spatial access, concentration, and/or residence time of nutrients. For example, food and beverage compositions can be or comprise agricultural seed, dry powders, supplements, solid foods, beverages and/or drinks, etc. In some embodiments, a “food and beverage composition” may be or comprise a pharmaceutical formulation in solid form. In some embodiments, a “food and beverage composition” may be or comprise a pharmaceutical formulation in liquid form. In some embodiments, a “food and beverage composition” may generally refer to a food and/or beverage product. In some embodiments, a “food and beverage composition” may generally refer to an edible object that is intended to confer a benefit (e.g., health, energy, nutrition, performance, well-being) on one or more animal(s). Example food and beverage compositions (e.g., formulated ingestibles) include protein shakes, dry powders (e.g., baby formula, protein powder, drink mixes, coffee grinds), Meal Ready-to-Eat (MRE), Meal Ready-to-Drink (RTD), electrolyte beverages, sports beverages, hard seltzers (alcoholic seltzers), dry foods (e.g., rice, pasta), water, medical foods (e.g., Ready-to-drink low phenylalanine medical food), supplements, beer, wine, soda, coffee, fermented foods and beverages (e.g., yogurt, beer, etc.); for example, MREs, Gatorade, Truly, Ensure, PKU Sphere Liquid, etc.

Formulated Beverages: As used herein, the term “formulated beverages” is used to refer to an ingestible liquid (e.g., that can be ingested, swallowed, drank, or consumed by a person or animal without material risk to the person or animal) that provides health benefits resulting from controlled release, absorption, spatial access, concentration, and/or residence time of nutrients. Examples include protein shakes, coffee, Meal Ready-to-Drink (RTD), electrolyte beverages, sports beverages, hard seltzers (alcoholic seltzers), water, medical foods (e.g., Ready-to-drink low phenylalanine medical food), supplements, beer, wine, soda, fermented foods and beverages (e.g., yogurt, beer, etc.).

Formulated Foods: As used herein, the term “formulated foods” is used to refer to an edible solid (e.g., that can be ingested, swallowed, chewed, or consumed by a person or animal without material risk to the person or animal) that provides health benefits resulting from controlled release, absorption, spatial access, concentration, and/or residence time of nutrients. Examples include dry powders (e.g., baby formula, protein powder, drink mixes), Meal Ready-to-Eat (MRE), yogurt, cheese, freshly prepared meals, frozen meals, etc.

Formulated Ingestibles: As used herein, the term “formulated ingestibles” is used to refer to an edible dosage form (e.g., that can be ingested, drank, swallowed, chewed, or consumed by a person or animal without material risk to the person or animal) such as a pill, capsule, tablet, etc. that provides health benefits resulting from controlled release, absorption, spatial access, concentration, and/or residence time of nutrients.

Formulated Meals: As used herein, the term “formulated meals” is used to refer to a solid meals (e.g., that can be ingested, swallowed, chewed, or consumed by a person or animal without material risk to the person or animal) such as microwavable meals, freshly prepared meals, frozen meals, MREs, etc., that provides health benefits resulting from controlled release, absorption, spatial access, concentration, and/or residence time of nutrients.

Formulated Supplements: As used herein, the term “formulated supplements” is used to refer to an edible dosage form (e.g., that can be ingested, swallowed, chewed, or consumed by a person or animal without material risk to the person or animal) such as a pill, capsule, tablet, etc. that provides health benefits resulting from controlled release, absorption, spatial access, concentration, and/or residence time of nutrients.

Hard Seltzers: As used herein, the term “hard seltzer” is used to refer to an ingestible liquid that contains alcohol and carbonated water.

HLB: As used herein, the term “HLB” is used to refer to the hydrophilic lipophilic balance that is an inherent property of, for example, a nonionic surfactant. In some instances, the HLB value of a given non-ionic surfactant is obtained from a commonly accessible tabular source. In some embodiments, non-ionic surfactants characterized as having a low HLB value (e.g., <8) are compatible emulsifiers for lipid systems. In some embodiments, nonionic surfactants characterized as having a high HLB value (e.g., >15) are compatible emulsifiers for aqueous systems. In some embodiments, non-ionic surfactants characterized as having an intermediate HLB value (e.g., >8 and <15) are compatible emulsifiers with both lipid and aqueous systems.

Homogenous: As used herein, the term “homogenous” means of substantially uniform structure and/or composition throughout.

Hydrophobic: As used herein, the term “hydrophobic” is used to refer to the propensity of a material to reject association, chemically and/or physically, with water. In some instances, a material characterized as being hydrophobic is biologically derived and/or synthetically derived. In some instances, a material characterized as being hydrophobic is a lipid, protein, and/or carbohydrate. In some instances, a material characterized as being hydrophobic is a polymer and/or small molecule. Alternatively, or additionally, in some embodiments, composites, mixtures, blends, or super-structures of several materials are collectively referred to as hydrophobic based on their observed propensity to reject association, chemically and/or physically, with water.

Incorporation: As used herein, the term “incorporation” is used to refer to a characteristic of being physically associated with, and in some embodiments, dispersed within, embedded within, or mixed in a bulk material (e.g., a lipid matrix component).

Layer: As used herein, the term “layer” typically refers to a material disposed above or below a distinguishable material. In some embodiments, a particular entity or preparation (e.g., particle preparation) is described as “layered” if it is prepared via a process in which a first material is laid down and then a second material is applied atop or underneath the first material (e.g., as by dipping or spraying, etc.); in some such embodiments, physical or chemical distinctness of layers may be maintained over time, whereas in some such embodiments, physical or chemical distinctness of layers may decay over time, at least at layer interface(s). Alternatively or additionally, in some embodiments, a particular sample or preparation may be described as layered, independent of its mode of preparation, so long as at a particular point in time and/or using a particular mode of assessment, distinct materials can be identified in a layered structure. In some embodiments, a “layered” particle may include one or more layers that wholly encapsulate a material below. In some embodiments, a “layered” particle may include one or more layers that does not wholly encapsulate a material below. In some embodiments, at least one layer of a layered preparation is or comprises a polymer, e.g., a hydrophobic polymer or hydrophilic polymer. In some embodiments, each layer of a layered preparation is or comprises a polymer, e.g., a pH responsive polymer or a temperature-responsive polymer.

Lipid: As used herein, the term “lipid” is used to refer to a class of chemical structures characterized as hydrophobic materials. In some instances, a lipid material is derived from a biological source. In other instances, a lipid material is derived from a synthetic source. In some instances, a lipid is comprised of one or more aliphatic alcohols and/or acids linked by glycerol and/or glycol moieties. In other instances, a lipid is comprised of aliphatic chains, linear conjugated, aromatic, and/or cyclic aliphatic moieties. In some embodiments, a lipid refers to a pure chemical entity. In other embodiments, a lipid refers to a mixture of several pure chemical entities. For example, lipids include, but are not limited to: paraffin wax, montan wax, microcrystalline wax, polyethylene wax, petrolatum wax, ozokerite wax, ceresin wax, beeswax, lanolin wax, spermaceti wax, tallow wax, lac wax, Chinese insect wax, ambergris wax, soy wax, carnauba wax, candelilla wax, coconut wax, palm kernel wax, rice bran wax, butyric acid, n-butanol, pentanoic acid, n-pentanol, hexanoic acid, n-hexanol, heptanoic acid, n-heptanol, caprylic acid, n-octanol, nonanoic acid, n-nonanol, capric acid, n-decanol, lauric acid, n-dodecanol, myristic acid, n-tetradecanol, palmitic acid, n-hexadecanol, stearic acid, n-octadecanol, arachidonic acid, n-icosanol, fatty alcohol monoglyceride ethers, fatty acid monoglyceride esters, fatty alcohol diglyceride ethers, fatty acid diglyceride esters, fatty alcohol triglyceride ethers, fatty acid triglyceride esters, fatty alcohol glycol monoether, fatty acid glycol monoesters, fatty alcohol glycol diethers, fatty acid glycol diesters, fatty alcohol poly(glycerol) ethers, fatty acid poly(glycerol) esters, fatty alcohol poly(glycol) ethers, fatty acid poly(glycol) esters, coconut oil, corn oil, cottonseed oil, olive oil, palm oil, peanut oil, rapeseed oil, safflower oil, sesame oil, soybean oil, sunflower oil, almond oil, pine nut oil, cashew oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, cholesterol, cholenic acid, ursolic acid, or betulinic acid.

Lyophilized: As used herein, the term “lyophilized” is used to refer to the end product of a process by which water is removed from a material via sublimation. In some instances, prior to sublimation of water, the material is cooled to <−10° C., <−20° C., <−30° C., and/or <−70° C. In some instances, prior to the sublimation of water, the pressure is lowered to <200 torr, <150 torr, <100 torr, <50 torr, <10 torr, <5 torr, and/or <1 torr. Those skilled in the art recognize that the cooling temperature and pressure influence the physicochemical properties of the end product; it is understood that “lyophilized” encompasses all suitable manners of cooling and vacuum protocol.

Medical Foods: As used herein, the term “medical foods” is used to refer to an edible dosage form (e.g., that can be ingested, swallowed, chewed, or consumed by a person or animal without material risk to the person or animal) such as a pill, capsule, tablet, etc. that provides health benefits resulting from controlled release, absorption, spatial access, concentration, and/or residence time of nutrients.

Nutraceutical: As used herein, the terms “nutraceutical” or “nutraceutical composition” refer to a substance or material that is or comprises a nutraceutical agent (e.g., a nutraceutical). Those skilled in the art will be aware of a variety of agents understood in the art to be nutraceutical agents such as, for example, agents that are or comprise one or more antioxidants, macronutrients, micronutrients, minerals, prebiotics, probiotics, probiotic powders, probiotic ingredients, probiotic food ingredients, probiotic supplement ingredients, prebiotics, vitamins, or combinations thereof. In some embodiments, a nutraceutical is or comprises a carotenoid compound such as α-lipoic acid, astaxanthin, adonixanthin, adonirubin, β-carotene, coenzyme Q10, lutein, lycopene, or zeaxanthin. In some embodiments, a nutraceutical is or comprises a vitamin such as vitamin D. In many embodiments, a nutraceutical agent is a natural product, and in certain such embodiments it is a product produced by plants. Many nutraceutical agents are compounds that have been reported or demonstrated to confer a benefit or provide protection against a disease in an animal or a plant. In some cases, nutraceuticals may be used to improve health, delay the aging process, protect against chronic diseases, increase life expectancy, or support the structure or function of the body of an animal, such as a human, a pet animal, an agricultural animal, or another domesticated animal. As such, as used in the present disclosure, the terms “nutraceutical composition,” “food preparation,” “food composition,” “particle preparation,” etc. may all be generally understood to describe compositions, preparations, and/or particles that include one or more food components (for example, encapsulated food component(s)).

Nutrient: As used herein, the term “nutrient” is used to refer to a nutraceutical, a macronutrient, a carbohydrate, a sugar, a polysaccharide, a dietary fiber, a fat, a fatty acid, a lipid, a short-chain fatty acid, a protein, an amino acid, a peptide, a micronutrient, a vitamin, a mineral, a carotenoid, an element, a ketone body, a prebiotic, a probiotic, a postbiotic, a bacteria, a yeast, a polyphenol, a flavonoid, an antioxidant, an electrolyte, a salt, a circadian rhythm modulator, a supplement, a nootropic, and/or a source of energy.

Particle: As used herein, the term “particle” is used to refer to a discrete physical entity, typically having a size (e.g., a longest cross-section, such as a diameter) within a range. For example, a particle can have a size of about 5-3000 μm, about 5-2000 μm, about 5-1000 μm, about 5-500 μm, about 5-50 μm, about 5-300 μm, about 5-200 μm, about 5-100 μm, about 5-50 μm, about 5-25 μm, or about 5-10 μm. In some embodiments, a particle may describe or include animal pellets ranging in size up to 1 mm, 5 mm, 10 mm, 25 mm, and even about 50 mm (about 2 inches) in diameter. A “particle” is not limited to a particular shape or form, for example, having a cross-section shape of a sphere, an oval, a triangle, a square, a hexagon, or an irregular shape. In some cases, particles can be solid particles. In some cases, particles can be liquid particles. In some cases, particles can be gel or gel-like particles. In some cases, particles may have a particle-in-particle structure wherein a layer of one material (e.g., one type of polymer component) encapsulates another material (e.g., another type of polymer component, which may itself encapsulate yet another, or rather may be or comprise a “core”—e.g., a polymer matrix core—of the particle).

Parts per million (ppm): As used herein, 1 ppm (“parts per million”) is equivalent to 1 milligram per liter (mg/L) or 1 milligram per kilogram (mg/kg).

Payload: In general, the term “payload”, as used herein, refers to an agent that may be delivered or transported by association with another entity. In some embodiments, such association may be or include a covalent linkage; in some embodiments such association may be or include non-covalent interaction(s). In some embodiments, association may be direct; in some embodiments, association may be indirect. The term “payload” is not limited to a particular chemical identity or type; for example, in some embodiments, a payload may be or comprise, for example, an entity of any chemical class including, for example, a nutrient, a lipid, a metal, a nucleic acid, a polypeptide, a saccharide (e.g., a polysaccharide), small molecule, or a combination or complex thereof. In some embodiments, a nutrient may include a lipid, a saccharide, a protein, etc. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, a payload may be or comprise an agent in isolated or pure form; in some embodiments, such agent may be in crude form.

pH Responsive: The term “pH-responsive” is used to refer to certain component(s) as described herein, and in particular means that the relevant component is characterized in that one or more aspects of its structure or arrangement is altered when exposed to a change in pH condition (e.g., to a particular pH and/or to a pH change of particular magnitude). In some embodiments, a polymer component is considered to be “pH-responsive” if, when the relevant polymer component is associated with a payload component in a particle preparation as described herein, the particle preparation releases the payload component under specific pH condition(s). In some embodiments, >90% of payload component is released from a particle preparation that includes a pH-responsive polymer component within 15 minutes when the particle preparation is exposed to a particular defined pH condition (e.g., within a range of defined pH values and/or at a specific pH value); in some embodiments, such release results when such contacting occurs at temperatures between 33-40° C., and in aqueous-based buffers of ionic strength ranging from 0.001-0.151 M (e.g., water, simulated gastric fluid, gastric fluid, simulated intestinal fluid, intestinal fluid) with osmolality between 1-615 mOsm/kg. In some embodiments, a pH-responsive component is one that degrades when exposed to a particular pH or pH change. Alternatively or additionally, in some embodiments, a pH-responsive component is one that becomes soluble, or significantly (e.g., by at least about 5%) increases its solubility when exposed to a particular pH level, or pH change. In some embodiments, a pH-responsive component includes one or more moieties whose protonation state changes at the relevant pH or in response to the relevant pH change. For example, in some embodiments, a pH responsive component includes one or more amine moieties that become protonated upon exposure to a relevant pH or pH change. For example, in some embodiments, a pH responsive component includes one or more carboxylate moieties that become deprotonated upon exposure to a relevant pH or pH change.

Polyphenols: As used herein, the term “polyphenol” is used to refer to naturally occurring organic compounds, comprising one or multiple aromatic groups with one or more hydroxyl groups or hydroxyl derivatives (e.g., methoxyl, ethoxyl, acetyl, etc.) and/or deriving from the shikimate, phenylpropanoid, and/or polyketide pathways. For example, a polyphenol may be phenolic acids, flavonoids, stilbenes, and lignans, antioxidants, tannins, and/or combinations thereof.

Prebiotic: As used herein, the term “prebiotic” is used to refer to a non-digestible food ingredient that promotes the growth of beneficial microorganisms in the intestines.

Reference: As used herein describes a standard or control relative to which a comparison is made. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Residual solvent: As used herein, the term “residual solvent” refers to a solvent that remains in a material after manufacture or processing of the material. In some embodiments, level of residual solvent is assessed by HPLC, mass spec, NVIR, FTIR, and/or gas chromatography.

Satiety: As used herein, the term “satiety” refers to being full and/or sated; for example, feeling satisfied due to ingestion of a food and/or beverage composition or having a desire removed following ingestion of a food and/or beverage composition.

Satiety response: As used herein, the term “satiety response” refers to a change in one or more satiety hormones (e.g., leptin, GLP-1, GLP-2, motilin, gastrin, insulin, ghrelin, peptide yy, cholecystokinin, GIP, serotonin, histamine, oleoylethanolamide, palmitoylethanolamide, anandamide, linolenoylethanolamide, their prehormones, their isoforms, their degradation products, and/or their full length and/or spliced transcripts) upon exposure of one or more mammal(s) to nutrients.

Stable: The term “stable,” when applied to compositions herein, means that the compositions maintain (e.g., as determined by one or more analytical assessments) one or more aspects of their physical structure and/or performance characteristic(s) (e.g., activity) over a period of time and/or under a designated set of conditions. When an assessed composition is a particle composition, in some embodiments, as will be clear from context to those skilled in the art, the term “stable” refers to maintenance of a characteristic such as average particle size, maximum and/or minimum particle size, range of particle sizes, and/or distribution of particle sizes (i.e., the percentage of particles above a designated size and/or outside a designated range of sizes) over a period of time and/or under a designated set of conditions. For food and/or beverage compositions, stable often refers to maintenance or preservation of delivery functions (e.g., controlled release, sustained release, controlled residence time, sustained residence time, etc.).

Temperature-responsive: As used herein, the term “temperature-responsive” is used to refer to certain component(s) as described herein, and in particular means that the relevant component is characterized in that one or more aspects of its structure or arrangement is altered when exposed to a change in temperature condition (e.g., to a particular temperature and/or to a temperature change of particular magnitude). In some embodiments, a component is considered to be “temperature-responsive” if, when the relevant component is associated with a payload component in a particle preparation as described herein, amorphous regions of the component experience a transition from a rigid state (e.g., glassy state) to a more fluid-like flexible state (e.g., more conducive to flow), at a temperature close to the point of transition from the solid state to rubbery state (e.g., glass transition).

Water activity: As used herein, “water activity” of a material is an indication (e.g., a measurement) of how much free (i.e., available to bind or react) water is present in the material, and is typically determined as the ratio of the vapor pressure of water in a material (p) to the vapor pressure of pure water (p0) at the same temperature. For example, a water activity of 0.80 means the vapor pressure is 80 percent of that of pure water. Water activity typically increases with temperature. Those skilled in the art will be familiar with three basic water activity measurement systems: Preventive Electrolytic Hygrometers (REH), Capacitance Hygrometers, and Dew Point Hygrometers (sometimes called chilled mirror).

### B. Overview

Disclosed herein, among other things, are satiety modulating compositions (e.g., particle preparations that enhance and/or decrease satiety in a mammal) comprising a lipid, a protein, a carbohydrate, and an acaloric phytonutrient, and methods of controlling the satiety of a mammal utilizing satiety modulating compositions.

In certain embodiments of the present disclosure, provided satiety modulating compositions are comprised of one or more satiety modulators. In certain embodiments, one or more satiety modulators includes a lipid. In certain embodiments, one or more satiety modulators includes a protein. In certain embodiments, one or more satiety modulators includes a carbohydrate. In certain embodiments, one or more satiety modulators includes an acaloric phytonutrient. In certain embodiments, one or more satiety modulators includes a combination of lipids, proteins, carbohydrates, and/or acaloric phytonutrients. Without wishing to be bound by any particular theory, it is contemplated that oral administration of one or more satiety modulators is alone insufficient to control the satiety of one or more mammals.

In certain embodiments of the present disclosure, provided satiety modulators are spatially arranged (e.g., formulated) within a satiety modulating composition (e.g., a particle preparation). In certain embodiments, a satiety modulating composition (e.g., a particle preparation) comprises a core completely encapsulated by one or more chemically and/or physically distinct shell(s) to yield, e.g., a core-shell particle. In certain embodiments, one or more satiety modulators are present in a core of a core-shell particle. In certain embodiments, one or more satiety modulators are present in a shell of a core-shell particle. In certain embodiments, one or more satiety modulators are present in both a core and a shell of a core-shell particle. In certain embodiments, a satiety modulating composition (e.g., a particle preparation) disclosed herein is characterized as a solid, a gel, a crystal, a blend, a matrix, an aerosol, or a liquid. Without wishing to be bound by any particular theory, it is contemplated that spatial arrangement of one or more satiety modulators within a satiety modulating composition (e.g., a particle preparation) provides for the controlled delivery of one or more satiety modulators.

In certain embodiments of the present disclosure, administration of a satiety modulator composition (e.g., a particle preparation) to one or more mammals is characterized by controlled delivery and/or release of one or more satiety modulator. In certain embodiments, controlled delivery and/or release of one or more satiety modulators is achieved via a chemical and/or a physical property of a satiety modulating composition (e.g., a particle preparation). In certain embodiments, controlled delivery and/or release of one or more satiety modulators is achieved via a chemical and/or a physical property of a satiety modulator. In certain embodiments, controlled delivery and/or release of one or more satiety modulators is achieved via a selection and/or an arrangement (e.g., formulation) of one or more satiety modulators within a satiety modulating composition. In certain embodiments, controlled delivery and/or release of one or more satiety modulators is characterized by protection from degradation (e.g., proteolysis, lipolysis, glycolysis, oxidation, reduction), spatial targeting (e.g., release in the oral cavity, esophagus, stomach, duodenum, jejunum, ileum, colon, and/or rectum), temporal targeting (e.g., release after at least about 1, 2, 4, 6, 8, 12, 16, and/or 24 hours), biological retention (e.g., buoyancy, occlusion, mucoadhesivity), and/or increased bioavailability. Without wishing to be bound by any particular theory, it is contemplated that optimized delivery of one or more satiety modulators enables a lower effective dose relative to unoptimized delivery of one or more satiety modulators.

In certain embodiments of the present disclosure, a method of prolonging postprandial duration (e.g., providing for satiety) includes providing one or more satiety modulating compositions (e.g., a particle preparation) in an effective dose. In certain embodiments, a satiety modulating composition (e.g., a particle preparation) comprises a mass of one or more satiety modulators. In certain embodiments, the total mass of one or more satiety modulators is sufficient to control the satiety of one or more mammals. In certain embodiments, the total mass of a combination of satiety modulators is sufficient to control the satiety of one or more mammals.

In certain embodiments, the present disclosure provides a satiety modulating composition (e.g., a particle preparation) comprised of at least one lipid, at least one protein, and at least one carbohydrate. Additionally, or alternatively, the present disclosure provides a satiety modulating composition (e.g., a particle preparation) including a combination of one or more lipids, one or more proteins, and one or more carbohydrates that are released in both the upper and lower gastrointestinal tract. Additionally, or alternatively, in some embodiments, a satiety modulating composition (e.g., a particle preparation) of the present disclosure is a solid at least at about 20° C., 25° C., 30° C., 35° C., and/or 37° C.

Disclosed herein, among other things, are methods of prolonging postprandial duration (e.g., controlling the satiety) of one or more mammals. Without wishing to be bound by any particular theory, it is contemplated that the provided methods of controlling the satiety of one or more mammals is optimally achieved using a satiety modulating composition (e.g., a particle preparation), as provided herein.

In certain embodiments, a satiety modulating composition (e.g., a particle preparation) of the present disclosure is essentially non-therapeutic. Without wishing to be bound by any particular theory, one or more satiety modulators are naturally occurring and/or derivatives of natural products. Without wishing to be bound by any particular theory, one or more satiety modulators are not approved for the medical treatment of one or more metabolic disorders in the United States.

In certain embodiments, a method of prolonging postprandial duration (e.g., controlling the satiety) of one or more mammals is comprised of a step of administering an effective quantity of a satiety modulating composition (e.g., a particle preparation). In certain embodiments, administration of an effective quantity of a satiety modulating composition (e.g., particle preparation) is via an oral route (e.g., drinking, eating, chewing, swallowing, etc.). In certain embodiments, administration of an effective quantity of one or more satiety modulators is achieved through the administration of one or more satiety modulating compositions (e.g., a particle preparation), as provided herein. Without wishing to be bound by any particular theory, it is contemplated that an effective quantity of a satiety modulator is a sufficient quantity to elicit a satiety response, yet small enough quantity to avoid caloric utilization and/or sensory impact with one or more co-administered foods and/or beverages. In certain embodiments, a satiety modulator composition (e.g., particle preparation) is administered as a solution, suspension, elixir, powder, tablet, capsule, patch, and/or pouch further comprising a food, beverage, nutraceutical, and/or pharmaceutical product.

In certain embodiments, a method of prolonging postprandial duration (e.g., controlling the satiety) of one or more mammals is achieved over a predetermined duration. In some embodiments, a method of controlling the satiety of one or more mammals is efficacious over at least 2, at least 4, at least 6, at least 8, at least 10, at least 12, at least 16, and/or at least 24 hours. In certain embodiments, the effective quantity of one or more satiety modulators determines the duration of satiety control. In certain embodiments, duration of satiety control may be determined by one or more of: the selection and/or spatial arrangement (e.g., formulation) of one or more satiety modulators within a satiety modulating composition (e.g., a particle preparation; one or more oleogelators present in a satiety modulating composition; and one or more pH-responsive carbohydrates in a satiety modulating composition. In certain embodiments, the duration of satiety control is determined by a satiety modulating composition (e.g., a particle preparation) characterized by single mode of controlled delivery. In certain embodiments, the duration of satiety control is determined by two or more satiety modulating compositions (e.g., particle preparations), each characterized as having a different mode of controlled delivery as the other.

As provided herein, a method of controlling the satiety of one or more mammals comprises one or more steps, each of which may occur sequentially. Additionally, or alternatively, a method of controlling the satiety of one or more mammals, as provided herein, comprises one or more steps, each of which may occur contemporaneously.

The present disclosure provides a method of controlling satiety characterized in that at least about 75%, at least about 80%, at least about 85%, at least about 90%, and/or at least about 95% of an administered dose of one or more satiety modulators is released in the ileum of one or more mammals. Additionally, or alternatively, the present disclosure provides a method of controlling satiety characterized in that at least about 75%, at least about 80%, at least about 85%, at least about 90%, and/or at least about 95% of an administered dose of one or more satiety modulators is released in the stomach and/or duodenum of one or more mammals. Additionally, or alternatively, the present disclosure provides a method of controlling satiety characterized in that at least about 90% of an administered dose of one or more satiety modulators is released in the gastrointestinal tract of one or more mammals.

In some embodiments, the present disclosure provides a satiety modulating composition (e.g., a particle preparation) characterized as avoiding deleterious side effects including hepatotoxicity, cardiotoxicity, and/or malabsorption of nutrients commonly found in satiety modulating composition(s) known to those skilled in the art.

### C. Satiety Modulating Compositions

In some embodiments of the present disclosure, various satiety modulating composition(s) comprising one or more lipid(s), one or more protein(s), one or more carbohydrate(s), and/or one or more acaloric phytonutrient(s) are provided. In certain embodiments, one or more lipid(s), one or more protein(s), one or more carbohydrate(s), and/or one or more acaloric phytonutrient(s) exhibit a predetermined physical arrangement (e.g., formulation). In certain embodiments, one or more lipid(s), one or more protein(s), one or more carbohydrate(s), and/or one or more acaloric phytonutrient(s) exhibit an non-uniform physical arrangement.

In certain embodiments, one or more satiety modulating composition(s) is comprised of a single lipid, a single protein, a single carbohydrate, and/or a single acaloric phytonutrient. In certain embodiments, one or more satiety modulating composition(s) is comprised of multiple lipids, multiple proteins, multiple carbohydrates, and/or multiple acaloric phytonutrients. In certain embodiments, one or more satiety modulating composition(s) is comprised of a single physical arrangement of satiety modulator(s). In certain embodiments, one or more satiety modulating composition(s) is comprised of multiple physical arrangements of satiety modulator(s).

In certain embodiments, a satiety modulating compositions (e.g., particle preparation) disclosed herein exhibits functional characteristics as a result of one or more of: the selection of one or more satiety modulators within a satiety modulating composition; the protection of one or more satiety modulators from degradation; the solidification of a satiety modulating composition a physico-chemical response to an environmental and/or chemical trigger; the biological retention of one or more satiety modulating compositions; and/or the bioavailability of one or more satiety modulators. In certain embodiments, one or more satiety modulator(s) exhibit a single functional characteristic, as provided herein. In certain embodiments, one or more satiety modulator(s) exhibit multiple functional characteristics, as provided herein. In certain preferred embodiments, functional characteristics exhibited by one or more satiety modulator(s) determines controlled release exhibited by one or more satiety modulating composition(s) (e.g., a particle preparation).

In certain embodiments, the release of one or more satiety modulator(s) from one or more satiety modulating composition(s) is or may be characterized as controlled release. For example, in certain embodiments, controlled release is or may be defined as at least one of release in response to time, release in response to a physical trigger, release in response to a chemical trigger, release in response to an environmental trigger, and/or release in a specific biological compartment. In certain embodiments, release of one or more satiety modulators, is characterized by the amount of one or more satiety modulator(s) transferred from one or more satiety modulating composition(s) to its surrounding environment (e.g., a release environment). In certain embodiments, a release profile is characterized as an amount of one or more satiety modulators released in response to time, in response to a physical trigger, in response to a chemical trigger, in response to an environmental trigger, and/or in response to a specific biological compartment.

In certain embodiments, one or more satiety modulating composition(s) is characterized as amenable to incorporation into food and/or beverage products. In certain preferred embodiments, satiety modulating compositions are further characterized as exhibiting suitable water activity, stability, and/or minimal sensory impact when incorporated into food and/or beverage products.

In certain embodiments, one or more satiety modulating composition(s) is characterized as providing one or more satiety modulator(s) in an effective quantity. In certain embodiments, one or more satiety modulating composition(s) provides an effective quantity of a single satiety modulator. In certain embodiments, one or more satiety modulating composition(s) provides an effective quantity of multiple satiety modulators. In certain embodiments, an effective dose is characterized by a mass of satiety modulator(s) provided. In certain embodiments, an effective dose is characterized by a caloric value of the satiety modulator(s) provided.

### 1. Satiety Modulating Compositions

In some embodiments, satiety modulating composition(s) are comprised of one or more satiety modulator(s). In certain embodiments, satiety modulating composition(s) are comprised of a predetermined quantity of one or more satiety modulator(s) expressed as a relative percent, on a dry weight basis, of the total composition.

### (i) Satiety Modulator(s)

In certain embodiments, one or more components comprising the provided satiety modulating composition(s) are characterized as satiety modulators. Typically, as described herein, one or more satiety modulators is or are characterized as being at least one of a lipid, a protein, a carbohydrate, and/or an acaloric phytonutrient. Additionally, or alternatively, one or more components characterized as a satiety modulator is a component known in the prior art to elicit a satiety response; for example, a feeling of fullness in a mammal, a feeling of satisfaction in a mammal, a craving in a mammal, an elevated systemic plasma concentration of leptin, an elevated systemic plasma concentration of GLP-1, an elevated systemic plasma concentration of GLP-2, an elevated systemic plasma concentration of motilin, an elevated systemic plasma concentration of gastrin, an elevated systemic plasma concentration of insulin, an elevated systemic plasma concentration of ghrelin, an elevated systemic plasma concentration of peptide yy, an elevated systemic plasma concentration cholecystokinin, an elevated systemic plasma concentration of GIP, an elevated gastrointestinal plasma concentration of serotonin, an elevated cerebrospinal fluid concentration of histamine, an elevated gastrointestinal plasma concentration of oleoylethanolamide, an elevated gastrointestinal plasma concentration of palmitoylethanolamide, and/or an elevated gastrointestinal plasma concentration of linolenoylethanolamide as compared to basal levels.

As provided herein, a satiety modulating composition (e.g. a particle preparation) comprising one or more satiety modulators is essentially non-therapeutic. In certain embodiments, one or more satiety modulator(s) is derived from natural sources and used either with or without modification. In some embodiments, one or more satiety modulator(s) is a pure chemical entity. In some embodiments, one or more satiety modulator(s) is a combination of several chemical entities. In certain embodiments, one or more satiety modulator(s) is not approved for treatment of one or more metabolic disorders by the United States Food and Drug Administration.

In certain embodiments, one or more satiety modulator(s) is characterized by receptor-mediated interactions with cells present in the gastrointestinal tract of one or more mammal(s). For example, one or more satiety modulator(s) is or may be characterized as interacting with receptors present on cells comprising the oro-buccal cavity, esophagus, stomach, duodenum, jejunum, ileum, cecum, colon, and/or rectum of one or more mammal(s). For example, one or more satiety modulator(s) is or may be characterized as interacting with at least one of enterocytes, dendritic cells, goblet cells, Paneth cells, neuroendocrine cells, afferent neurons, Tuft cells, M cells, K cells, L cells, smooth muscle cells, fibroblasts, adipocytes, mast cells, macrophages, and/or lymphocytes. For example, one or more satiety modulator(s) is or may be characterized as being a ligand of at least one isoform of: GPRC6A, CaSR, TasteR, GPR93, FFAR2, FFAR3, FFAR1, FFAR4, GPR40, GPR119, CB1, and/or GPR120.

As provided herein, in certain embodiments, a satiety modulating composition comprises at least about 75%, at least about 80%, at least about 85%, at least about 90%, and/or at least about 93%, on a dry weight basis, of one or more satiety modulators. In certain embodiments, a satiety modulating composition (e.g., a particle preparation) includes multiple satiety modulators, together comprising at least about 75%, at least about 80%, at least about 85%, at least about 90%, and/or at least about 930, on a dry weight basis, of the satiety modulating composition (e.g., particle preparation).

In certain embodiments, one or more satiety modulator(s) comprises a lipid. In some embodiments, a lipid comprises a fatty acid, a fatty amide, a sterol, a stanol, a terpene, or a terpenoid. In certain embodiments, a lipid comprises one or more alkane (e.g., sp³ carbons), alkene (e.g., sp² carbons), and/or alkyne (e.g., sp carbons) moieties. In certain embodiments, a lipid comprises one or more alkene moieties. In certain embodiments, one or more lipid(s) characterized as possessing one or more alkene moieties is further characterized as an unsaturated fatty acid or unsaturated fatty amide.

In some embodiments, a satiety modulating composition (e.g., a particle preparation) comprises one or more lipids. In some embodiments, a satiety modulating composition (e.g., a particle preparation) comprises about 40% to about 75% (e.g., about 40% to about 75%, about 45% to about 75%, about 50% to about 75%, about 55% to about 75%, about 60% to about 75%, about 65% to about 75%, about 70% to about 75%, about 40% to about 65%, about 45% to about 65%, about 50% to about 65%, about 55% to about 65%, about 60% to about 65%, about 40% to about 55%, about 45% to about 55%, or about 40% to about 45%), on a dry weight basis, of one or more lipids. In some embodiments, a satiety modulating composition (e.g., a particle preparation) comprises about 40% to about 75% (e.g., about 40% to about 75%, about 45% to about 75%, about 50% to about 75%, about 55% to about 75%, about 60% to about 75%, about 65% to about 75%, about 70% to about 75%, about 40% to about 65%, about 45% to about 65%, about 50% to about 65%, about 55% to about 65%, about 60% to about 65%, about 40% to about 55%, about 45% to about 55%, or about 40% to about 45%), on a dry weight basis, of one or more lipids in a core of the satiety modulating composition (e.g., a core-shell particle preparation). In some embodiments, a satiety modulating composition (e.g., a particle preparation) comprises one or more lipids in both a core and a shell of the satiety modulating composition (e.g., a core-shell particle preparation). In some embodiments, a satiety modulating composition (e.g., a particle preparation) comprises one or more lipids in a shell of the satiety modulating composition (e.g., a core-shell particle preparation).

In some embodiments, one or more lipids include an unsaturated fatty acid and/or a fatty amide. In some embodiments, an unsaturated fatty acid and/or a fatty amide may comprise at least 8, at least 10, at least 12, at least 14, at least 16, at least 18, at least 20, and/or at least 22 carbons. In some embodiments, one or more lipids include an unsaturated and/or an unconjugated fatty acyl. In some embodiments, an unsaturated and/or an unconjugated fatty acly comprises less than about 26 carbons. In some embodiments, one or more lipids include an unsaturated fatty acid and/or a fatty amide. In some embodiments an unsaturated fatty acid and/or a fatty amide comprises at least 1, at least 2, at least 3, at least 6, at least 9, and/or at least 12 double bonds. In some embodiments, an unsaturated fatty acid and/or a fatty amide may comprise two sp³ hybridized carbons adjacent to all sp² hybridized carbons. In some embodiments, an unsaturated fatty acid and/or a fatty amide is all-cis. In some embodiments, an unsaturated fatty acid and/or a fatty amide comprises a +3 oxidized carbon.

For example, in some embodiments, one or more satiety modulator(s) includes all-cis α-linolenic acid, γ-linolenic acid, anandamide, arachidonic acid, adrenic acid, calendic acid, clupanodonic acid, docosadienoic acid, docosapentaenoic acid, docosahexaenoic acid, eicosadienoic acid, eicosapentaenoic acid, eicosatrienoic acid, elaidic acid, erucic acid, gondoic acid, linoleamide, linoleic acid, linolenylamide, mead acid, N-docosahexaenoylethanolamine, N-docosatetraenoylethanolamine, N-oleoylethanolamine, N-palmitoylethanolamine, N-stearoylethanolamine, nervonic acid, nisinic acid, oleamide, oleic acid, osbond acid, palmitoleic acid, pinolenic acid, paullinic acid, rumenic acid, stearidonic acid, tetracosapentaenoic acid, vaccenic acid, or any combination thereof.

In some embodiments, one or more satiety modulator(s) may include an unmodified fatty acid and/or an unmodified fatty amide. In some embodiments, a fatty acid and/or a fatty amide may include an ester, an amide, and/or an ether to a glycerol backbone as a monoglyceride, a diglyceride, and/or a triglyceride. Alternatively, or additionally, in some embodiments, a fatty acid and/or a fatty amide may include an ester, an amide, and/or an ether to ethylene glycol, propylene glycol, poly(ethylene glycol), poly(propylene glycol), sucrose, maltose, xylitol, erythritol, sorbitol, and/or mannitol.

In some embodiments, one or more satiety modulator(s) includes a sterol, a stanol, a terpene, and/or a terpenoid. In some embodiments, a sterol, a stanol, a terpene, and/or a terpenoid is derived from plants without further modification. In some embodiments, one or more satiety modulator(s) includes γ-oryzanol, abietane, abietic acid, brassicasterol, campestanol, campesterol, cholestanol, cholesterol, ergosterol, sitostanol, sitosterol, oleanolic acid, ursolic acid, betulinic acid, moronic acid, cafestol, limonene, hinokitiol, carvone, menthol, linalool, thujene, stigmasterol, or any combination thereof. In some embodiments, one or more satiety modulator(s) is characterized as conferring resistance to water, resistance to oxygen, resistance to acid, resistance to ultraviolet light, solidification, or any combination thereof.

In certain embodiments, one or more satiety modulator(s) comprises one or more proteins. In some embodiments a protein may comprise a dipeptide, a tripeptide, an oligopeptide, and/or a polypeptide. In some embodiments, one or more satiety modulator(s) comprises one or more L-amino acids. In some embodiments, one or more proteins may be characterized as possessing a molecular weight of at least about 73 Da, about 200 Da, about 500 Da, about 1000 Da, about 5000 Da, about 20000 Da, about 100000 Da, and/or about 500000 Da. In some embodiments, one or more proteins may be characterized as being enzymatically active. In other embodiments, one or more proteins may be characterized as being enzymatically inactive. In some embodiments, one or more proteins is characterized as having endocrine activity. In some embodiments, one or more proteins is characterized as having nutritional content (e.g., caloric content). In some embodiments, one or more proteins is extracted from natural sources without substantial modification and/or purification. In some embodiments, one or more proteins is extracted from natural sources and substantially modified and/or purified.

In some embodiments, a satiety modulating composition (e.g., a particle preparation) comprises about 10% to about 30% (e.g., about 10% to about 30%, about 15% to about 30%, about 20% to about 30%, about 25% to about 30%, about 10% to about 25%, about 15% to about 25%, about 20% to about 25%, about 10% to about 20%, about 15% to about 20%, or about 10% to about 15%), on a dry weight basis, of one or more proteins. In some embodiments, a satiety modulating composition (e.g., a particle preparation) comprises one or more protein(s) in a core of a satiety modulating composition (e.g., a core-shell particle preparation). In some embodiments, a satiety modulating composition (e.g., a particle preparation) comprises one or more proteins in both a core and a shell of a satiety modulating composition (e.g., a core-shell particle preparation). In some embodiments, a satiety modulating composition (e.g., a particle preparation) comprises one or more protein(s) in a shell of a satiety modulating composition (e.g., a core-shell particle preparation). For example, one or more satiety modulator(s) includes L-glycine, L-alanine, L-arginine, L-asparagine, L-aspartic acid, L-cysteine, L-glutamine, L-glutamic acid, L-histidine, L-isoleucine, L-leucine, L-lysine, L-methionine, L-phenylalanine, L-proline, L-serine, L-threonine, L-tryptophan, L-tyrosine, and/or L-valine, amylase, avenin, calcium caseinate, cellulase, collagen, corn protein isolate, fibroin, gelatin, glutanin, kefirin, lipase, milk protein concentrate, oat protein isolate, pea protein isolate, protease, rice protein isolate, sodium caseinate, soy protein isolate, wheat protein isolate, whey protein isolate, zein, or any combination thereof.

In some embodiments, a protein is extracted from natural sources and substantially modified and/or purified. In some embodiments, an extracted protein is substantially modified, for example, truncated, hydrolyzed, complexed, digested, or any combination thereof. In some embodiments, a protein comprises a full-length, an unmodified, a truncated, a hydrolysates, a complex, and/or a digest of L-glycine, L-alanine, L-arginine, L-asparagine, L-aspartic acid, L-cysteine, L-glutamine, L-glutamic acid, L-histidine, L-isoleucine, L-leucine, L-lysine, L-methionine, L-phenylalanine, L-proline, L-serine, L-threonine, L-tryptophan, L-tyrosine, and/or L-valine, amylase, avenin, calcium caseinate, cellulase, collagen, corn protein isolate, fibroin, gelatin, glutanin, kefirin, lipase, milk protein concentrate, oat protein isolate, pea protein isolate, protease, rice protein isolate, sodium caseinate, soy protein isolate, wheat protein isolate, whey protein isolate, zein, or any combination thereof.

In certain embodiments, a protein is extracted from one or more natural sources and is compositionally well-defined. In certain embodiments, a protein is extracted from one or more natural sources and is compositionally poorly defined. In some embodiments, a compositional definition and/or lack of definition contributes to the efficacy of one or more protein(s) as a satiety modulator. In some embodiments, a compositional definition of one or more protein(s), one or more lipid(s), one or more carbohydrate(s), and/or one or more acaloric phytonutrient(s) is modulated by an enzymatic activity of one or more protein satiety modulator(s). In some embodiments, a compositional definition of one or more protein(s), one or more lipid(s), one or more carbohydrate(s), and/or one or more acaloric phytonutrient(s) contributes to the efficacy of one or more satiety modulating composition(s).

In certain embodiments, one or more satiety modulator(s) comprises a carbohydrate. In some embodiments, one or more carbohydrates comprise one or more D-monosaccharides chemically bonded together via one or more O-, N-, S-, and/or C-glycosidic linkages as disaccharides, trisaccharides, oligosaccharides, and/or polysaccharides. In some embodiments, one or more satiety modulator(s) comprise a monomeric D-monosaccharide. In some embodiments, a carbohydrate may be have a molecular weight of at least about 50 Da, about 200 Da, about 500 Da, about 1000 Da, about 5000 Da, about 20000 Da, about 100000 Da, about 500000 Da, about 1000000 Da, and/or about 5000000 Da. In some embodiments, a carbohydrate may be naturally derived (e.g., synthesized in nature and chemically purified), synthetically derived (e.g., synthesized by man and chemically purified), and/or semi-synthetically derived (e.g., synthesized in nature and further derivatized by man, followed by chemical purification). In some embodiments, as described herein, one or more carbohydrates may b increase the viscosity of an aqueous medium.

In some embodiments, a satiety modulating composition (e.g., a particle preparation) comprises about 10% to about 30% (e.g., about 10% to about 30%, about 15% to about 30%, about 20% to about 30%, about 25% to about 30%, about 10% to about 25%, about 15% to about 25%, about 20% to about 25%, about 10% to about 20%, about 15% to about 20%, or about 10% to about 15%), on a dry weight basis, of one or more carbohydrates. In some embodiments, a satiety modulating composition (e.g., a particle preparation) comprises one or more carbohydrates in a core of the satiety modulating composition (e.g., a core-shell particle preparation). In some embodiments, a satiety modulating composition (e.g., a particle preparation) comprises one or more carbohydrates in both a core and a shell of a satiety modulating composition (e.g., a core-shell particle preparation). In some embodiments, a satiety modulating composition (e.g., a particle preparation) comprises one or more carbohydrates in a shell of a satiety modulating composition (e.g., a core-shell particle preparation)

For example, in some embodiments, one or more satiety modulator(s) comprises D-glucose, D-galactose, D-fructose, maltose, D-xylose, D-mannose, sucrose, isomaltulose, trehalose, D-psicose, tagatose, lactose, lactulose, arabinose, amylopectin, dextran, pectin, amylose, inulin, locust bean gum, maltodextrin, xanthan gum, gum arabic, karaya gum, ghatti gum, guar gum, sodium carboxymethylcellulose, sodium alginate, sodium hyaluronate, calcium alginate, agarose, chitosan, chitin, carrageenan, chondroitin sulfate, hydroxypropyl methylcellulose, methyl cellulose, modified cellulose gum, ethyl cellulose, hydroxyethylcellulose, corn starch, cellulose triacetate, cellulose acetate butyrate, cellulose, cellulose acetate propionate, cellulose acetate succinate, cellulose acetate phthalate, hydroxypropyl methylcellulose acetate succinate, or any combination thereof.

In certain embodiments, a carbohydrate may be extracted from natural sources and substantially modified and/or purified. In some embodiments, an extracted carbohydrate is modified (e.g., truncated, hydrolyzed, complexed, and/or digested). For example, in some embodiments one or more satiety modulator(s) includes full-length, unmodified, truncated, hydrolyzed, complexed, and/or digested D-glucose, D-galactose, D-fructose, maltose, D-xylose, D-mannosc, sucrose, isomaltulose, trehalose, D-psicosc, tagatose, lactose, lactulose, arabinose, amylopectin, dextran, pectin, amylose, inulin, locust bean gum, maltodextrin, xanthan gum, gum arabic, karaya gum, ghatti gum, guar gum, sodium carboxymethylcellulose, sodium alginate, sodium hyaluronate, calcium alginate, agarose, chitosan, chitin, carrageenan, chondroitin sulfate, hydroxypropyl methylcellulose, methyl cellulose, modified cellulose gum, ethyl cellulose, hydroxyethylcellulose, corn starch, cellulose triacetate, cellulose acetate butyrate, cellulose, cellulose acetate propionate, cellulose acetate succinate, cellulose acetate phthalate, hydroxypropyl methylcellulose acetate succinate, or any combination thereof.

In certain embodiments, one or more satiety modulator(s) comprises an acaloric phytonutrient. In certain embodiments, one or more acaloric phytonutrient(s) is extracted from one or more plant(s) without substantial modification. In certain embodiments, one or more acaloric phytonutrient(s) is characterized as providing no caloric value to one or more mammal(s). In some embodiments, one or more acaloric phytonutrient(s) are components of whole vegetables, fruits, legumes, beans, roots, and/or shoots.

In some embodiments, a satiety modulating composition (e.g., a particle preparation) comprises about 5% to about 15% (e.g., about 5% to about 15%, about 7.5% to about 15%, about 10% to about 15%, about 12.5% to about 15%, about 5% to about 12.5%, about 7.5% to about 12.5%, about 10% to about 12.5%, about 5% to about 10%, about 7.5% to about 10%, or about 5% to about 7.5%), on a dry weight basis, of one or more acaloric phytonutrient. In some embodiments, a satiety modulating composition (e.g., a particle preparation) comprises one or more acaloric phytonutrient in a core of the satiety modulating composition (e.g., a core-shell particle preparation). In some embodiments, a satiety modulating composition (e.g., a particle preparation) comprises one or more acaloric phytonutrient in both a core and a shell of the satiety modulating composition (e.g., a core-shell particle preparation). In some embodiments, a satiety modulating composition (e.g., a particle preparation) comprises one or more acaloric phytonutrient in a shell of the satiety modulating composition (e.g., a core-shell particle preparation).

In certain embodiments, one or more satiety modulator(s) includes a flavonoid, a carotenoid, an iridoid, or a alkylresorcinol (e.g., cannabinoid). In some embodiments, an acaloric phytonutrient may be a partial agonist, a full agonist, a partial antagonist, a full antagonist, a partial allosteric modulator, or a full allosteric modulator of at least one of GPRC6A, CaSR, TasteR, GPR93, FFAR2, FFAR3, FFAR1, FFAR4, GPR40. GPR119, CB1, GLP-1R, GLP-2R, GIPR, IR, Y2R, and/or GPR120. In some embodiments, an acaloric phytonutrient is a satiety modulator independent of the presence of other satiety modulator(s). In some embodiments, one or more acaloric phytonutrient(s) enhance the effect of one or more other satiety modulator(s), for example, one or more lipid(s), one or more protein(s), one or more carbohydrate(s), and/or one or more acaloric phytonutrient(s).

In some embodiments, an acaloric phytonutrient includes quercetin, kaempferol, myricetin, fisetin, rutin, isorhamnetin, naringenin, silybin, eriodictyol, apigenin, chrysin, delphinidin, betanin, cyanidin chloride, neohesperidin, epigallocatechin, diosmetin, baicalein, genistein, oleuropein, amarogentin, genipin, aucubin, catalpol, olivetol, cannabidiol, tetrahydrocannabinol, daidzein, pelargonidin, tangeritin, luteolin, wogonin, epicatechin, catechin, theaflavin, resveratrol, hydroxytyrosol, and or any combination thereof.

### 2. Spatial Arrangement of Satiety Modulators

In certain embodiments of the present disclosure, one or more spatial arrangement(s) of one or more satiety modulator(s) is provided. Without wishing to be bound by any particular theory, in some embodiments, it is contemplated that a spatial arrangement of one or more satiety modulator(s) contributes to the utility of a satiety modulating composition disclosed herein.

In certain embodiments of the present disclosure, a satiety modulating composition (e.g., a particle preparation) includes one or more spatial arrangement(s) of one or more satiety modulator(s). In some embodiments, a particle preparation refers to one or more particles, together comprising one or more satiety modulator(s). In some embodiments, each particle comprises the same satiety modulator(s) in substantially similar quantity, and/or in substantially similar spatial arrangement. In some embodiments, one or more particles in a preparation are nonidentical and may comprise one or more different satiety modulators, may comprise one or more different satiety modulators in different quantities, and/or may comprise one or more different satiety modulators in different spatial arrangements. In some embodiments, a particle may be substantially spherical. In some embodiments, a particle may be prism-shaped.

### (i) Particle Preparations

In some embodiments, one or more satiety modulating composition(s) are or comprise particles (e.g., particle preparations). In some embodiments, the present disclosure provides particle preparations in which particles have a particular shape or form, for example, having a cross-sectional shape of a circle, an oval, a triangle, a square, a hexagon, or an irregular shape. In some embodiments, a preparation includes particles of different shapes or forms. In some embodiments, most or substantially all or all particles in a preparation have a common shape.

In some embodiments, particles in a provided particle preparation may have a distribution of diameters (e.g., Dv(10), Dv(20), Dv(30), Dv(40), Dv(50), Dv(60), Dv(70), Dv(80), Dv(90), Dv(99), etc.). In some embodiments, particles in a provided particle preparation may have an average diameter (e.g., D[3,2], D[4,3], etc.). Regardless of the shape of the particle, the “diameter” (i.e., size) of a particle is the longest distance from one end of a particle to another end of the particle.

In some embodiments, particles in a particle preparation as described and/or utilized herein may have a distribution of diameters (e.g., Dv(10), Dv(20), Dv(30), Dv(40), Dv(50), Dv(60), Dv(70), Dv(80), Dv(90), Dv(99), etc.) of up to about 10000 μm, up to about 5000 μm, up to about 2500 μm, up to about 1250 μm, up to about 800 μm, up to about 400 μm, up to about 200 μm, up to about 100 μm, up to about 50 μm, up to about 40 μm, up to about 30 μm, up to about 20 μm, up to about 10 μm, or up to about 5 μm.

Without wishing to be bound by any particular theory, it is contemplated that a spatial arrangement of one or more satiety modulating composition(s) as a particle preparation is advantageous towards incorporation of the one or more satiety modulating composition(s) into food and/or beverage product(s) so as to minimize changes to taste and/or texture. Without wishing to be bound by any particular theory, in some embodiments, a spatial arrangement disclosed herein is advantageous towards preparing one or more satiety modulating composition(s) characterized as having complex release behaviors. Without wishing to be bound by any particular theory, in some embodiments, a spatial arrangement disclosed herein is advantageous towards uniform release in a biological environment.

### (ii) Core-Shell Particle Preparations

Among other things, the present disclosure provides one or more satiety modulating composition(s) characterized as core-shell particle preparations. In some embodiments, a core-shell particle preparation may comprise a core (e.g., an interior component). In some embodiments, a core may include one or more satiety modulator(s). In some embodiments, a core-shell particle preparation may comprise one or more shells (e.g., coatings, exterior components). In some embodiments, one or more shells may include one or more satiety modulator(s).

In some embodiments, a satiety modulating composition (e.g. a core-shell particle preparation) includes a core comprising one or more lipids, one or more proteins, one or more carbohydrates, one or more acaloric phytonutrients, or any combination thereof. In some embodiments, a satiety modulating composition (e.g. a core-shell particle preparation) includes one or more shells comprising one or more lipids, one or more proteins, one or more carbohydrates, one or more acaloric phytonutrients, or any combination thereof. In some embodiments, a satiety modulating composition (e.g. a core-shell particle preparation) includes a core and one or more shells, where both the core and one or more shells comprise one or more lipids, one or more proteins, one or more carbohydrates, one or more acaloric phytonutrients, or any combination thereof.

In some embodiments, a satiety modulating composition (e.g., core-shell particle preparation) comprises a core encapsulated by 2-15, 2-10, 2-8, 2-6, 2-4, or 2-3 shells. In some embodiments, a satiety modulating composition (e.g., core-shell particle preparation) comprises a core encapsulated by 2 shells.

In some embodiments, a core is characterized as being a liquid. In some embodiments, a core is characterized as being a solid. In some embodiments, one or more shells are characterized as being a liquid. In some embodiments, one or more shells are characterized as being a solid.

In some embodiments, core-shell particle preparations of the present disclosure may be an emulsion, a suspension, a powder, a bar, a gel, a capsule, a tablet, a fiber, an extrudate, a hard candy, a chip, or a mesh.

For example, in some embodiments, a core-shell particle preparation disclosed herein may be an emulsion. In certain embodiments, an emulsion disclosed herein may be characterized as having low solubility (e.g., miscibility) in water. In some embodiments, an emulsion may be characterized as being amphiphilic. In certain embodiments, a core-shell particle preparation disclosed herein includes a core characterized as having low solubility (e.g., miscibility) in water and two or more shells characterized as being amphiphilic. In certain embodiments, a core-shell particle preparation disclosed herein includes a core characterized as having high solubility (e.g., miscibility) in water and two or more shells characterized as being amphiphilic.

In certain embodiments, a core-shell particle preparation includes a solid core. In some embodiments, a core-shell particle preparation includes one or more solid shells. In some embodiments, a solid core and/or a solid shell is characterized as having a melting point greater than 20° C. In some embodiments, a core includes a solid blend and/or gel comprising one or more satiety modulator(s). In some embodiments, one or more shells include a solid blend, gel, and/or film comprising one or more satiety modulator(s). In some embodiments, one or more shells have a thickness of at least about 1 nm, about 10 nm, about 100 nm, about 1 μm, about 10 μm, or about 100 m. In some embodiments, one or more shells and/or core is insoluble in water and one or more edible oils.

In certain embodiments, a core-shell particle preparation disclosed herein is characterized as controlling the release of one or more satiety modulator(s). In certain embodiments, two or more shells control the release of one or more satiety modulator(s) in a core of the core-shell particle preparation. In certain embodiments, a core controls the release of one or more satiety modulator(s) in two or more shells. In certain embodiments, two or more shells provide a physical barrier to an environment. In some embodiments, two or more shells control: diffusion of one or more satiety modulator(s) from a core-shell particle preparation; one or more chemical properties of a core of a core-shell particle preparation; a residence time of a core in an environment; responsiveness to one or more physical and/or chemical triggers, or any combination thereof. In certain embodiments, a core of a core-shell particle preparation controls: diffusion of one ore more satiety modulator(s) from the core-shell particle preparation; chemical properties of two or more shells of the core-shell particle preparation, a residence time of two or more shells in a dissolution medium; responsiveness to one or more physical and/or chemical triggers, or any combination thereof.

In certain embodiments, two or more shells provide a physical barrier between an environment and a core of a core-shell particle preparation disclosed herein. For example, in some embodiments, two or more shells may be insoluble in aqueous media (e.g., water, phosphate buffered saline solution, simulated intestinal fluid, simulated gastric fluid, simulated tear fluid, simulated urine, HEPES buffered saline solution, Dulbecco's Modified Eagle Medium, Hank's balanced salt solution, biological intestinal fluid, biological gastric fluid, plasma, saliva, urine, feces, sweat, tear fluid, and/or Kreb's buffer). In some embodiments, two or more shells may be characterized as having slow or zero-order solubilization in aqueous media. Without wishing to be bound by any particular theory, it is contemplated that two or more shells prevent access of an aqueous media to a core of a core-shell particle preparation, thus preventing release of one or more satiety modulators within the core.

### 3. Properties Satiety Modulating Compositions Affecting Release

In certain embodiments, a spatial arrangement of one or more satiety modulator(s) determines their respective release into an environment. In some embodiments, one or more chemical and/or physical properties of one or more satiety modulator(s), determines their respective release into an environment.

In certain embodiments, one or more satiety modulator(s) may be characterized as conferring chemical and/or physical protection, solidification, responsiveness, retention, and/or bioavailability to one or more satiety modulating composition(s).

### (1) Protection

In some embodiments, one or more satiety modulator(s) are characterized as being able to confer chemical and/or physical protection to one or more satiety modulating composition(s). In some embodiments, such chemical and/or physical protection extends the stability of one or more satiety modulating composition(s) within one or more food and/or beverage product(s). In some embodiments, such chemical and/or physical protection extends the stability of one or more satiety modulating composition(s) within one or more biological environments. In some embodiments, such chemical and/or physical protection reduces an effective dose of one or more satiety modulating composition(s). In some embodiments, such chemical and/or physical protection mitigates chemical and/or physical instability of one or more satiety modulator(s) prior to release in a certain biological compartment of a mammal.

In some embodiments, stability of one or more satiety modulator(s) in one or more satiety modulating composition(s) can be characterized as the percentage of change of a measured stability characteristic (e.g., stability property) after a period of storage relative to the stability characteristic immediately after formulation. In some embodiments, a change in one or more measured stability characteristic of one or more satiety modulator(s) is less than about 40%, less than about 30%, less than about 20%, less than about 10%, less than about 5%, less than about 2%, and/or less than about 1% after a period of storage relative to the stability characteristic immediately after formulation. In some embodiments, a measured stability characteristic includes a chemical stability of one or more satiety modulator(s). In some embodiments, a chemical stability may be characterized as a chemical quantity (e.g., mol, g, lbs) of one or more satiety modulator(s) after a period of storage relative to a chemical quantity immediately after formulation. In some embodiments, a stability characteristic includes a physical stability of one or more satiety modulator(s). In some embodiments, a physical stability may be characterized as a physical quantity (e.g., diameter, morphology, porosity) of one or more satiety modulator(s) after a period of storage relative to a physical quantity immediately after formulation. In some embodiments, a measured stability characteristic includes a functional stability of one or more satiety modulator(s). In some embodiments, a functional stability may be characterized as a release profile of one or more satiety modulator(s) after a period of storage relative to a release profile immediately after formulation. In some embodiments, a stability characteristic includes a stability of one or more physiological benefit conferred by one or more satiety modulator(s) in a mammal. In some embodiments, stability of one or more physiological benefit may be characterized as maintenance of health, maintenance of microbiome health, provision of energy, provision of metabolic intermediates, and/or provision of osmotic stability of one or more satiety modulator(s) in a mammal after a period of storage relative to a physiological benefit immediately after formulation.

For example, as provided herein, one or more satiety modulator(s) confer protection to one or more satiety modulating composition(s) by inhibiting chemical oxidation, inhibiting chemical reduction, inhibiting chemical degradation, inhibiting proteolysis in a biological environment, inhibiting lipolysis in a biological environment, inhibiting release of one or more satiety modulator(s), inhibiting hydrolysis in a biological environment, or any combination thereof.

### (ii) Solidification

In some embodiments, one or more satiety modulator(s) may be characterized by an ability to confer solidification to one or more satiety modulating composition(s). In some embodiments, solidification of one or more satiety modulating composition(s) confers stability during physical processing. For example, in some embodiments, solid satiety modulating composition(s) retain a predetermined spatial arrangement when exposed to pressure and shear forces routinely encountered during food processing. For example, in some embodiments, solid satiety modulating composition(s) retain a predetermined spatial arrangement when exposed to pressure and shear forces routinely encountered within a biological environment, during chemical digestion, and/or during mechanical digestion. In some embodiments, solidification reduces an effective dose of one or more satiety modulating composition(s). In certain embodiments, solidification mitigates physical instability of one or more satiety modulating composition(s) prior to release in a certain biological compartment.

For example, in some embodiments, one or more satiety modulator(s) may be characterized as conferring solidification to a core and/or one or more shell of a core-shell particle preparation disclosed herein by inducing gelation via cross-linking, inducing gelation via hydrogen-bonding, increasing molecular order to raise the melting point, or a combination thereof.

(iii) Responsiveness

In some embodiments, one or more satiety modulating composition(s) disclosed herein are characterized as having controlled release of one or more satiety modulator(s) that is responsive to one or more triggers. In certain embodiments, responsiveness of one or more satiety modulating composition is characterized as a physical and/or chemical change to one or more triggers.

In some embodiments, responsiveness of a satiety modulating composition is characterized as a change in a physical property; for example, physical conformation, porosity, solubility, crystallinity, etc. In some embodiments, responsiveness of a satiety modulating composition is characterized as a change in a chemical property; for example, molecular weight, formation and/or cleavage of a chemical bond, ionization and/or charge, glass transition temperature, etc. In some embodiments, responsiveness of a satiety modulating composition is characterized as a change in both physical and chemical properties.

In some embodiments, responsiveness extends the stability of one or more satiety modulating composition(s) within one or more food and/or beverage product(s). In some embodiments, responsiveness extends the stability of one or more satiety modulating composition(s) within one or more biological environments. In some embodiments, responsiveness reduces an effective dose of one or more satiety modulating composition(s). In certain embodiments, responsiveness mitigates chemical and/or physical instability of one or more satiety modulating composition(s) prior to release in a certain biological compartment.

In some embodiments, one or more satiety modulating compositions are responsive to pH (e.g., pH-responsive), temperature (e.g., temperature-responsive), light (e.g., light-responsive), water (e.g., water-responsive), pressure (e.g., mechano-responsive), enzymes (e.g., chemo-responsive), and/or living organisms (e.g., bio-responsive).

### (iv) Retention

In some embodiments, one or more satiety modulator(s), oleogelator(s), pH-responsive polysaccharide(s), or any combination thereof, are characterized as facilitating retention of one or more satiety modulating composition(s) in one or more biological compartments. In some embodiments, retention extends a duration that one or more biological compartment(s) is exposed to one or more satiety modulator(s). In some embodiments, retention of one or more satiety modulating composition(s) prolongs biological exposure to one or more satiety modulator(s) for at least about 2 hours, at least about 4 hours, at least about 6 hours, at least about 8 hours, at least about 12 hours, at least about 16 hours, and/or at least about 24 hours. In some embodiments, retention reduces an effective dose of one or more satiety modulating composition(s).

In some embodiments, retention is characterized as the interaction (e.g., binding, bonding, adhesion) of one or more satiety modulating composition(s) with one or more biological compartments. In some embodiments, retention is characterized as occlusion of one or more satiety modulating composition(s) in one or more biological compartments. In some embodiments, retention is characterized as buoyancy (e.g., resistance to peristalsis) in one or more biological compartments.

### (v) Bioavailability

In some embodiments, one or more satiety modulating composition(s) regulates the bioavailability of one or more satiety modulator(s). In some embodiments, bioavailability is characterized as a total amount of a single orally administered dose of one or more satiety modulator(s) appearing in the systemic plasma circulation of one or more mammal(s) expressed as a fraction (e.g., percent) of the total amount of one or more satiety modulator(s) appearing in the systemic plasma circulation of one or more mammal(s) following a single intravenous dose. In some embodiments, an increase in bioavailability reduces an effective dose of one or more satiety modulating composition(s).

Without wishing to be bound by any particular theory, it is contemplated that one or more satiety modulating compositions increase bioavailability of one or more satiety modulators by enhancing bioadhesion, disrupting cell membranes, increasing endocytosis, opening enterocytic tight junctions, or any combination thereof.

4. Release of One or More Satiety Modulators from One or More Satiety Modulating Compositions

In certain embodiments of the provided disclosure, one or more satiety modulator(s) is released from one or more satiety modulating composition(s) (e.g., particle preparations). In some embodiments, release of one or more satiety modulator(s) is characterized by the transfer of one or more satiety modulator(s) from one or more satiety modulating composition(s) to a surrounding environment. In some embodiments, release of one or more satiety modulator(s) is characterized by an increase in the concentration of one or more satiety modulator(s) in a surrounding environment. Additionally, or alternatively, a quantity of one or more satiety modulator(s) within one or more satiety modulating composition(s) (e.g., particle preparations) decrease as the one or more satiety modulator(s) are released into a surrounding environment.

In certain embodiments, the release of one or more satiety modulator(s) is expressed as a total mass of one or more satiety modulator(s) present in a release environment. In certain embodiments, the release of one or more satiety modulator(s) is expressed as a fraction of total mass of one or more satiety modulator(s) present in a release environment relative to total mass of one or more satiety modulator(s) initially present (e.g., prior to release) in one or more satiety modulating composition(s) (e.g., percent release). In certain embodiments, the release of one or more satiety modulator(s) is expressed as a fraction of total mass of one or more satiety modulator(s) remaining in one or more satiety modulating composition(s) relative to total mass of one or more satiety modulator(s) initially present in one or more satiety modulating composition(s). In certain embodiments, the release of one or more satiety modulator(s) is expressed as a function of sampling time (e.g., release kinetics). In certain embodiments, a release rate is determined from modeling and/or derivatization of the release kinetics of one or more satiety modulator(s) from one or more satiety modulating composition(s).

In some embodiments, a release profile (e.g., release kinetics, release rate) of one or more satiety modulating composition(s) is specific to a certain release environment.

### (i) Release Environment

In some embodiments, a release environment includes a liquid, solid, or gaseous medium in which one or more satiety modulating composition(s) is dispersed. In some embodiments, a release environment includes a food and/or beverage product prior to consumption by a mammal. In some embodiments, a release environment includes a biological compartment (e.g., mouth, stomach, duodenum, jejunum, ileum, cecum, colon, or rectum). In some embodiments, a release environment includes an in vitro laboratory apparatus used to approximate a biological compartment (e.g., a 15 mL polypropylene centrifuge tube comprising aqueous solution, a USP Type II dissolution apparatus, etc.).

In some embodiments, release of one or more satiety modulator(s) includes dispersal and/or biological transit (e.g., peristalsis) in several successive release environments. For example, in some embodiments, a release environment is or may be a gastrointestinal tract of one or more mammal(s), where one or more satiety modulating composition(s) (e.g., particle preparations) are dispersed within at least one of a buccal cavity, an esophagus, a stomach, a jejunum, a duodenum, an ileum, a colon etc.

### (ii) Controlled Release

In certain embodiments, controlled release is release of one or more satiety modulator(s) from one or more satiety modulating composition(s) (e.g. particle preparations) occurring in at least one of a certain biological compartment, a certain pH, a certain temperature, after a certain period of time (e.g., storage), a certain shear rate, exposure to one or more enzyme(s) and/or bacteria, exposure to light, exposure to water, or any combination thereof. In certain embodiments, the physical and/or chemical properties of one or more satiety modulator(s) contributes to the controlled release of the one or more satiety modulator(s) from one or more satiety modulating composition(s). In certain embodiments, the spatial arrangement of one or more satiety modulator(s) contributes to the controlled release of the one or more satiety modulator(s) from one or more satiety modulating composition(s).

(iii) In-Vitro Release Profile

In certain embodiments, the release of one or more satiety modulator(s) is quantified (e.g., release profile, release kinetics) in an in vitro release environment (i.e., in vitro release) to approximate the release in one or more biological environment(s). In certain embodiments, the release of one or more satiety modulator(s) is quantified (e.g., release profile, release kinetics) in an in vitro release environment to approximate release in one or more food and/or beverage product environment(s). In some embodiments, one or more in vitro release profiles of one or more satiety modulating composition(s) (e.g., particle preparations) is determined by quantifying the percent release of one or more satiety modulator(s) into a release environment.

### (iv) In Vivo Release Profile

In certain embodiments, the release of one or more satiety modulator(s) is quantified (e.g., release profile, release kinetics) in a biological release environment (e.g., in vivo release). In some embodiments, one or more in vivo release profiles of one or more satiety modulating composition(s) is determined by quantifying the bioavailability of one or more satiety modulator(s).

### 5. Water Activity

Without wishing to be bound by any particular theory, one or more satiety modulator(s) may exhibit poor stability in environments with high water activity. In certain embodiments of the present disclosure, one or more satiety modulating(s) are characterized as having a water activity of less than about 0.4, less than about 0.3, less than about 0.2, or less than about 0.1.

In certain embodiments, one or more satiety modulating composition(s) are characterized as having a water activity of less than about 0.4, less than about 0.3, less than about 0.2, or less than about 0.1.

Without wishing to be bound by any particular theory, technologies disclosed herein having low water activities provide benefits over existing products having high water activities because high water activity formulations lead to rapid degradation of satiety modulator(s).

### 6. Effective Dose of One or More Satiety Modulators

In certain embodiments of the present disclosure, one or more satiety modulating composition(s) include an effective dose of one or more satiety modulator(s). In some embodiments, an effective dose of one or more satiety modulator(s) is characterized as being a quantity sufficient to elicit a satiety response in one or more mammal(s). In certain embodiments, an effective dose of one or more satiety modulator(s) that is effective for one mammal is effective for one or more other mammals. In certain embodiments, an effective dose of one or more satiety modulator(s) that is effective for one mammal is ineffective for one or more other mammals. In certain embodiments, an effective dose of one or more satiety modulator(s) is scalable between two or more mammals relative to total body surface area. In certain embodiments, an effective dose of one or more satiety modulator(s) is determined by measuring a satiety response exhibited by one or more mammal(s).

### (i) Mass of One or More Satiety Modulator(s)

In some embodiments, an effective dose of one or more satiety modulators is about 0.1 g to about 100 g, about 0.5 g to about 100 g, about 1 g to about 100 g, about 5 g to about 100 g, about 10 g to about 100 g, about 50 g to about 100 g, about 0.1 g to about 50 g, about 0.5 g to about 50 g, about 1 g to about 50 g, about 5 g to about 50 g, about 10 g to about 50 g, about 0.1 g to about 10 g, about 0.5 g to about 10 g, about 1 g to about 10 g, about 5 g to about 10 g, about 0.1 g to about 5 g, about 0.5 g to about 5 g, about 1 g to about 5 g, about 0.1 g to about 1 g, about 0.5 g to about 1 g, or about 0.1 g to about 0.5 g. In some embodiments, an effective dose of one or more satiety modulator(s) is at least about 0.1 g, at least about 0.5 g, at least about 1 g, at least about 5 g, at least about 10 g, at least about 50 g. or at least about 100 g. In some embodiments, an effective dose of one or more satiety modulator(s) is less than about 500 g.

### (ii) Caloric Content of One or More Satiety Modulators

In some embodiments, an effective dose of one or more satiety modulator(s) is about 0 kcal to about 250 kcal, about 50 kcal to about 250 kcal, about 100 kcal to about 250 kcal, about 150 kcal to about 250 kcal, about 200 kcal to about 250 kcal, about 0 kcal to about 200 kcal, about 50 kcal to about 200 kcal, about 100 kcal to about 200 kcal, about 150 kcal to about 200 kcal, about 0 kcal to about 150 kcal, about 50 kcal to about 150 kcal, about 100 kcal to about 150 kcal, about 0 kcal to about 100 kcal, about 50 kcal to about 100 kcal, or about 0 kcal to about 50 kcal. In some embodiments, an effective dose of one or more satiety modulator(s) is at least about 0 kcal, at least about 5 kcal, at least about 20 kcal, at least about 50 kcal, at least about 100 kcal, and/or at least about 250 kcal. In some embodiments, an effective dose of one or more satiety modulator(s) is less than about 500 kcal.

7. Incorporation of Satiety Modulating Compositions into Food and/or Beverage Products

In certain embodiments, the current disclosure provides for the incorporation of one or more satiety modulating composition(s) (e.g., particle preparations) into food and/or beverage products.

In some embodiments, one or more satiety modulating composition(s) are incorporated into food and/or beverage products in a food and/or beverage manufacturing process. In some embodiments, one or more satiety modulating composition(s) are incorporated into food and/or beverage products in a food and/or beverage packaging process. In some embodiments, one or more satiety modulating composition(s) are incorporated prior to pasteurization of a food and/or beverage product. In some embodiments, one or more satiety modulating composition(s) are incorporated prior to mixing of a food and/or beverage product. In some embodiments, one or more satiety modulating composition(s) are incorporated into finished food and/or beverage products. In some embodiments, one or more satiety modulating composition(s) are incorporated into food and/or beverage products immediately prior to consumption.

In certain embodiments, incorporation of satiety modulating composition(s) into food and/or beverage products utilize size reduction techniques and/or homogenization. In some embodiments, size reduction techniques are applied to satiety modulating composition(s) prior to incorporation into food and/or beverage products. In some embodiments, size reduction techniques are applied to food and/or beverage products during incorporation of satiety modulating composition(s) of the present disclosure. In some embodiments, size reduction techniques are applied to food and/or beverage products after incorporation of satiety modulating composition(s) disclosed herein. In some embodiments, size reduction techniques include planetary milling, ball milling, burr milling, roller milling, media milling, impact milling, jet milling, high-pressure homogenization, cryo milling, hammer milling, conical milling, hand screening, or granulation/extrusion, extrusion, spray drying, lyophilization/milling, fluid bed agglomeration, spray congealing, high-shear granulation, tableting, pouring, roller compaction, crosslinking, prilling, spinning disc atomization, of any combination thereof.

In certain embodiments, homogenization is applied to satiety modulating composition(s) following incorporation into food and/or beverage products. In some embodiments, homogenization includes using an overhead stirrer, manual stirring, using a stir bar, high pressure homogenization, low pressure homogenization, sonication, ultrasonication, vortexing, or combinations thereof.

In certain embodiments, incorporation of satiety modulating composition(s) into food and/or beverage products significantly affects the visual appearance, texture, and/or taste of the food and/or beverage products. In other embodiments, incorporation of satiety modulating composition(s) into food and/or beverage products minimally affects the visual appearance, texture, and/or taste of the food and/or beverage products.

In some embodiments, the present disclosure provides consumable compositions (e.g., food products, beverage products, animal-consumable compositions) comprising one or more satiety modulating compositions disclosed herein. In some embodiments, consumable compositions comprising one or more satiety modulating compositions disclosed herein include a food product. In some embodiments, a food product is characterized by high water activity. In some embodiments, a food product includes an agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, tablet, or yogurt. In some embodiments, consumable compositions comprising one or more satiety modulating compositions disclosed herein are provided to a mammal in a mixture with a food or food ingredient.

In some embodiments, consumable compositions comprising one or more satiety modulating compositions disclosed herein comprise a beverage product. In some embodiments, a beverage product is characterized by high water activity. In some embodiments, a beverage product includes a liquid supplement formulation, beer, seltzer, kefir, coffee, juice, liquid pharmaceutical formulation, milk, soda, sports drink (e.g., Gatorade, sports drinks, Vitamin beverage), tea, water, liquor (e.g., vodka, whiskey, rum, etc.) and/or wine. In some embodiments, one or more satiety modulating compositions (e.g., particle preparations) are provided to a mammal in a mixture with a beverage or beverage ingredient.

In some embodiments, the present disclosure provides a powder-based supplement, food, and/or beverage-mix products comprising one or more satiety modulating compositions disclosed herein. In some embodiments, a powder-based supplement, food, and/or beverage-mix products are characterized as having high water activity. In some embodiments, a powder-based supplement, food, and/or beverage-mix products include a pre-workout powder, post-workout powder or pill, pre-workout capsule/pill, baby formula, whey powder, milk powder, protein powder, or a drink powder mix (e.g., Kool-Aid type mix).

8. Stability of Satiety Modulators in Food and/or Beverage Products

In some embodiments, stability of one or more satiety modulator(s) in a provided satiety modulating composition is assessed over a period of time in a set of particular environmental conditions. In some embodiments, stability of one or more satiety modulator(s) is characterized as the maintenance of one or more physical and/or chemical properties; induction of satiety; solidification of one or more satiety modulating composition(s); maintenance of pH responsiveness; or a combination thereof, following a period of time, exposure to light, exposure to heat, exposure to moisture, exposure to mechanical forces, pH changes, or combination thereof in a certain non-biological environment.

In some embodiments, there is minimal change in one or more stability characteristic of one or more satiety modulating composition(s) disclosed herein after at least about 2 weeks, at least about 4, at least about 24 weeks, at least about 52 weeks, or at least about 260 weeks under a set of particular environmental conditions (e.g., ambient temperature). In some embodiments, one or more satiety modulating composition disclosed herein is characterized by a measured change in a physical, chemical, or functional property of less than about 40%, less than about 30%, less than about 20%, less than about 10%, less than about 5%, less than about 2%, or less than about 1% of a physical and/or chemical property, or functional property, of one or more satiety modulator(s) after at least about 2 weeks, at least about 4, at least about 24 weeks, at least about 52 weeks, or at least about 260 weeks under a set of particular environmental conditions.

In some embodiments, one or more satiety modulating compositions disclosed herein are stable (e.g., less than about 20% change in one or more stability characteristics) for at least about 2 weeks, at least about 1 month, at least about 1 year, or at least about 5 years in: a humid atmosphere (e.g., at least about 35% relative humidity, at least about 50% relative humidity, at least about 73% relative humidity, etc.), a cold temperature (e.g., 4° C., −20° C., −80° C.), a liquid beverage (e.g., coffee, drinkable yogurt, protein beverage, water, soda, Gatorade, sports drinks, etc.), a solid food (e.g., bread, rice, baked goods, etc.), a yogurt, a milk powder, a baby formula, a high fat dry powder, a sachet, and/or an animal feed (e.g., total meal ration, animal feed pellets, etc.) at ambient temperature.

### D. Methods of Controlling the Satiety of One or More Mammal(s)

In some embodiments, the present disclosure provides for a method of controlling satiety of one or more mammal(s) comprising a step of administering an effective dose of one or more satiety modulating composition(s). Without wishing to be bound by any particular theory, it is contemplated that the disclosed methods of controlling satiety of one or mammal(s) provide an integrated chemical sensory satiety input over a prolonged duration. In certain embodiments, the present disclosure provides methods of increasing the satiety of one or more mammal(s). In some embodiments, satiety is characterized as reducing a desire to consume and/or seek-out food and/or beverage products for a certain period of time. In certain embodiments, the present disclosure provides methods of decreasing the satiety of one or more mammal(s). In certain embodiments, methods of controlling satiety of one or more mammal(s) disclosed herein may be utilized for a therapeutic purpose (e.g., to provide for health). In certain embodiments, methods of controlling satiety of one or more mammal(s) disclosed herein may be utilized for hedonistic purpose (e.g., to provide and/or remove pleasure).

### 1. a Step of Administering an Effective Quantity of One or More Satiety Modulator(s)

In certain embodiments, a method of controlling the satiety of one or more mammal(s) comprises a step of administering an effective dose of one or more satiety modulating composition(s), as provided herein.

In certain embodiments, administering an effective dose of one or more satiety modulating composition(s) provides an integrated chemical satiety stimulus to one or more mammal(s). In certain embodiments, one or more satiety modulators released from one or more satiety modulating composition(s) may be recognized by cells present in a gastrointestinal tract of one or more mammal(s). In certain embodiments, one or more satiety modulating composition(s) provides a chemical stimulus to one or more mammal(s) through recognition by receptors present on one or more cell(s) in a gastrointestinal tract of one or more mammal(s). In some embodiments, such a chemical stimulus may elicit satiety in one or more mammal(s).

In certain embodiments, administration of one or more satiety modulating composition(s) is characterized by the protection of one or more satiety modulators, from degradation in a certain environment. In some embodiments, administration of one or more satiety modulating composition(s) is characterized by the protection of one or more satiety modulator(s) in a food and/or beverage product, or a pharmaceutical product. In some embodiments, administration of one or more satiety modulating composition(s) is characterized by the protection of one or more satiety modulator(s) in at least one of the buccal cavity, the esophagus, the stomach, the duodenum, the jejunum, the ileum, the cecum, the colon, and/or the rectum of one or more mammal(s). In certain embodiments, administration of one or more satiety modulating composition(s) is characterized by the protection of one or more satiety modulator(s) against enzymatic degradation, oxidation, reduction, hydrolysis, absorption, and/or mechanical debridement.

In certain embodiments, administration of one or more satiety modulating composition(s) is characterized by the release of one or more satiety modulator(s) in a certain biological environment. In some embodiments, at least about 75%, at least about 80%, at least about 85%, at least about 90%, or at least about 95% of one or more satiety modulators are released from one or more satiety modulating compositions disclosed herein in at least one of the buccal cavity, the esophagus, the stomach, the duodenum, the jejunum, the ileum, the cecum, the colon, and/or the rectum of one or more mammal(s). In some embodiments, administration of one or more satiety modulating composition(s) is characterized by the release of one or more satiety modulator(s) in both the upper and lower gastrointestinal tract. In certain embodiments, at least 40% of one or more satiety modulator(s) are released from one or more satiety modulating compositions disclosed herein in the upper gastrointestinal tract (e.g., buccal cavity, esophagus, stomach, and/or duodenum) and at least 50% of one or more satiety modulator(s) are released from one or more satiety modulating compositions disclosed herein in the lower gastrointestinal tract (e.g., jejunum, ileum, cecum, colon, and/or rectum).

In certain embodiments, one or more satiety modulating compositions disclosed herein releases one or more satiety modulates in response to pH (e.g., pH-responsive), temperature (e.g., temperature-responsive), light (e.g., light-responsive), water (e.g., water-responsive), pressure (e.g., mechano-responsive), enzymes (e.g., chemo-responsive), living organisms (e.g., bio-responsive), or any combination thereof.

In certain embodiments, one or more satiety modulating composition disclosed herein releases one or more satiety modulator over a certain period of time. For example, in some embodiments, administration of one or more satiety modulating composition(s) releases at least about 75%, at least about 80%, at least about 85%, at least about 90%, and/or at least about 95% of one or more satiety modulators after at least about 10 minutes, at least about 30 minutes, at least about 60 minutes, at least about 120 minutes, at least about 180 minutes, at least about 240 minutes, at least about 480 minutes, or at least about 960 minutes.

In certain embodiments, administration of one or more satiety modulating compositions (e.g. core-shell particle preparations) comprising a core and two or more shells to a mammal releases one or more satiety modulator(s) in an upper gastrointestinal tract of the mammal. In certain embodiments, administration of one or more satiety modulating compositions (e.g. core-shell particle preparations) comprising a core and two or more shells to a mammal releases one or more satiety modulator(s) in a lower gastrointestinal tract of the mammal.

### (i) Administration of One or More Satiety Modulating Compositions

In certain embodiments, an effective dose of one or more satiety modulating composition(s) is in the form of a pill, a crystal, an emulsion, an oil, a dispersion, a tablet, a gel-cap, a solution, a gel, a powder, a sachet, a bar, a granule, a particle preparation, a core-shell preparation, a capsule, a jelly, a suspension, an elixir, a syrup, a food, and/or a beverage. In embodiments, an effective dose of one or more satiety modulating composition(s) is or may be dispersed uniformly within a food and/or beverage product. In some embodiments, an effective dose of one or more satiety modulating composition(s) may be dispersed uniformly within one or more pharmaceutical composition(s).

In certain embodiments, an effective dose of one or more satiety modulating composition(s) is administered to one or more mammal(s) in order to control satiety. In certain embodiments, an effective dose of one or more satiety modulating composition(s) is administered to a mammal via an oral route. In certain embodiments, an effective dose of one or more satiety modulating composition(s) is administered to a mammal through a rectal route. In certain embodiments, an effective dose of one or more satiety modulating composition(s) is administered to a mammal through a percutaneous, subcutaneous, or intravenous route. In certain embodiments, one or more satiety modulating composition(s) is administered via consuming (e.g., eating, chewing, drinking, swallowing, etc.) one or more food and/or beverage products incorporated with an effective quantity of one or more satiety modulating composition(s). In certain embodiments, one or more satiety modulating compositions disclosed herein is administered via drinking a beverage product (e.g., fruit juice, soda, alcoholic beverage, Gatorade, vitamin mix) incorporating an effective amount of one or more satiety modulating composition(s). In certain embodiments, one or more satiety modulating composition(s) is administered via swallowing one or more pharmaceutical devices (e.g., tablet, pill, capsule, gel-cap, tincture, syrup, elixir, etc.) incorporating an effective dose of one or more satiety modulating composition(s).

### 2. Satiety

In certain embodiments of the present disclosure, one or more methods of controlling the satiety of one or more mammal(s) are provided. Without wishing to be bound by any particular theory, it is contemplated that controlling the satiety of one or more mammal(s) is of substantial benefit to the health and longevity of the one or more mammal(s) and/or to the satisfaction that one or more mammal(s) derives from food and/or beverage products.

In some embodiments, satiety may be associate with sensations of joy, euphoria, contentment, excitement, happiness, satisfaction, gratification, craving, dejection, worry, distress, sorrow, discomfort, pain, depression, fear, anxiety, nausea, confusion, or a combination thereof.

In certain embodiments, satiety is quantified by the presence and/or levels of one or more satiety hormone(s) produced by at least one of: a mammal, a mammalian cell line, a mammalian cancer cell line, or a mammalian organotypic intestinal model. In certain embodiments, modulation of satiety is quantified as a change in the presence and/or quantity of one or more satiety hormone(s) produced by at least one of: a mammal, a mammalian cell line, a mammalian cancer cell line, or a mammalian organotypic intestinal model. In certain embodiments, a quantity of one or more satiety hormone(s) is characterized by an amount of satiety hormone (e.g., protein), an amount of cell transcript (e.g., mRNA), enzymatic activity of a mammal, a mammalian healthy cell line, a mammalian cancer cell line, or a mammalian organotypic intestinal model, or a combination thereof. In some embodiments, satiety is quantified by levels of leptin, GLP-1, GLP-2, motilin, gastrin, insulin, ghrelin, peptide yy, cholecystokinin, GIP, serotonin, histamine, oleoylethanolamide, palmitoylethanolamide, anandamide, linolenoylethanolamide, their prehormones, their isoforms, their degradation products, their full-length transcripts, their spliced transcripts, or combinations thereof.

### (i) Methods of Quantifying Satiety

In certain embodiments of the present disclosure, one or more methods of controlling satiety in one or more mammal(s) includes induction of satiety. In certain embodiments, induction of satiety in one or more mammal(s) can be quantified (e.g., assigned a numerical value). In some embodiments, induction of satiety in one or more mammal(s) is qualified (e.g., assigned a subjective grading).

For example, in some embodiments, modulating satiety of one or more mammal(s) is quantified by measuring a quantity and/or a change in quantity of one or more satiety hormone(s). In certain embodiments, one or more biochemical techniques may be employed to quantify one or more satiety hormone(s); for example, ultraviolet absorption, visible absorption, infrared absorption, turbidity, intrinsic fluorescence, immunofluorescence, ELISA, circular dichroism, liquid chromatography, UV liquid chromatography, evaporative light scattering liquid chromatography, refractive index liquid chromatography, conductivity liquid chromatography, fluorescence liquid chromatography, mass spectrometry, gas chromatography, PCR, qPCR, western blot, aptamer-binding fluorescence, or any combination thereof.

For example, in some instances, satiety of one or more mammal(s) is qualified by a subjective grading of the state of emotion experienced by one or more mammal(s) prior to and/or following executing one or more method(s) of modulating satiety. In certain embodiments, qualification of emotional state is self-reported by one or more mammal(s). In certain embodiments, qualification of emotional state is determined by an external observer.

For example, in some embodiments, satiety of one or more mammal(s) is quantified by measuring the systemic absorption of one or more satiety modulator(s). In certain embodiments, one or more biochemical techniques are or may be employed to quantify one or more satiety modulator(s), for example, ultraviolet absorption, visible absorption, infrared absorption, turbidity, intrinsic fluorescence, immunofluorescence, ELISA, circular dichroism, liquid chromatography, UV liquid chromatography, evaporative light scattering liquid chromatography, refractive index liquid chromatography, conductivity liquid chromatography, fluorescence liquid chromatography, mass spectrometry, gas chromatography, PCR, qPCR, western blot, aptamer-binding fluorescence, or any combination thereof. In certain embodiments, increases in the systemic plasma concentration of one or more satiety modulator(s) indicates satiety control in one or more mammal(s). In certain embodiments, increases in the local (e.g., gastrointestinal) plasma concentration of one or more satiety modulator(s) indicates satiety control in one or more mammal(s). In certain embodiments, increases in the interstitial (e.g., tissue) concentration of one or more satiety modulator(s) indicates satiety control in one or more mammal(s).

### 3. Duration of Satiety Control

In certain embodiments, one or more methods of controlling the satiety of one or more mammal(s) is characterized as efficacious over a period of time following administration of one or more satiety modulating composition(s) (e.g., duration of effect).

As provided herein, one or more satiety modulating composition(s) is characterized as efficacious over a period of time following administration of one or more satiety modulating composition(s) to a mammal by quantifying the satiety induced in the mammal. In some embodiments, one or more satiety modulating composition(s) are characterized as effective by measuring the quantity and/or change in quantity of one or more satiety hormone(s), for example, leptin, GLP-1, GLP-2, motilin, gastrin, insulin, ghrelin, peptide yy, cholecystokinin, GIP, serotonin, histamine, oleoylethanolamide, palmitoylethanolamide, anandamide, linolenoylethanolamide, their prehormones, their isoforms, their degradation products, their full length transcripts, their spliced transcripts, or any combination thereof.

In some embodiments, one or more satiety modulating composition(s) is characterized as efficacious over a period of time following administration of one or more satiety modulating composition(s) by quantifying the release of one or more satiety modulator(s) in one or more biological compartment(s) after a certain period of time following administration. In some embodiments, one or more satiety modulating composition(s) is characterized as efficacious by quantifying released mass of one or more satiety modulator(s) and/or percent release of one or more satiety modulating composition(s).

Without wishing to be bound by any particular theory, it is contemplated that the provided methods of controlling satiety of one or more mammal(s) are particularly advantageous by providing satiety control over long durations (e.g., up to about 6, up to about 12, up to about 16, and/or up to about 24 hours). Without wishing to be bound by any particular theory, it is contemplated that extended duration of satiety control is of substantial benefit to the health and longevity of one or more mammal(s) and/or to the satisfaction that one or more mammal(s) derives from food and/or beverage products. For example, it is contemplated that providing a feeling of fullness for an extended duration (e.g., reducing a desire to eat) is particularly advantageous for those mammal(s) suffering from obesity and/or diabetes. For example, it is contemplated that providing a feeling of hunger (e.g., increasing a desire to eat) for an extended duration is particularly advantageous for those mammal(s) suffering from anorexia and/or sarcopenia.

In certain embodiments, provided method(s) of controlling satiety of one or more mammal(s) are characterized by a duration of effect on the quantity of one or more satiety hormone(s) in one or more mammal(s). As provided herein, a duration of effect is characterized by a change in quantity of one or more satiety hormone(s) of at least about 5%, at least about 10%, at least about 15%, at least about 20%, at least about 50%, at least about 75%, at least about 150%, and/or at least about 300%. In certain embodiments, one or methods of controlling the satiety of one or more mammal(s) is characterized by a change in one or more satiety hormone(s) over at least about 10 minutes, at least about 30 minutes, at least about 60 minutes, at least about 120 minutes, at least about 240 minutes, at least about 8 hours, at least about 12 hours, and/or at least about 24 hours following administration, relative to basal levels.

In certain embodiments, provided method(s) of controlling satiety of one or more mammal(s) are characterized by a duration of effect of release of one or more satiety modulator(s) in the gastrointestinal tract of one or more mammal(s). As provided herein, a duration of effect is characterized by a release of one or more satiety modulators(s) of at least about 5%, at least about 10%, at least about 15%, at least about 20%, at least about 50%, at least about 75%, at least about 90%, and/or at least about 95%. In certain embodiments, provided methods of controlling satiety of one or more mammal(s) is characterized by a release of one or more satiety modulator(s) over at least about 10 minutes, at least about 30 minutes, at least about 60 minutes, at least about 120 minutes, at least about 240 minutes, at least about 8 hours, at least about 12 hours, and/or at least about 24 hours following administration, relative to basal levels.

In certain embodiments, the selection and spatial arrangement (e.g., formulation) of one or more satiety modulating composition(s) provides for controlled satiety over extended durations (e.g., at least about 10 minutes, at least about 30 minutes, at least about 60 minutes, at least about 120 minutes, at least about 240 minutes, at least about 8 hours, at least about 12 hours, and/or at least about 24 hours). In certain embodiments, the quantity of administered satiety modulating composition(s) provides for controlled satiety over extended durations.

### VII. EXEMPLARY EMBODIMENTS

1. A non-therapeutic particle preparation comprising multi-layer core-shell spatial arrangement(s) of:
about 40% to about 75%, on a dry weight basis, of one or more lipid(s),
about 10% to about 30%, on a dry weight basis, of one or more protein(s),
about 10% to about 30%, on a dry weight basis, of one or more carbohydrate(s),
and about 5% to about 15%, on a dry weight basis, of one or more acaloric phytonutrient(s)
wherein at least 90% of the at least one lipid, at least one protein, at least one carbohydrate, and/or at least one acaloric phytonutrient are released in both upper gastrointestinal and lower gastrointestinal region(s) of one or more mammal(s).
2. The composition of any of embodiment 1, wherein the particle preparation comprises at least 1, at least 10, at least 100, at least 1000, at least 10000, at least 100000, and/or at least 1000000 particles.
3. The composition of embodiment 2, wherein the particle preparation comprises identical multi-layer core-shell spatial arrangements of the at least one lipid, at least one protein, at least one carbohydrate, and/or at least one acaloric phytonutrient(s).
4. The composition of embodiment 2, wherein the particle preparation comprises different multi-layer core-shell spatial arrangements of the at least one lipid, at least one protein, at least one carbohydrate, and/or at least one acaloric phytonutrient(s).
5. The composition of embodiment 1, wherein the average diameter of the particle preparation is at least about 1 μm, at least about 10 μm, and/or at least about 20 m as determined by laser diffractometry.
6. The composition of embodiment 5, wherein the average diameter of the particle preparation is no more than about 50 μm as determined by laser diffractometry.
7. The composition of embodiment 1, wherein the particle preparation comprises a core-shell structure of at least one core and at least two shells.
8. The composition of embodiment 7, wherein the particle preparation comprises a mononuclear core-shell structure.
9. The composition of embodiment 7, wherein the particle preparation comprises a polynuclear core-shell structure.
10. The composition of embodiment 7, wherein the at least one core and at least two shells are solid.
11. The composition of embodiment 10, wherein the at least one core and at least two shells each possess a melting point greater than about 25° C., greater than about 30° C., greater than about 40° C., greater than about 45° C., greater than about 50° C., greater than about 55° C., and/or greater than about 60° C.
12. The composition of embodiment 11, wherein the at least one core and/or at least two shells are responsive to at least one of: pH, temperature, time, light, water, living organisms, enzymes, bile salt, salt concentration, and/or mechanical forces.
13. The composition of embodiment 12, wherein at least one shell is responsive to a pH less than about 4.5.
14. The composition of embodiment 12, wherein the at least one core and the at least one shell is responsive to a pH greater than about 6.8.
15. The composition of embodiment 12, wherein the at least one core and the at least one shell responsive to a pH greater than about 6.8 are completely encapsulated in at least one shell responsive to a pH less than about 4.5.
16. The composition of embodiment 12, wherein at least one lipid, at least one protein, at least one carbohydrate, and/or at least one acaloric phytonutrient impart responsiveness to the at least one core and/or at least two shells.
17. The composition of embodiment 12, wherein the at least one core comprises at least one lipid, at least one protein, at least one carbohydrate, and/or at least one acaloric phytonutrient.
18. The composition of embodiment 12, wherein the at least two shells comprise at least one lipid, at least one protein, at least one carbohydrate, and/or at least one acaloric phytonutrient.
19. The composition of embodiment 1, wherein the at least one lipid, at least one protein, at least one carbohydrate, and/or at least one acaloric phytonutrient together comprise 100% of the dry weight.
20. The composition of embodiment 19, wherein the at least one lipid, at least one protein, at least one carbohydrate, and/or at least one acaloric phytonutrient(s) are characterized as safe for consumption by one or more mammal(s).
21. The composition of embodiment 20, wherein the at least one lipid, at least one protein, at least one carbohydrate, and/or at least one acaloric phytonutrient(s) are characterized as safe for consumption by human(s).
22. The composition of embodiment 21, wherein the at least one lipid, at least one protein, at least one carbohydrate, and/or at least one acaloric phytonutrient(s) are characterized as edible.
23. The composition of embodiment 22, wherein the at least one lipid comprises at least one component selected from the group consisting of fatty acids, fatty amides, sterols, terpenes, and terpenoids.
24. The composition of embodiment 23, wherein the at least one lipid characterized as a fatty acid and/or fatty amide is unsaturated.
25. The composition of embodiment 24, wherein the at least one lipid characterized as unsaturated is further characterized as all-cis.
26. The composition of embodiment 25, wherein the at least one unsaturated fatty acid and/or fatty amide possesses a carbon chain length between 10 and 26.
27. The composition of embodiment 26, wherein the fatty amide is an amide of ammonia, ethylamine, and/or ethanolamine.
28. The composition of embodiment 27, wherein the at least one lipid characterized as a fatty acid and/or fatty amide is all-cis α-linolenic acid, γ-linolenic acid, anandamide, arachidonic acid, adrenic acid, calendic acid, clupanodonic acid, docosadienoic acid, docosapentaenoic acid, docosahexaenoic acid, eicosadienoic acid, eicosapentaenoic acid, eicosatrienoic acid, elaidic acid, erucic acid, gondoic acid, linoleamide, linoleic acid, linolenylamide, mead acid, N-docosahexaenoylethanolamine, N-docosatetraenoylethanolamine, N-oleoylethanolamine, N-palmitoylethanolamine, N-stearoylethanolamine, nervonic acid, nisinic acid, oleamide, oleic acid, osbond acid, palmitoleic acid, pinolenic acid, paullinic acid, rumenic acid, stearidonic acid, tetracosapentaenoic acid, and/or vaccenic acid.
29. The composition of embodiment 28, wherein the at least one lipid characterized as a fatty acid and/or fatty amide exists is esterified and/or amidated to a glycerol backbone as a monoglyceride, a diglyceride, and/or a triglyceride.
30. The composition of embodiment 28, wherein the at least one lipid characterized as a fatty acid and/or fatty amide exists is esterified, and/or amidated to ethylene glycol, propylene glycol, sucrose, maltose, xylitol, erythritol, sorbitol, and/or mannitol.
31. The composition of embodiment 23, wherein the at least one lipid characterized as a sterol is γ-oryzanol, abietane, abietic acid, brassicasterol, campestanol, campesterol, cholestanol, cholesterol, ergosterol, sitostanol, sitosterol, oleanolic acid, ursolic acid, betulinic acid, moronic acid, cafestol, limonene, hinokitiol, carvone, menthol, linalool, thujene, and/or stigmasterol.
32. The composition of embodiment 22, wherein the at least one protein is a nutritional protein, a structural protein, an enzyme, a polypeptide, an oligopeptide, or an amino acid.
33. The composition of embodiment 32, wherein the at least one protein is calcium caseinate, corn protein isolate, milk protein concentrate, oat protein isolate, pea protein isolate, sodium caseinate, soy protein isolate, wheat protein isolate, and/or whey protein isolate.
34. The composition of embodiment 32, wherein the at least one protein is avenin, collagen, fibroin, gelatin, glutanin, kefirin, and/or zein.
35. The composition of embodiment 32, wherein the at least one protein is an amylase, a cellulase, a lipase, and/or a protease.
36. The composition of embodiment 32, wherein the at least one protein is amylin, cholecystokinin, GIP, GLP-1, GLP-2, oxyntomodulin, peptide YY, and/or somatostatin.
37. The composition of embodiment 32, wherein the at least one protein is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine.
38. The composition of embodiments 33-37, wherein the at least one protein is characterized as unmodified, denatured, hydrolyzed, and/or complexed.
39. The composition of embodiment 22, wherein the at least one carbohydrate is a monosaccharide, a disaccharide, and/or a polysaccharide.
40. The composition of embodiment 39, wherein the at least one carbohydrate is extracted from natural sources.
41. The composition of embodiment 39, wherein the at least one carbohydrate is characterized as fully synthetic.
42. The composition of embodiment 39, wherein the at least one carbohydrate is characterized as semi-synthetic.
43. The composition of embodiment 40, wherein the at least one carbohydrate extracted from natural sources is chemically modified.
44. The composition of embodiment 39, wherein the at least one carbohydrate is a nutrient.
45. The composition of embodiment 44, wherein the at least one carbohydrate is digestible.
46. The composition of embodiment 45, wherein the at least one carbohydrate is a source of energy.
47. The composition of embodiment 46, wherein the at least one carbohydrate is D-glucose, D-ribose, D-arabinose, D-allose, D-galactose, D-fructose, D-psicose, D-sucrose, D-tagatose, D-isomaltulose, D-lactose, D-maltose, D-trehalose, D-inositol.
48. The composition of embodiment 44, wherein the at least one carbohydrate is a source of dietary fiber.
49. The composition of embodiment 48, wherein the at least one carbohydrate is amylopectin, dextran, pectin, amylose, inulin, locust bean gum, maltodextrin, xanthan gum, gum arabic, karaya gum, ghatti gum, guar gum, sodium carboxymethylcellulose, sodium alginate, sodium hyaluronate, calcium alginate, agarose, chitosan, chitin, carrageenan, chondroitin sulfate, hydroxypropyl methylcellulose, methyl cellulose, modified cellulose gum, ethyl cellulose, hydroxyethylcellulose, corn starch, cellulose triacetate, cellulose acetate butyrate, cellulose, cellulose acetate propionate, cellulose acetate succinate, cellulose acetate phthalate, and/or hydroxypropyl methylcellulose acetate succinate.
50. The composition of embodiment 22, wherein the at least one acaloric phytonutrient is a flavonoid, an iridoid, and/or an alkylresorcinol.
51. The composition of embodiment 50, wherein the at least one acaloric phytonutrient is quercetin, kaempferol, myricetin, fisetin, rutin, isorhamnetin, naringenin, silybin, eriodictyol, apigenin, chrysin, delphinidin, betanin, cyanidin chloride, neohesperidin, epigallocatechin, diosmetin, baicalein, genistein, oleuropein, amarogentin, genipin, aucubin, catalpol, olivetol, cannabidiol, tetrahydrocannabinol, daidzein, pelargonidin, tangeritin, luteolin, wogonin, epicatechin, catechin, theaflavin, resveratrol, and/or hydroxytyrosol.
52. The composition of embodiment 13, wherein the at least one shell response to a pH less than 4.5 is characterized as buoyant, mucoadhesive, and/or retentive.
53. The composition of embodiment 1, wherein at least one shell component mitigates chemical and/or physical degradation of one or more lipid(s), protein(s), carbohydrate(s), and/or acaloric phytonutrient(s).
54. The composition of embodiment 53, wherein chemical degradation is characterized as a loss of greater than about 40% of the number and/or mass of one or more lipid(s), protein(s), carbohydrate(s), and/or acaloric phytonutrient(s) comprising one or more satiety modulating composition(s) upon a period of storage of at least 1 week, at least 1 month, at least 1 year, and/or at least 10 years at a temperature of at least 20° C., at least 40° C., and/or at least 80° C. in dry conditions.
55. The composition of embodiment 53, wherein chemical degradation is characterized as a loss of greater than about 40% of the number and/or mass of one or more lipid(s), protein(s), carbohydrate(s), and/or acaloric phytonutrient(s) comprising one or more satiety modulating composition(s) upon a period of storage of at least 1 week, at least 1 month, at least 1 year, and/or at least 10 years at a temperature of at least 20° C., at least 40° C., and/or at least 80° C. in high humidity conditions.
56. The composition of embodiment 53, wherein chemical degradation is characterized as a loss of greater than about 40% of the number and/or mass of one or more lipid(s), protein(s), carbohydrate(s), and/or acaloric phytonutrient(s) comprising one or more satiety modulating composition(s) upon a period of storage of at least 1 week, at least 1 month, at least 1 year, and/or at least 10 years at a temperature of at least 20° C., at least 40° C., and/or at least 80° C. in solution.
57. The composition of embodiment 1, wherein the water activity exhibited by the particle preparation is <about 0.4, <about 0.3, <about 0.2, and/or <about 0.1.
58. The composition of embodiment 1, wherein the total mass of one or more lipid(s), protein(s), carbohydrate(s), and acaloric phytonutrient(s) is at least about 0.1 g, at least about 0.5 g, at least about 1 g, at least about 5 g, at least about 10 g, at least about 50 g, and/or at least about 100 g.
59. The composition of embodiment 58, wherein the total mass of one or more lipid(s), protein(s), carbohydrate(s), and acaloric phytonutrient(s) is less than about 500 g.
60. The composition of embodiment 1, wherein the caloric content of one or more lipid(s), protein(s), and carbohydrate(s) is at least about 0 kcal, at least about 5 kcal, at least about 20 kcal, at least about 50 kcal, at least about 100 kcal, and/or at least about 250 kcal.
61. The composition of embodiment 60, wherein the caloric content of one or more lipid(s), protein(s), and carbohydrate(s) is less than about 500 kcal.
62. The composition of embodiment 1, wherein the particle preparation is amenable to incorporation into one or more food and/or beverage product(s).
63. The composition of embodiment 62, wherein the particle preparation is stable to size reduction techniques.
64. The composition of embodiment 63, wherein size reduction techniques comprise planetary milling, ball milling, burr milling, roller milling, media milling, impact milling, jet milling, high-pressure homogenization, cryo milling, hammer milling, conical milling, hand screening, or granulation/extrusion, extrusion, spray drying, lyophilization/milling, fluid bed agglomeration, spray congealing, high-shear granulation, tableting, pouring, roller compaction, crosslinking, prilling, spinning disc atomization, and/or combinations thereof.
65. The composition of embodiment 62, wherein the particle preparation is stable to homogenization techniques.
66. The composition of embodiment 62, wherein homogenization techniques comprise overhead stirrer, manual stirring, stir bar, high pressure homogenization, low pressure homogenization, sonication, ultrasonication, vortexing, or combinations thereof.
67. The composition of embodiment 62, wherein food and/or beverage product(s) comprise agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, tablet, yogurt, liquid supplement formulation, beer, seltzer, kefir, coffee, juice, liquid pharmaceutical formulation, milk, soda, sports drink, tea, water, liquor, and/or wine.
68. A non-therapeutic method of controlling satiety comprising:
a step of dispersing one or more nutraceutical particle preparations comprising multi-layer core-shell spatial arrangement(s) of about 40% to about 75%, on a dry weight basis, of one or more lipid(s), about 10% to about 30%, on a dry weight basis, of one or more protein(s), about 10% to about 30%, on a dry weight basis, of one or more carbohydrate(s), and about 5% to about 15%, on a dry weight basis, of one or more acaloric phytonutrient(s) characterized by release of at least 90% of the at least one lipid, at least one protein, at least one carbohydrate, and/or at least one acaloric phytonutrient in both upper gastrointestinal and lower gastrointestinal regions of one or more mammal(s) within a food and/or beverage matrix; and
a step of administering an effective dose of said dispersion to one or more mammal(s).
69. The method of embodiment 68, wherein a food and/or beverage matrix is characterized as a food and/or beverage ingredient, an unfinished food and/or beverage product, a finished food and/or beverage product, and/or a food and/or beverage supplement.
70. The method of embodiment 69, wherein <about 10% of one or more lipid(s), protein(s), carbohydrate(s), and/or acaloric phytonutrient(s) is observed in one or more food and/or beverage matrices.
71. The method of embodiment 70, wherein >about 90% of one or more lipid(s), protein(s), carbohydrate(s), and/or acaloric phytonutrient(s) is observed in the gastrointestinal tract of one or more mammal(s).
72. The method of embodiment 71, wherein >about 40% of one or more lipid(s), protein(s), carbohydrate(s), and/or acaloric phytonutrient(s) is observed in the upper gastrointestinal tract of one or more mammal(s).
73. The method of embodiment 71, wherein >about 50% of one or more lipid(s), protein(s), carbohydrate(s), and/or acaloric phytonutrient(s) is observed in the lower gastrointestinal tract of one or more mammal(s).
74. The method of embodiment 73, wherein one or more shell component(s) releases in the upper gastrointestinal tract of one or more mammal(s).
75. The method of embodiment 73, wherein one or more core component(s) releases in the lower gastrointestinal tract of one or more mammal(s).
76. The method of embodiment 69, wherein one or more particle preparation(s) is further characterized as a pill, a crystal, an emulsion, an oil, a dispersion, a tablet, a gel-cap, a solution, a gel, a powder, a sachet, a bar, a granule, a particle preparation, a core-shell preparation, a capsule, a jelly, a suspension, an elixir, a syrup, a food, and/or a beverage.
77. The method of embodiment 76, wherein one or more particle preparation(s) are administered orally, rectally, percutaneously, subcutaneously, and/or intravenously.
78. The method of embodiment 68, wherein an effective quantity of one or more lipid(s), protein(s), carbohydrate(s), and acaloric phytonutrient(s) is at least about 0.1 g, at least about 0.5 g, at least about 1 g, at least about 5 g, at least about 10 g, at least about 50 g, and/or at least about 100 g.
79. The method of embodiment 78, wherein the effective quantity of one or more lipid(s), protein(s), carbohydrate(s), and acaloric phytonutrient(s) is less than about 500 g.
80. The method of embodiment 68, wherein the effective quantity of one or more lipid(s), protein(s), and carbohydrate(s) is at least about 0 kcal, at least about 5 kcal, at least about 20 kcal, at least about 50 kcal, at least about 100 kcal, and/or at least about 250 kcal.
81. The method of embodiment 80, wherein the caloric content of one or more lipid(s), protein(s), and carbohydrate(s) is less than about 500 kcal.
82. The method of embodiment 68, wherein a step of administering a satiety modulating composition is measured as effective by subjective, self-reported grading by one or more mammal(s), relative to post-absorptive levels.
83. The method of embodiment 68, wherein a step of administering a satiety modulating composition is measured as effective by quantifying a change of at least 20% in at least one of leptin, GLP-1, GLP-2, motilin, gastrin, insulin, ghrelin, peptide yy, cholecystokinin, GIP, serotonin, histamine, oleoylethanolamide, palmitoylethanolamide, anandamide, and/or linolenoylethanolamide, their prehormones, their isoforms, their degradation products, and/or their full length and/or spliced transcripts in the serum of one or more mammal(s), relative to post-absorptive levels.
84. The method of embodiment 83, wherein satiety is controlled for at least about 6, about 12, about 16, and/or about 24 hours.

### VIII. EXEMPLIFICATION

The following examples are intended to illustrate but not limit the disclosed embodiments. The following examples are useful to confirm aspects of the disclosure described above and to exemplify certain embodiments of the disclosure.

These non-limiting examples demonstrate particular features and advantages of provided technologies—e.g., of provided satiety modulating composition(s).

Among other things, provided satiety modulating composition(s) may be characterized by significant improvements, including, for example, (i) non-therapeutic satiety modulating composition(s), (ii) improved changes in circulating levels of several satiety hormone(s), (iii) satiety modulating composition(s) are primarily comprised of edible satiety modulator(s), (iv) satiety modulator(s) not known in the prior art, (v) novel satiety modulator(s) enhancing the efficacy of known satiety modulator(s), (vi) core-shell structure(s) for targeting of multiple gastrointestinal region(s), (vii) improved delivery of payloads (e.g., macronutrients, ratios of proteins to fats to carbohydrates) to specific sites in the gastrointestinal tract (e.g., ileum), (viii) improved delivery of payloads (e.g., macronutrients, ratios of proteins, fats, carbohydrates) to specific cells in the gastrointestinal tract (e.g., L cells), (ix) improved delivery of a specific ratio of macronutrient payloads (e.g., ratios of proteins to fats to carbohydrates) that modulates satiety, (x) improved delivery of a specific concentration of macronutrient payloads that modulates satiety, (xi) spatiotemporal control over the delivery of a specific ratio and/or a specific concentration of macronutrient payloads that modulates satiety, (xii) compatibility with and/or in food and/or food products, with and/or in beverages and/or beverage products, with and/or in supplements, with and/or in dry powders, (xiii) improved shelf-life and resistance to degradation at/in decreased temperatures (e.g., −80° C., −20° C., and/or 4° C.), ambient temperatures (e.g., 10° C., 15° C., and/or 20° C.), elevated temperatures (e.g., 22° C., 25° C., 30° C., 35° C., and/or 40° C.), the presence of high relative humidity (e.g., up to 100%), the presence of moisture, the presence of oxygen, the presence of light, the presence of heat, the presence of surfactants, in liquids (acidic liquids (pH values between 1-6), neutral liquids (pH values between 6-8), basic liquids (pH values between 8-12)), or a combination thereof, (xiv) prolonged residence time or transit time in the gastrointestinal tract or gastrointestinal tract compartments, (xv) controlled release or sustained release of payload components in the gastrointestinal tract, (xvi) controlled spatial distribution of payloads in and/or on the gastrointestinal tract, (xvii) controlled concentration of payloads in the gastrointestinal tract (e.g., in the stomach, in the intestines, at the epithelial surface, in the mucus, etc.), (xviii) improved shelf-life in food or beverage matrices, protein bars, dry powders, milk powders, whey powders, yogurt, drinkable yogurt, (ixx) improved payload resistance to losses during manufacturing processes: pasteurization, shear mixing, elevated pressurized processes, elevated temperature processes, or combinations thereof, (xx) tunable properties including size, coating thickness, morphology, geometry, loading, dose, interactions with the surrounding environment, release conditions, or combinations thereof, (xxi) maintenance and preservation of composition morphology (e.g., particle geometry) when exposed to typically degrading conditions, (xxii) changes in circulating satiety hormone concentrations by at least 20%, (xxiii) satiety is controlled for at least 4 hours, at least 6 hours, at least 12 hours, and/or at least 24 hours.

### A. Example 1: Caloric Content and Satiety Modulators in Exemplary Satiety Modulating Compositions

In one exemplary, non-limiting, embodiment, one or more satiety modulating composition(s) is comprised of at least one satiety modulator arranged in a core-shell particle preparation. In the example provided herein, one or more satiety modulating composition(s) is or may be characterized by the total mass of one satiety modulator. In the example provided herein, one or more satiety modulating composition(s) is or may be characterized by the caloric content of one satiety modulator. Table 1, in three non-limiting instances, illustrates satiety modulating composition(s) characterized by at least one of mass and/or caloric content.

TABLE 1
Exemplary Satiety Modulating Compositions

| | | Example | | Example | | Example | |
|---|---|---|---|---|---|---|---|
| | | Composition 1 | | Composition 2 | | Composition 3 | |
| Lipid(s) | Name | Oleic acid | | Oleic acid + | | Docosahexaenoic | |
| | | | | Sitosterol | | Acid | |
| | Mass | 705 | mg/g | 617 mg/g + | | 760 | mg/g |
| | | | | 95 mg/g | | | |
| | Calories | 6.3 | kcal/g | 5.5 | kcal/g | 6.8 | kcal/g |
| Carbohydrate(s) | Name | Ethyl cellulose | | Glucose | | Inulin | |
| | Mass | 150 | mg/g | 217 | mg/g | 190 | mg/g |
| | Calories | 0 | kcal/g | 0.9 | kcal/g | 0 | kcal/g |
| Acaloric | Name | Cyanidin Chloride | | Genipin | | Wogonin | |
| Phytonutrient(s) | Mass | 145 | mg/g | 71 | mg/g | 50 | mg/g |
| | Calories | 0 | kcal/g | 0 | kcal/g | 0 | kcal/g |

### B. Example 2: Encapsulation of Carbohydrates

In certain embodiments, one or more satiety modulator(s) characterized as a carbohydrate is or are encapsulated in one or more core-shell preparations. In one exemplary embodiment, one or more carbohydrate(s) is or are encapsulated in a core-shell particle preparation disclosed herein.

For example, in one non-limiting embodiment a satiety modulating composition comprised of a carbohydrate further characterized as a monosaccharide is embedded within a carbohydrate further characterized as a polysaccharide, demonstrating exemplary encapsulation of a carbohydrate. For example, in one non-limiting embodiment, a satiety modulating composition comprised of a carbohydrate further characterized as a polysaccharide is embedded within a carbohydrate further characterized as a polysaccharide, demonstrating exemplary encapsulation of a carbohydrate. For example, in one non-limiting embodiment, a satiety modulating composition comprised of an encapsulated carbohydrate is encapsulated by a lipid (e.g., a core-shell particle preparation). For example, in a non-limiting embodiment, a satiety modulating composition comprising an encapsulated carbohydrate is encapsulated by a protein (e.g., a core-shell particle preparation).

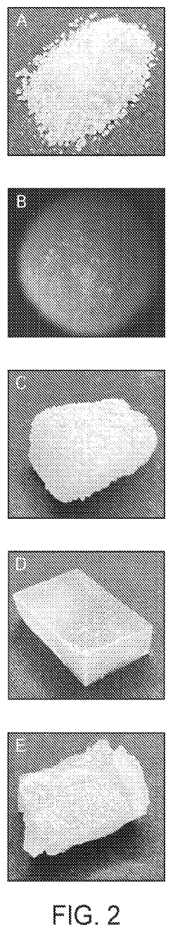

FIG. 2 illustrates a comparison between gross morphologies of unencapsulated (FIG. 2A) and encapsulated (FIGS. 2B-E) carbohydrates. For example, in FIG. 2B, sucrose is dispersed within a wet amylose matrix, the slurry sprayed into cool air to generate particles of controlled diameter. The encapsulation of sucrose within amylose is calculated to be 92%, on a dry weight basis. For example, in FIG. 2B, particles comprising encapsulated sucrose (92% loading, 1 mm diameter) are further coated (e.g., spray pan coating) with a 90% (v/v) solution of Zein with a colorant (e.g., excipient component). Brightfield micrographs reveal increased surface roughness and a red coloring, indicative of successful coating. For example, in FIG. 2C, 14 g of glucose is added to 6 mL of a stirring 5% (w/v) pectin solution held at 110° C. and the resulting mixture is stirred for 3 minutes, or until all glucose is dissolved. 1.0 mL of 1 M citric acid in distilled water is added to the stirring mixture, which is further mixed for 15 seconds before pouring into molds coated with loose glucose crystals and setting for 1 hour at 20° C. For example, in FIG. 2D, 100 mg of inulin (from dahlia tubers) is dissolved in 5 mL of distilled water and warmed to 60° C. while stirring. 5 mL of a 5% (w/v) solution of agarose at 130° C. is added to the stirring mixture, which is further mixed for 15 seconds before pouring into molds and setting for 1 hour at 20° C. For example, in FIG. 2E, 10 mL of a stirring 20% (w/v) suspension of calcium caseinate is added to 10 mL of a stirring 60° C. mixture of 5% (w/v) solution of sodium alginate and 2% (w/v) inulin (from dahlia tuber). The resulting mixture is allowed to stir for 15 seconds before pouring into molds and setting for 1 hour at 20° C. Unlike the free-flowing powder depicted in FIG. 2A, the formulations generated using the methods of manufacture outlined for FIG. 2C-E are cohesive particle preparation(s) indicating successful encapsulation.

### C. Example 3: Encapsulation of Proteins

In certain embodiments, one or more satiety modulator(s) characterized as a protein is or are encapsulated in one or more core-shell particle preparations.

For example, in one non-limiting embodiment, a satiety modulating composition comprised of a protein further characterized as an amino acid is embedded within a carbohydrate further characterized as a polysaccharide, demonstrating exemplary encapsulation of a protein. For example, in one non-limiting embodiment, a satiety modulating composition comprised of a protein further characterized as a nutritional protein is embedded within a carbohydrate further characterized as a polysaccharide, demonstrating exemplary encapsulation of a protein. For example, in one non-limiting embodiment, a satiety modulating composition comprised of an encapsulated protein is encapsulated by a lipid (e.g., a core-shell preparation). For example, in one non-limiting embodiment, a satiety modulating composition comprised of an encapsulated protein is encapsulated by a protein (e.g., a core-shell preparation).

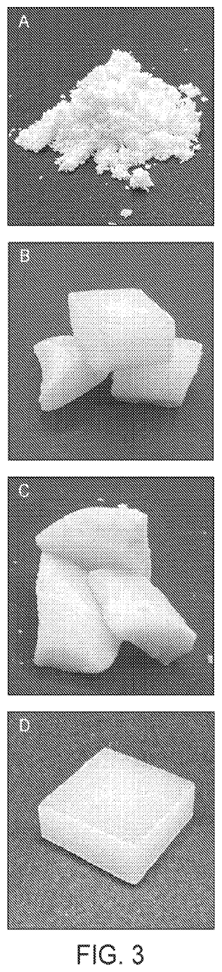

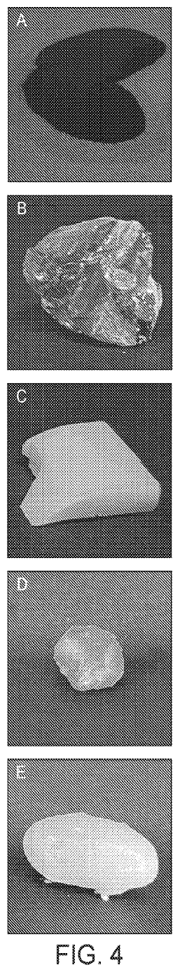

FIG. 3 illustrates a comparison between gross morphologies of unencapsulated (FIG. 3A, whey protein isolate) and encapsulated (FIGS. 3B-D, encapsulated) protein. For example, in FIG. 3B, whey protein isolate powder is dispersed at 10000 rpm using a high-shear homogenizer within molten beeswax and the resulting dispersion is poured into a mold to set for 1 hour at 20° C. For example, in FIG. 3C, whey protein isolate powder is dispersed at 10000 rpm using a high-shear homogenizer within molten hydrogenated soy oil and the resulting dispersion is poured into a mold to set for 1 hour at 20° C. For example, in FIG. 3D, 5 mL of 5% (w/v) agarose solution at 130° C. is mixed with 5 mL of a solution comprising 20% (w/v) whey protein isolate and 2% (w/v) chitosan) at pH 5 and 60° C. The resulting mixture is allowed to stir for 15 seconds before pouring into molds and setting for 1 hour at 20° C. Unlike the free-flowing powder depicted in FIG. 3A, the formulations generated using the methods of manufacture outlined for FIG. 3B-D are cohesive particle preparation(s) indicating successful encapsulation.

### D. Example 4: Encapsulation of Lipids

In certain embodiments, one or more satiety modulator(s) characterized as a lipid is or are encapsulated in one or more core-shell preparations.

For example, in one non-limiting embodiment, a satiety modulating composition comprised of a lipid further characterized as a fatty acid is embedded within a carbohydrate further characterized as a polysaccharide, demonstrating exemplary encapsulation of a lipid. For example, in one non-limiting embodiment, a satiety modulating composition comprised of a lipid further characterized as a sterol is embedded within a carbohydrate further characterized as a polysaccharide, demonstrating exemplary encapsulation of a lipid. For example, in one non-limiting embodiment, a satiety modulating composition comprised of an encapsulated lipid is encapsulated by a lipid (e.g., a core-shell preparation). For example, in one non-limiting embodiment, a satiety modulating composition comprised of an encapsulated lipid is encapsulated by a protein (e.g., a core-shell preparation). In one non-limiting embodiment, a core-shell preparation is further characterized as a particle preparation.

FIG. 4 illustrates a comparison between gross morphologies of unencapsulated (FIG. 4A, oleic acid) and encapsulated (FIGS. 4B-E, encapsulated) lipid. For example, in FIG. 4B, 8 mL of oleic acid is heated, while stirring, to 130° C., followed by the addition of 2.0 g of ethyl cellulose (100 cP). The mixture is kept stirring at 130° C. for 10 minutes, or until all solids are dissolved, and the resulting clear solution is then poured into an aluminum pan to set at 20° C. for 1 hour. For example, in FIG. 4C, 8 mL of oleic acid is heated, while stirring, to 90° C., followed by the addition of 2.0 g of beeswax. The mixture is kept stirring at 90° C. for 10 minutes, or until all solids are dissolved, and the resulting clear solution is poured into an aluminum pan to set at 20° C. for 1 hour. For example, in FIG. 4D, 6.5 mL of oleic acid is heated, while stirring, to 130° C., followed by the addition of 1.0 g of carnauba wax and 1.5 g of ethyl cellulose. The mixture is kept stirring at 130° C. for 10 minutes, or until all solids are dissolved, and 1.0 g of whey protein isolate is added. After 15 seconds of stirring, the suspension is poured into an aluminum pan and allowed to set at 20° C. for 1 hour. The resulting waxy solid is then immersed and wrapped in an interfacial thin film comprised of chitosan-polyphosphate and allowed to dry at 50° C. for 6 hours. For example, in FIG. 4E, core component(s) prepared as described in FIG. 4D are instead coated by dipping into a 15% (w/v) solution of cellulose acetate phthalate in acetone and drying under air at 40° C. Unlike the loose oil depicted in FIG. 4B, the formulations generated using the methods of manufacture outlined for FIG. 4B-E are solid, cohesive particle preparation(s) indicating successful encapsulation.

### E. Example 5: Release of Carbohydrates from One or More Satiety Modulating Composition(s)

In certain embodiments, one or more satiety modulator(s) characterized as a carbohydrate is or are encapsulated in one or more core-shell preparations. In certain embodiments, the encapsulation of one or more carbohydrate(s) in one or more core-shell preparation(s) is further characterized by the release of encapsulated carbohydrate(s) in one or more release environment(s). In some instances, the release profile(s), as provided herein, of carbohydrate(s) encapsulated in one or more core-shell preparation(s) illustrate controlled release of carbohydrate(s).

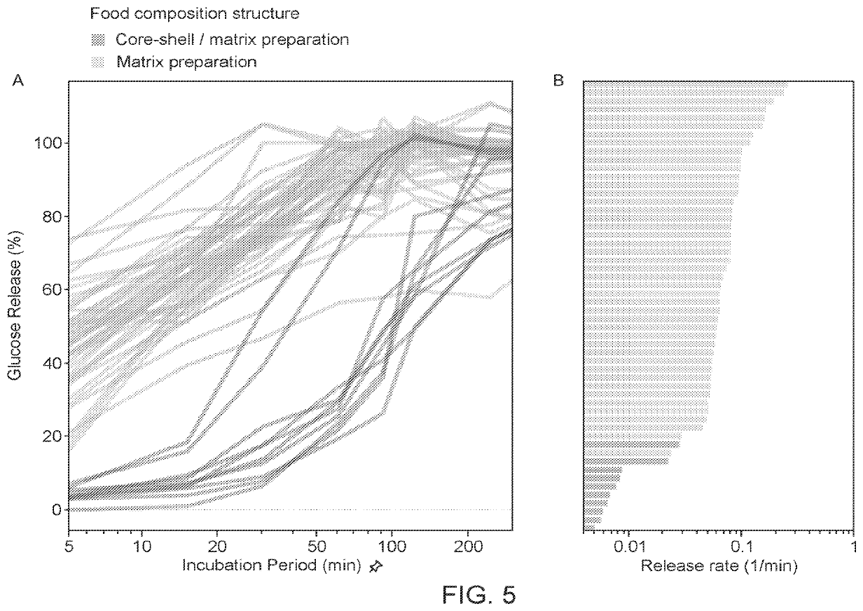

In some non-limiting embodiments, one or more satiety modulating composition(s) comprising carbohydrate(s) further characterized as core-shell preparation(s) provide a means of controlling carbohydrate release. In certain embodiments, the selection of one or more core component(s) and/or shell component(s) and their relative concentration(s) provide a means of controlling release of carbohydrate(s). In one non-limiting embodiment (FIG. 5), the average release profiles (n=3) of an exemplary carbohydrate encapsulated within satiety modulating composition(s) comprising distinct core component(s) and/or shell component(s) within a release environment comprising phosphate buffered saline, pH 7.4 at 37° C. are depicted. In this example, exemplary release profile(s) are provided by satiety modulating composition(s) further characterized as core-shell preparation(s). In some instances, the release profile(s) provided by one or more satiety modulating composition(s) changes in different release environment(s). In some instances, the release profile(s) provided by one or more satiety modulating composition(s) remains constant in different release environment(s). In certain preferred embodiments, the release profile(s) provided by one or more satiety modulating composition(s) illustrated in FIG. 5 enable the selection of satiety modulating composition(s) conferring desirable release profile(s) upon one or more carbohydrates, for example, to reduce meal frequency in one or more mammal(s).

In some non-limiting embodiments, one or more satiety modulating composition(s) providing for 80% release of encapsulated carbohydrate within 30 minutes is or may be advantageous. In some non-limiting embodiments, one or more satiety modulating composition(s) providing for 80% release of encapsulated carbohydrate within 100 minutes is or may be advantageous. In some non-limiting embodiments, one or more satiety modulating composition(s) providing for 80% release of encapsulated carbohydrate within 240 minutes is or may be advantageous.

As shown in FIG. 5, the time required to reach 80% (w/w) release of encapsulated carbohydrate(s) can vary from ~7 minutes to >280 min depending on the concentration(s) and/or identity of one or more core component(s) and/or shell component(s) comprising one or more satiety modulating composition(s). A complete list of exemplary satiety modulating composition(s), their associated core component(s) and/or shell component(s), their respective concentration(s), and release rates are provided in Appendix 1. In some cases, the exemplary release profiles provided in FIG. 5 aid in the selection of desirable satiety modulating composition structure (e.g., core-shell preparation, particle preparation). In some cases, the exemplary release profiles provided in FIG. 5 aid in the selection and relative concentration(s) of one or more core component(s) and/or shell component(s).

As shown in FIG. 5, the release rate of glucose from one or more exemplary satiety modulating composition(s) varies substantially by satiety modulating composition structure (e.g., core-shell preparation, particle preparation). Core-shell preparations generally offer slower release kinetics (<0.03 min⁻¹) relative to uncoated core components (>0.03 min⁻¹).

### F. Example 6: Release of Proteins from One or More Satiety Modulating Composition(s)

In certain embodiments, one or more satiety modulator(s) characterized as a protein is or are encapsulated in one or more core-shell preparations. In certain embodiments, the encapsulation of one or more protein(s) in one or more core-shell preparation(s) is further characterized by the release of encapsulated protein(s) in one or more release environment(s). In some instances, the release profile(s), as provided herein, of protein(s) encapsulated in one or more core-shell preparation(s) illustrate controlled release of protein(s).

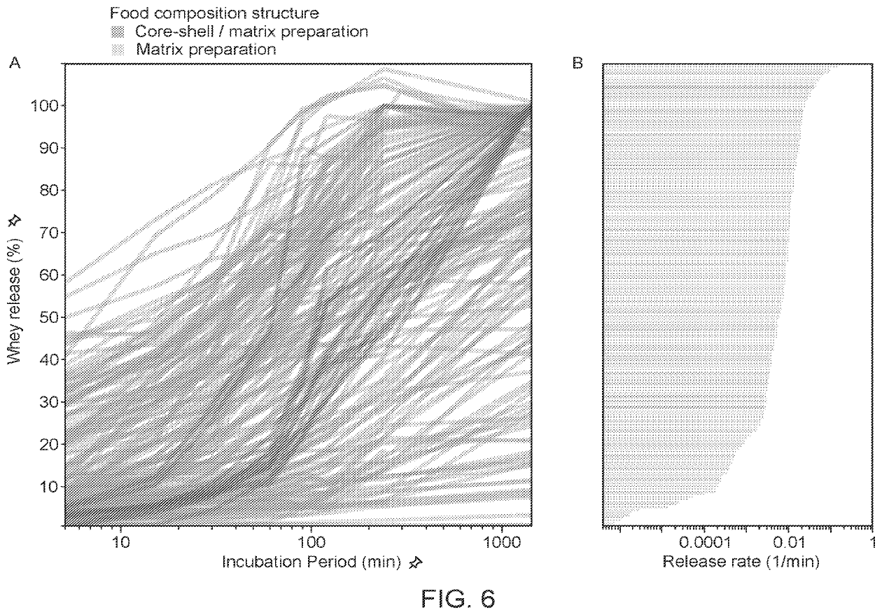

In some non-limiting embodiments, one or more satiety modulating composition(s) comprising protein(s) further characterized as core-shell preparation(s) provides a means of controlling protein release. In certain embodiments, the selection of one or more core component(s) and/or shell component(s) and their relative concentration(s) provide a means of controlling release of protein(s). In one non-limiting embodiment (FIG. 6), the average release profiles (n=3) of an exemplary protein encapsulated within satiety modulating composition(s) comprising distinct core component(s) and/or shell component(s) within a release environment comprising phosphate buffered saline, pH 7.4 at 37° C. are depicted. In this example, exemplary release profile(s) are provided by satiety modulating composition(s) further characterized as core-shell preparation(s). In some instances, the release profile(s) provided by one or more satiety modulating composition(s) changes in different release environment(s). In some instances, the release profile(s) provided by one or more satiety modulating composition(s) remains constant in different release environment(s). In certain preferred embodiments, the release profile(s) provided by one or more satiety modulating composition(s) illustrated in FIG. 6 enable the selection of satiety modulating composition(s) conferring desirable release profile(s) upon one or more proteins, for example, to reduce meal frequency in one or more mammal(s).

In some non-limiting embodiments, one or more satiety modulating composition(s) providing for 80% release of encapsulated protein within 60 minutes is or may be advantageous. In some non-limiting embodiments, one or more satiety modulating composition(s) providing for 80% release of encapsulated protein within 200 minutes is or may be advantageous. In some non-limiting embodiments, one or more satiety modulating composition(s) providing for 80% release of encapsulated protein within 1000 minutes is or may be advantageous.

As shown in FIG. 6, the time required to reach 80% (w/w) release of encapsulated protein(s) can vary from ~30 minutes to >1000 min depending on the concentration(s) and/or identity of one or more core component(s) and/or shell component(s) comprising one or more satiety modulating composition(s). A complete list of exemplary satiety modulating composition(s), their associated core component(s) and shell component(s) and their respective concentration(s), and release rates are provided in Appendix 1. In some cases, the exemplary release profiles provided in FIG. 6 aid in the selection of desirable satiety modulating composition structure (e.g., core-shell preparation, particle preparation). In some cases, the exemplary release profiles provided in FIG. 6 aid in the selection and relative concentration(s) of one or more core component(s) and/or shell component(s).

As shown in FIG. 6, the release rate of protein from one or more exemplary satiety modulating composition(s) remains consistent regardless of satiety modulating composition structure (e.g., core-shell preparation, particle preparation). Core-shell preparations wherein one or more core component(s) further comprise encapsulated satiety modulator(s) offer comparable release kinetics (0.002-0.02 min⁻¹) relative to the majority of uncoated core preparation(s) (0.001-0.03 min⁻¹).

### G. Example 7: Release of Lipids from One or More Satiety Modulating Composition(s)

In certain embodiments, one or more satiety modulator(s) characterized as a lipid is or are encapsulated in one or more core-shell preparations. In certain embodiments, the encapsulation of one or more lipid(s) in one or more core-shell preparation(s) is further characterized by the release of encapsulated lipid(s) in one or more release environment(s). In some instances, the release profile(s), as provided herein, of lipid(s) encapsulated in one or more core-shell preparation(s) illustrate controlled release of lipid(s).

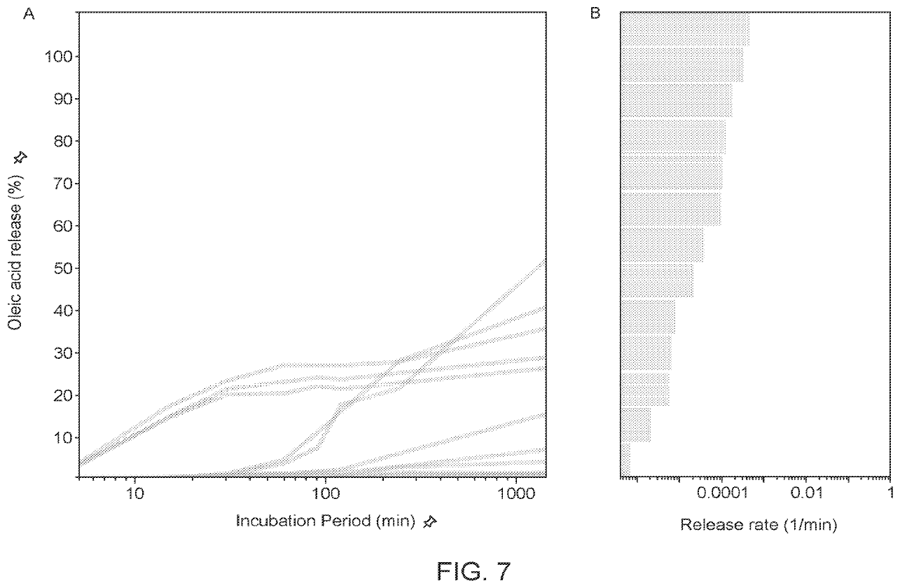

In some non-limiting embodiments, one or more satiety modulating composition(s) comprising lipid(s) further characterized as core-shell preparation(s) provides a means of controlling lipid release. In certain embodiments, the selection of one or more core component(s) and/or shell component(s) and their relative concentration(s) provide a means of controlling release of lipid(s). In one non-limiting embodiment (FIG. 7), the average release profiles (n=3) of an exemplary lipid encapsulated within satiety modulating composition(s) comprising distinct core component(s) and/or shell component(s) within a release environment comprising phosphate buffered saline, pH 7.4 at 37° C. are depicted. In this example, exemplary release profile(s) are provided by satiety modulating composition(s) further characterized as core-shell preparation(s). In some instances, the release profile(s) provided by one or more satiety modulating composition(s) changes in different release environment(s). In some instances, the release profile(s) provided by one or more satiety modulating composition(s) remains constant in different release environment(s). In certain preferred embodiments, the release profile(s) provided by one or more satiety modulating composition(s) illustrated in FIG. 7 enable the selection of satiety modulating composition(s) conferring desirable release profile(s) upon one or more lipids, for example, to reduce meal frequency in one or more mammal(s).

In some non-limiting embodiments, one or more satiety modulating composition(s) providing for 20% release of encapsulated lipid within 20 minutes is or may be advantageous. In some non-limiting embodiments, one or more satiety modulating composition(s) providing for 20% release of encapsulated lipid within 200 minutes is or may be advantageous. In some non-limiting embodiments, one or more satiety modulating composition(s) providing for 20% release of encapsulated lipid within 1000 minutes is or may be advantageous.

As shown in FIG. 7, the time required to reach 20% (w/w) release of encapsulated lipid(s) can vary from ~30 minutes to >1000 min depending on the concentration(s) and/or identity of one or more core component(s) and/or shell component(s) comprising one or more satiety modulating composition(s). A complete list of exemplary satiety modulating composition(s), their associated core component(s) and shell component(s) and their respective concentration(s), and release rates are provided in Appendix 1. In some cases, the exemplary release profiles provided in FIG. 7 aid in the selection of desirable satiety modulating composition structure (e.g., core-shell preparation, particle preparation). In some cases, the exemplary release profiles provided in FIG. 7 aid in the selection and relative concentration(s) of one or more core component(s) and/or shell component(s).

As shown in FIG. 7, the release rate of lipid from one or more exemplary satiety modulating composition(s) characterized as core-shell preparation(s) varies depending on selected core component(s). Exemplary core component(s) comprising ethyl cellulose confer a release rate of ~0.001 min⁻¹, while core component(s) comprising sitosterol confer a release rate of ~0.000001 min⁻¹.

### H. Example 8: Encapsulation and Release of Satiety Modulator(s)

In certain embodiments, one or more satiety modulator(s) characterized as a lipid is or are encapsulated in one or more core-shell preparations. In certain embodiments, the encapsulation of one or more lipid(s) in one or more core-shell preparation(s) is further characterized by the release of encapsulated lipid(s) in one or more release environment(s). In some instances, the release profile(s), as provided herein, of lipid(s) encapsulated in one or more core-shell preparation(s) illustrate controlled release of lipid(s). As illustrated in FIG. 4 and FIG. 7, an exemplary lipid encapsulated in provided satiety modulating composition(s) may be oleic acid. Additionally, in some embodiments, linoleic acid, docosahexaenoic acid, and/or eicosapentaenoic acid may be encapsulated and exhibit controlled release within and from provided satiety modulating composition(s), respectively.

In certain embodiments, one or more satiety modulator(s) characterized as a protein is or are encapsulated in one or more core-shell preparations. In certain embodiments, the encapsulation of one or more protein(s) in one or more core-shell preparation(s) is further characterized by the release of encapsulated protein(s) in one or more release environment(s). In some instances, the release profile(s), as provided herein, of protein(s) encapsulated in one or more core-shell preparation(s) illustrate controlled release of protein(s). As illustrated in FIG. 3 and FIG. 6, an exemplary protein encapsulated in provided satiety modulating composition(s) may be whey protein. Additionally, in some embodiments, gelatin, collagen, casein, oat protein isolate, soy protein isolate, and/or pea protein isolate may be encapsulated and exhibit controlled release within and from provided satiety modulating composition(s), respectively.

In certain embodiments, one or more satiety modulator(s) characterized as an acaloric phytonutrient is or are encapsulated in one or more core-shell preparations. In certain embodiments, the encapsulation of one or more acaloric phytonutrient(s) in one or more core-shell preparation(s) is further characterized by the release of encapsulated acaloric phytonutrient(s) in one or more release environment(s). In some instances, the release profile(s), as provided herein, of acaloric phytonutrient(s) encapsulated in one or more core-shell preparation(s) illustrate controlled release of acaloric phytonutrient(s).

For example, in one non-limiting embodiment, a satiety modulating composition comprised of an acaloric phytonutrient (e.g., a solute component) is embedded within a protein (e.g., a shell component), demonstrating exemplary encapsulation of an acaloric phytonutrient.

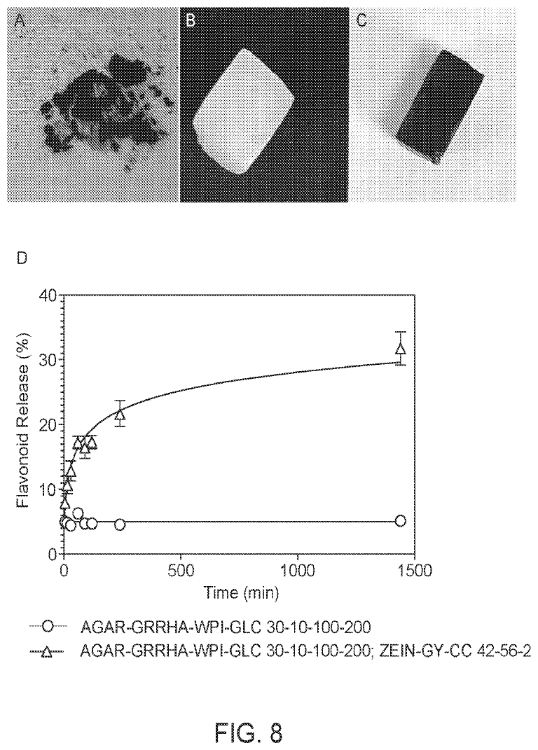

FIG. 8 illustrates a comparison between gross morphologies of unencapsulated (FIG. 8A, cyanidin chloride) and encapsulated (FIG. 8C, encapsulated) acaloric phytonutrient. For example, an ethanol solution (90% (w/v)) of zein is prepared with the addition of 1% (w/w) cyanidin chloride to form a purple solution, the purple solution then applied to a core component comprising agarose and glucose (FIG. 8B) via paint coating. Unlike the loose powder depicted in FIG. 8A, the formulations generated using the methods of manufacture outlined are solid, cohesive particle preparation(s) with a smooth coating, indicating successful encapsulation.

In one non-limiting example, encapsulated acaloric phytonutrient satiety modulating composition(s) demonstrate controlled release. For example, FIG. 8D illustrates a comparison in flavonoid release from a formulation containing encapsulated cyanidin chloride (3% (w/w) agarose, 1% (w/w) glycyrrhetinic acid, 10% (w/w) whey protein isolate, and 20% (w/w) glucose in the core, 42% (w/w) Zein, 56% (w/w) glycerol, and 2% (w/w) cyanidin chloride in the shell) (black triangles) and a formulation containing no cyanidin chloride (3% (w/w) agarose, 1% (w/w) glycyrrhetinic acid, 10% (w/w) whey protein isolate, and 20% (w/w) glucose in the core) (black circles). In this example, flavonoid releases from the exemplary satiety modulating composition(s), reaching 30% release by 24 hours in phosphate buffered saline, pH 7.4.

In certain embodiments, one or more satiety modulator(s) characterized as a carbohydrate is or are encapsulated in one or more core-shell preparations. In certain embodiments, the encapsulation of one or more carbohydrate(s) in one or more core-shell preparation(s) is further characterized by the release of encapsulated carbohydrate(s) in one or more release environment(s). In some instances, the release profile(s), as provided herein, of carbohydrate(s) encapsulated in one or more core-shell preparation(s) illustrate controlled release of carbohydrate(s). As illustrated in FIG. 2 and FIG. 5, an exemplary carbohydrate encapsulated in provided satiety modulating composition(s) may be glucose. Additionally, in some embodiments, sucrose, tagatose, psicose, and/or isomaltulose may be encapsulated and exhibit controlled release within and from provided satiety modulating composition(s), respectively.

For example, in one non-limiting embodiment, a satiety modulating composition comprised of inulin (e.g., a solute component) is embedded within a carbohydrate (e.g., a core component), demonstrating exemplary encapsulation of a carbohydrate.

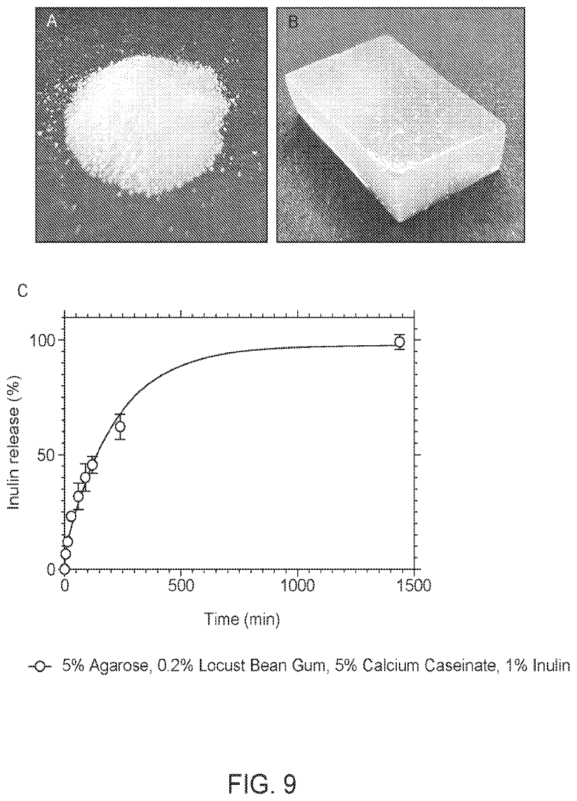

FIG. 9 illustrates a comparison between gross morphologies of unencapsulated (FIG. 9A, inulin) and encapsulated (FIG. 9B, encapsulated) carbohydrate. For example, an aqueous solution of 10% (w/w) inulin and 20% (w/w) glucose is prepared, followed by the addition of 3% (w/w) agarose powder. The mixture is heated to 75° C. to dissolve the agarose, which, upon cooling, forms a solid. Unlike the loose powder depicted in FIG. 9A, the formulations generated using the methods of manufacture outlined are solid, cohesive particle preparation(s) (11B), indicating successful encapsulation.

In one non-limiting example, encapsulated carbohydrate satiety modulating composition(s) demonstrate controlled release. For example, FIG. 9C illustrates a carbohydrate release from a formulation containing encapsulated inulin. In this example, inulin releases from the exemplary satiety modulating composition(s) (5% (w/w) agarose, 0.2% (w/w) locust bean gum, 5% (w/w) calcium caseinate, and 1% (w/w) inulin), reaching 100% release by 24 hours in phosphate buffered saline, pH 7.4.

In certain embodiments, one or more satiety modulator(s) characterized as a ketone is or are encapsulated in one or more core-shell preparations. In certain embodiments, the encapsulation of one or more ketone(s) in one or more core-shell preparation(s) is further characterized by the release of encapsulated ketone(s) in one or more release environment(s). In some instances, the release profile(s), as provided herein, of ketone(s) encapsulated in one or more core-shell preparation(s) illustrate controlled release of ketone(s). In some embodiments, 3-hydroxybutyrate, acetoacetic acid, and/or 3-hydroxybutyl-3-hydroxybutyrate may be encapsulated and exhibit controlled release within and from provided satiety modulating composition(s), respectively.

### I. Example 9: Cross-Sectional Morphology of One or More Satiety Modulating Composition(s)

In certain embodiments of the present disclosure, one or more satiety modulating composition(s) is further characterized as core-shell preparation(s). In some non-limiting embodiments, microscopy is useful to illustrate the micro-scale morphology of one or more core-shell preparation(s). For example, in some instances, microscopy is or may be useful to quantify and/or qualify gross characteristics, surface characteristics, and/or cross-sectional characteristics of one or more satiety modulating composition(s).

In some non-limiting embodiments, quantification and/or qualification of gross characteristics is or may refer to shape, morphology, and/or diameter of one or more particle preparation(s). In some non-limiting embodiments, quantification and/or qualification of surface characteristics is or may refer to color, texture, and/or extent of surface coating of one or more particle preparation(s). In some non-limiting embodiments, quantification and/or qualification of cross-sectional characteristics is or may refer to coating thickness, spatial arrangement, homogeneity, and/or porosity of one or more particle preparation(s).

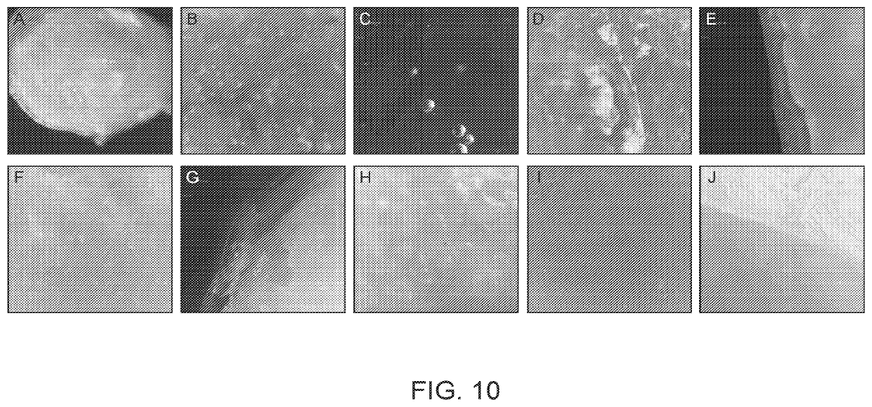

In some non-limiting embodiments, depicted in FIG. 10, cross-sectional micrographs of one or more satiety modulating composition(s) enables quantification and/or qualification of gross, surface, and/or cross-sectional characteristics. For example, the spatial arrangement, coating thickness, and extent of surface coating of a core-shell preparation comprising sucrose, amylose, and Zein are clearly illustrated in the micrograph of FIG. 10A. For example, the porosity of a core component comprising inulin and alginate is clearly illustrated in the micrograph of FIG. 10B. For example, the color, homogeneity, and lack of porosity of a core component comprising inulin and agarose are clearly illustrated in the micrograph of FIG. 10C. For example, the spatial arrangement, porosity, and color of a core-shell preparation comprising a core component further characterized as a matrix of glucose, agarose, and inulin, coated with a shell component of cellulose acetate phthalate are clearly illustrated in the micrograph of FIG. 10D. For example, the spatial arrangement, color, extent of coating, and coating thickness of a core-shell preparation comprising a core component further characterized as a matrix of whey protein isolate and fully hydrogenated soy oil, coated with a shell component of cellulose acetate phthalate are clearly illustrated in the micrograph of FIG. 10E. For example, the color, homogeneity, and lack of porosity of a matrix formulation comprising whey protein isolate and beeswax are clearly illustrated in the micrograph of FIG. 10F. For example, the spatial arrangement, extent of coating, and coating thickness of a core-shell preparation comprising a core component further characterized as a matrix of whey protein isolate and agarose, coated with a shell component of cellulose acetate phthalate are clearly illustrated in the micrograph of FIG. 10G. For example, the color and homogeneity of a core component comprising oleic acid and ethyl cellulose are clearly illustrated in the micrograph of FIG. 10H. For example, the color and homogeneity of a core component comprising oleic acid and carnauba wax are clearly illustrated in the micrograph of FIG. 10I. For example, the color, homogeneity, lack of porosity, spatial arrangement, extent of coating, and coating thickness of a core-shell preparation comprising a core component further characterized as a core component comprising whey protein isolate, oleic acid, ethyl cellulose, and carnauba wax encapsulated in a shell component of cellulose acetate phthalate, are clearly illustrated in the micrograph of FIG. 10J.

### J. Example 10: Exemplary Coating Formulation Protocols

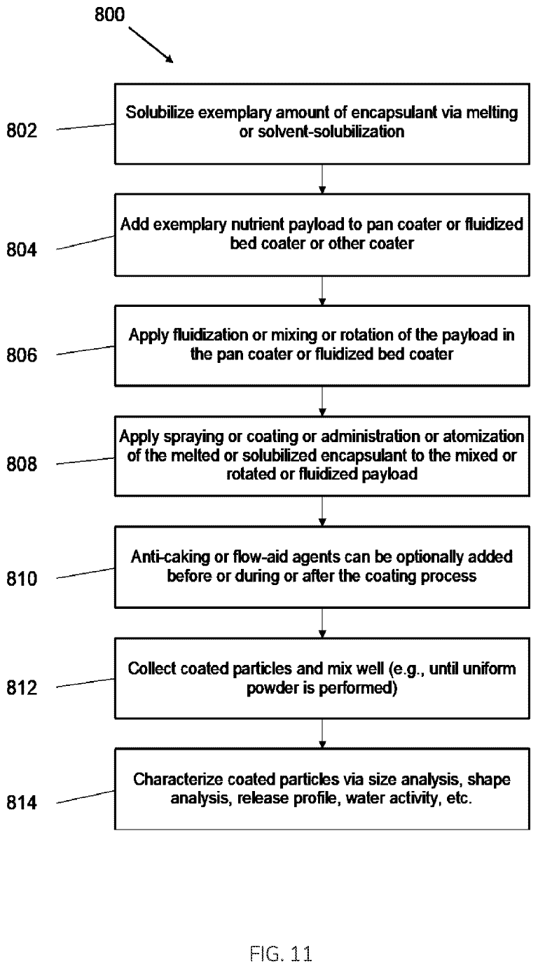

This example describes two non-limiting processes of arranging one or more satiety modulator(s) as a shell component to one or more satiety modulator(s) characterized as a core component via spray pan coating and/or fluidized bed spray coating. A schematic of an exemplary coating procedure, method, or protocol 800 is presented in FIG. 11. At step 802, the method 800 may include solubilizing an exemplary amount of encapsulant via melting or solvent-solubilization. At step 804, the method 800 may include adding an exemplary nutrient payload to pan coater or fluidized bed coater or other coater. At step 806, the method 800 may include applying fluidization or mixing or rotation of the payload in the pan coater or fluidized bed coater. At step 808, the method 800 may include applying spraying or coating or administration or atomization of the melted or solubilized encapsulant to the mixed, rotated and/or fluidized payload. At step 810, the method 800 may include adding anti-caking or flow-aid agents before, during and/or after the coating process. At step 812, the method 800 may include collecting the coated particles and thoroughly mixing (e.g., until uniform powder is achieved). At step 814, the method 800 may include characterizing the coated particles via size analysis, shape analysis, release profile, water activity, etc.

In one non-limiting example, satiety modulating composition(s) are prepared in core-shell preparations using the procedure described below. Particle preparations comprising sucrose encapsulated in amylose (10 g) are coated using a spray pan coater with an inlet air temperature of 80° C., pan temperature of 70° C., rotation speed of 2 Hz, and spray rate of 0.5 mL/s. A 10% (w/v) ethanolic (90% ethanol) solution of Zein with 1% (v/v) Propylene Glycol and 0.5% (w/v) Talc powder is applied as a thin film over 5 minutes to the encapsulated sucrose. The volume-normalized weight gain due to coating is 120%. The concentration of formulated satiety modulator in this embodiment, on a dry weight basis, is 100% (w/w).

Whey protein isolate powder (100 g) is coated using a fluidized bed spray coater (Glatt) with a 67° C. inlet temperature, 45° C. outlet temperature, a spray rate of 3 g/min, and a flow rate of 25 mL/min. A 20% (w/v) aqueous suspension of ethyl cellulose is applied as a thin film over 5 min to the fluidized whey protein powder. The volume-normalized weight gain due to coating is 110%. The concentration of formulated satiety modulator in this embodiment is 100% (w/w).

### K. Example 11: Core-Shell Preparation(s) as a Means of Controlling the Release of One or More Satiety Modulator(s)

In certain embodiments, the spatial arrangement of one or more satiety modulator(s) establishes a means of controlling the release of one or more satiety modulator(s). In certain embodiments, the spatial arrangement of one or more satiety modulator(s) comprising one or more satiety modulating composition(s) is further characterized as a core component and/or a core-shell preparation. In some non-limiting embodiments, the spatial arrangement of one or more satiety modulating composition(s) further characterized as a core-shell preparation establishes a means of controlling the release of one or more satiety modulator(s) from one or more satiety modulating composition(s).

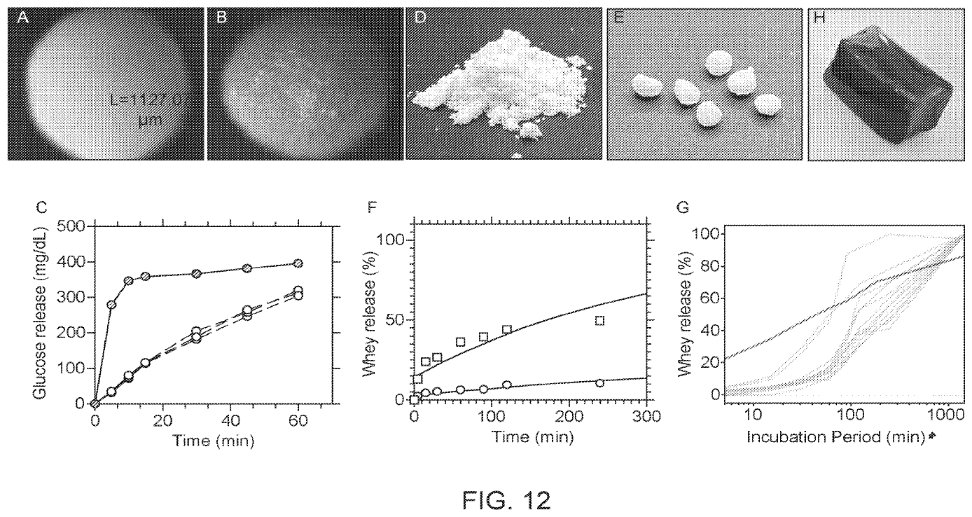

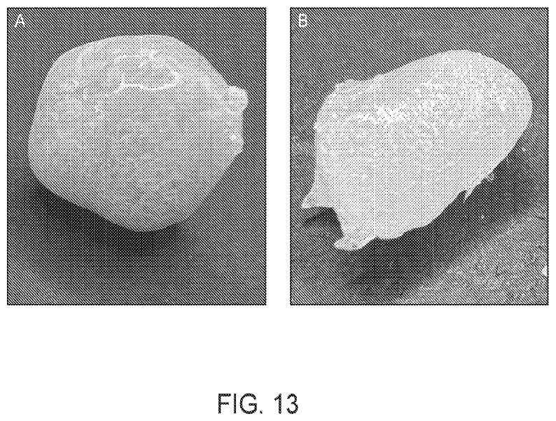

For example, in one non-limiting embodiment, a satiety modulating composition comprised of a monosaccharide (e.g., sucrose) is embedded within a polysaccharide (e.g., amylose), further characterized as a core component. This core component, depicted in FIG. 12A, is subsequently coated with a solution of 10% (w/v) Zein, 1.5% (w/v) propylene glycol, and 2% (w/v) glycerol monostearate in a 90% (v/v) ethanolic solution using the methodology outlined in Example 9. Without wishing to be bound by any particular theory, it is contemplated that the applied shell component(s) comprising Zein, depicted in FIG. 12B, confer water-resistance towards one or more satiety modulating composition(s) and reduce release. As shown in FIG. 12C, Zein-coated sucrose particle preparation(s) (denoted by empty circles) exhibit slower release than uncoated sucrose particle preparation(s) (denoted by filled circles) over a 60 minute period in 10 mM phosphate buffered saline pH 7.4.

For example, in one non-limiting embodiment, a satiety modulating composition comprised of a protein (e.g., whey protein isolate, FIG. 12D) is encapsulated within a polysaccharide (e.g., chitosan polyphosphate), further characterized as a core-shell preparation (FIG. 12E). In this non-limiting example, a 1.25% (w/v) chitosan solution is prepared in 1% acetic acid in distilled water, followed by the addition of 10% (w/v) whey protein isolate. The resulting viscous solution is added dropwise to a 30% (w/v) solution of sodium hexametaphosphate at room temperature; formed particles are allowed to cross-link for 10 minutes and separated from the aqueous medium by filtration. The gelatinous chitosan polyphosphate particles comprising whey protein isolate are subsequently dried at 50° C. for 4 hours to yield hard, spherical particles (FIG. 12E). As shown in FIG. 12F, a shell component comprising chitosan polyphosphate exhibits slower release of encapsulated whey protein isolate in simulated gastric fluid (filled circles) vs simulated intestinal fluid (filled squares).

For example, in one non-limiting embodiment, satiety modulating composition(s) comprised of a protein (e.g., whey protein isolate, FIG. 12D) is encapsulated within a polysaccharide (e.g., agarose), further characterized as a core component. In this example, to a 20% (w/v) aqueous solution of the exemplary protein at 50° C. is added an equal portion of 6% (w/v) agarose solution at 75° C. The mixture is briefly homogenized at 50° C. before pouring into a mold. Subsequently, the gelatinous formulation is coated using an ethanolic solution of Zein with additives (e.g., chitosan, poly(vinyl acetate), citric acid esters of diglycerides) as provided in Example 9, thus demonstrating encapsulation of a protein within a protein, a carbohydrate, and/or a polymer. As shown in FIG. 12G, the result core-shell preparation(s) (light grey) delay the release of whey protein relative to uncoated whey/agarose core component(s) (dark grey).

For example, in one non-limiting embodiment, satiety modulating composition(s) comprised of a protein (e.g., whey protein isolate, FIG. 12D) is encapsulated within a polysaccharide (e.g., agarose), further characterized as a core component. Subsequently, the gelatinous formulation is coated using an ethanolic solution of Zein with additives (e.g., linoleic acid, cyanidin chloride) as provided in Example 9, thus demonstrating encapsulation of a protein within a protein, a lipid, and/or a flavonoid.

### L. Example 12: Exemplary Core-Shell Preparations

In certain embodiments, one or more satiety modulating composition(s) are characterized as one or more of a core-shell preparation and/or particle preparation.

For example, in one non-limiting embodiment, a satiety modulating composition is comprised of a core component, coated with one or more shell components. In one non-limiting embodiment, one or more core component(s) is comprised of, on a dry weight basis, at least 90% of one or more satiety modulator(s).

In one non-limiting embodiment, one or more core component(s) comprising oleic acid, ethyl cellulose, carnauba wax, and whey protein isolate (FIG. 13A) is coated 4 times with a 15% (w/v) solution of cellulose acetate phthalate in acetone. The resulting exemplary core-shell preparation exhibits a smooth, reflective, and non-tacky surface indicative of complete and successful coating.

In one non-limiting embodiment, one or more core component(s) comprising glucose and pectin (FIG. 13B) is coated 4 times with a 15% (w/v) solution of cellulose acetate phthalate in acetone. The resulting exemplary core-shell preparation exhibits a smooth, reflective, and non-tacky surface indicative of complete and successful coating.

### M. Example 13: Exemplary Dissolution Protocol

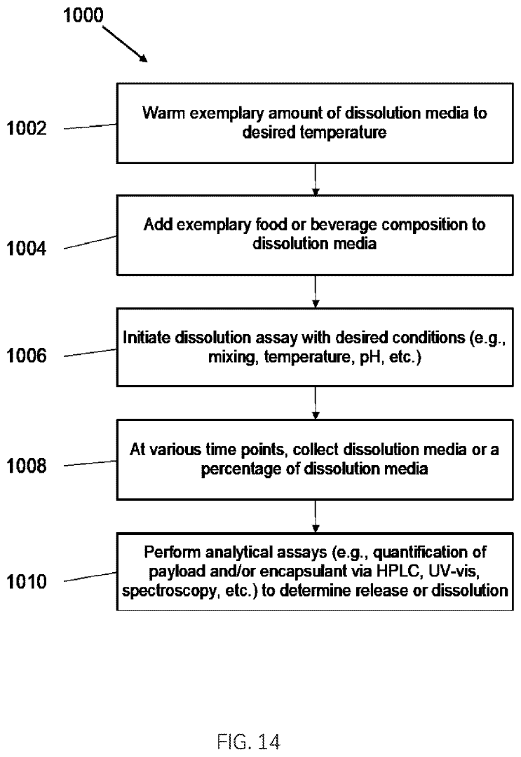

This example describes one non-limiting process of assessing the release (e.g., controlled release) of one or more satiety modulator(s) from one or more dissolution solvent(s). A schematic of an exemplary dissolution procedure, method, or protocol 1000 is presented in FIG. 14. At step 1002, the method 1000 may include warming an exemplary amount of dissolution media to a desired temperature. At step 1004, the method 1000 may include adding an exemplary satiety modulating composition to the dissolution media. At step 1006, the method 1000 may include initiating dissolution assay with one or more desired conditions (e.g., via mixing, temperature, pH, etc.). At step 1008, the method 1000 may include collecting dissolution media and/or a percentage of dissolution media at various time points. At step 1010, the method 1000 may include performing analytical assays (e.g., quantification of payload and/or encapsulant via HPLC, UV-vis, spectroscopy, etc.) to determine release or dissolution characteristics.

In one non-limiting example, a satiety modulating composition characterized as a core-shell preparation (e.g., Zein-coated amylose encapsulating sucrose) is assessed for controlled release in an aqueous dissolution solvent (e.g., 10 mM phosphate buffered saline, pH 7.4). 12 mL of 10 mM phosphate buffered saline are added to a polypropylene 15 mL centrifuge tube and allowed to equilibrate for 30 min while rotating at 10 rpm on a laboratory rotator. Exemplary core-shell preparations comprising Zein, sucrose, and amylose (500 mg) are added to the rotating tube. 100 μL aliquots are sampled at time points of 1 minute, 2 minutes, 5 minutes, 10 minutes, 15 minutes, 20 minutes, 30 minutes, 40 minutes, 50 minutes, and 60 minutes following addition of core-shell preparations and stored in 1.5 mL centrifuge tubes. The concentration of sucrose in collected aliquots is assayed using an enzymatic electrochemical method. Sucrose concentration, mass, and percent release is plotted with respect to incubation period to construct a release profile.

### N. Example 14: Release of Satiety Modulator(s) in One or More Release Environment(s)

In certain embodiments, one or more satiety modulator(s) are released from one or more satiety modulating composition(s) in one or more release environment(s). In some instances, one or more release environment(s) provides similar pH, ionic strength, solubilizers, and/or chemical components as in one or more biological compartment(s). In some instances, the release of one or more satiety modulator(s) from one or more satiety modulating composition(s) in one or more release environment(s) predicts the release of one or more satiety modulator(s) from one or more satiety modulating composition(s) in one or more biological compartment(s).

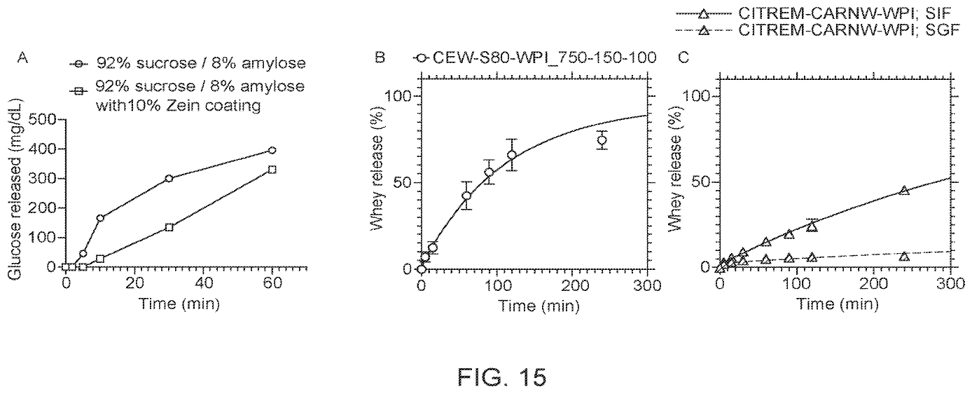

For example, in one non-limiting embodiment, the release of glucose from one or more satiety modulating composition(s) is measured in 10 mM phosphate buffered saline solution at pH 7.4 with 1% (w/v) hydroxypropyl methylcellulose over a 60-minute period (FIG. 15A). For example, in one non-limiting embodiment, the release of whey protein isolate from one or more satiety modulating composition(s) is measured in 10 mM phosphate buffered saline at pH 7.4 over a 240-minute period (FIG. 15B). For example, in one non-limiting embodiment, the release of whey protein isolate from one or more satiety modulating composition(s) is measured in simulated intestinal fluid (i.e., SIF) and simulated gastric fluid (i.e., SGF) over a 240-minute period (FIG. 15C).

### O. Example 15: Exemplary Control of Release of One or More Satiety Modulator(s) by One or More pH-Responsive Satiety Modulator(s)

As provided herein, the spatial arrangement of one or more satiety modulator(s) in one or more satiety modulating composition(s) establishes a means of controlling the release of one or more satiety modulator(s). In some embodiments, the spatial arrangement of one or more satiety modulating composition(s) is further characterized as a core-shell preparation. In some embodiments, the shell component(s) of one or more core-shell preparation(s) establishes a means of controlling the release of one or more satiety modulator(s). In some instances, the shell component(s) of one or more core-shell preparation(s) are further comprised of pH-responsive satiety modulator(s).

As provided herein, one or more pH-responsive satiety modulator(s) comprising the shell component(s) of one or more non-limiting core-shell preparation(s) provide for increased and/or decreased release of one or more satiety modulator(s) in response to the pH of one or more release environment(s). For example, in some instances, one or more pH-responsive satiety modulator(s) may increase the release of one or more satiety modulator(s) in simulated intestinal fluid and/or simulated gastric fluid. For example, in some instances, one or more pH-responsive satiety modulator(s) may decrease the release of one or more satiety modulator(s) in simulated intestinal fluid and/or simulated gastric fluid.

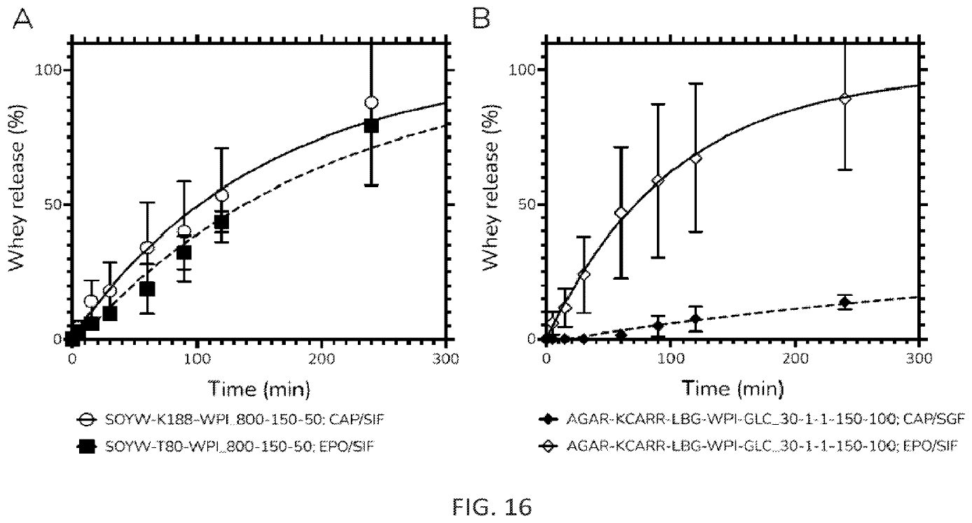

In one non-limiting example, one or more satiety modulating composition(s) further characterized as a core-shell preparation wherein the shell component(s) comprise cellulose acetate phthalate exhibit responsiveness, and concomitant whey protein isolate release, to the pH of simulated intestinal fluid (FIG. 16A). Coating of the same satiety modulating composition(s) with Eudragit E PO exhibit resistance, and concomitant reduction of whey protein isolate release relative to that of cellulose acetate phthalate-coated satiety modulating composition(s), to the pH of simulated intestinal fluid (FIG. 16A).

In one non-limiting example, one or more satiety modulating composition(s) further characterized as a core-shell preparation wherein the shell component(s) comprise cellulose acetate phthalate exhibit resistance, and concomitant reduction of whey protein isolate release, to the pH of simulated gastric fluid (FIG. 16B).

### P. Example 16: Exemplary Theoretical Release Profiles of Satiety Modulator(s)

As provided herein, one or more satiety modulating and/or beverage composition(s) is characterized by controlled release of one or more satiety modulator(s). Selection of release profile, as described herein, is intended to confer a benefit (as described herein) one or more animal(s). The following example depicts anticipated (e.g., theoretical) non-limiting release profiles exhibited by one or more satiety modulating composition(s).

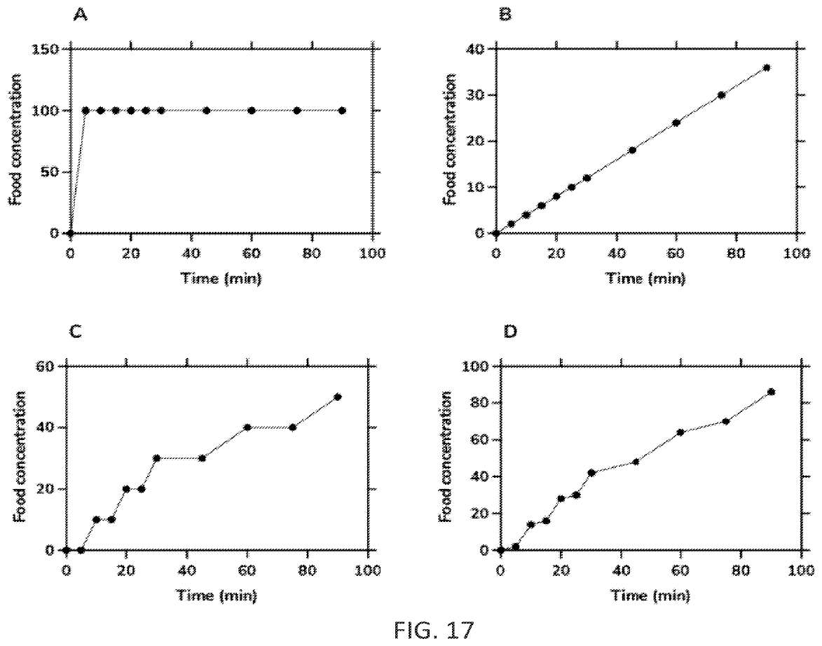

In one non-limiting example, the release of one or more satiety modulator(s) is characterized as a single bolus release, as measured by concentration present in one or more dissolution solvent(s) over time, as shown in FIG. 17A. In one non-limiting example, the release of one or more satiety modulator(s) is characterized as release with constant rate, as measured by concentration present in one or more dissolution solvent(s) over time, as shown in FIG. 17B. In one non-limiting example, the release of one or more satiety modulator(s) is characterized as multiple bolus dose (e.g., pulsatile) release, as measured by concentration present in one or more dissolution solvent(s) over time, as shown in FIG. 17C. In one non-limiting example, the release of one or more satiety modulator(s) is characterized as a combination of multiple bolus dose and constant release rate, as measured by concentration present in one or more dissolution solvent(s) over time, as shown in FIG. 17D.

### Q. Example 17: One or More Satiety Modulator(s) Establishes a Means of Controlling the Release of One or More Satiety Modulator(s)

Among other things, the present disclosure provides one or more means of controlling the release of one or more satiety modulator(s) from one or more satiety modulating composition(s). In some instances, the spatial arrangement of one or more satiety modulator(s) provides one or more means of controlling the release of one or more satiety modulator(s) from one or more satiety modulating composition(s). In some instances, the selection of one or more satiety modulator(s) release of one or more satiety modulator(s) from one or more satiety modulating composition(s).

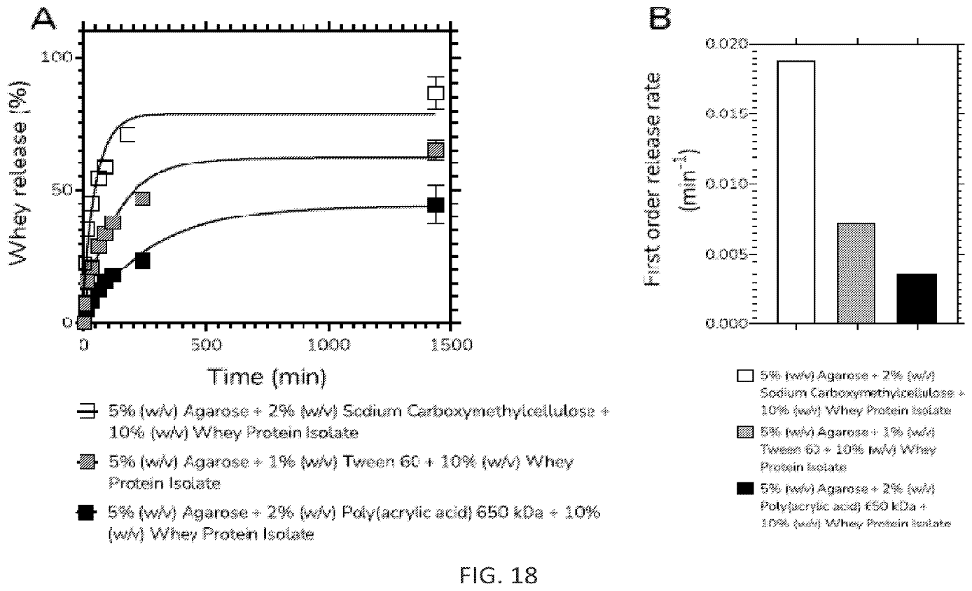

As illustrated in one non-limiting example, one or more satiety modulating composition(s) further characterized as core components comprising 5% (w/v) agarose and 10% (w/v) whey protein isolate exhibit substantial differences in protein release depending on included satiety modulator(s) (FIG. 18A). For example, these exemplary uncoated core components further comprising 2% (w/v) sodium carboxymethylcellulose (white squares) exhibit nearly 75% release of loaded whey protein isolate over 24 hours, while preparations comprising 1% (w/v) Tween 60 (grey squares) or 2% (w/v) poly(acrylic acid) (black squares) exhibit only 60% and 45% release at 24 hours, respectively. This example demonstrates the importance of satiety modulator selection in tuning the controlled release of one or more satiety modulator(s) from one or more satiety modulating composition(s).

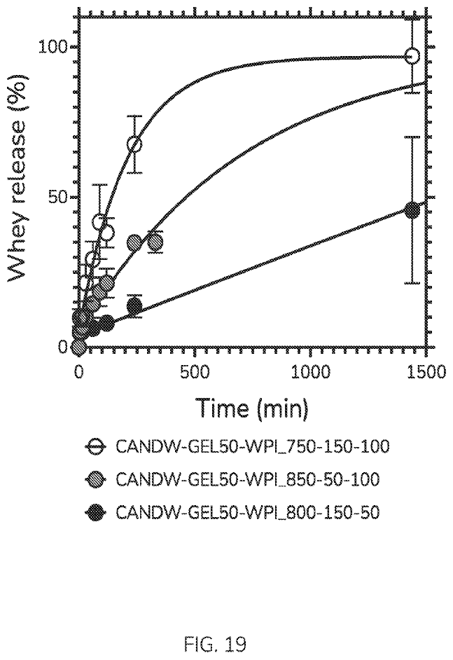

As illustrated in one non-limiting example, one or more satiety modulating composition(s) further characterized as core components comprising candelilla wax, gelucire 50/13 and whey protein isolate exhibit substantial differences in protein release depending on the selected concentration(s) of satiety modulator(s) (FIG. 19). For example, these exemplary uncoated core components further comprising 75% (w/v) candelilla wax (white circles) exhibit nearly 100% release of loaded whey protein isolate over 24 hours, while preparations comprising 85% (w/v) (grey circles) or 80% (w/v) candelilla wax (black circles) exhibit only 80% and 45% release at 24 hours, respectively. This example demonstrates the importance of satiety modulator(s) concentration in tuning the controlled release of one or more satiety modulator(s) from one or more satiety modulating composition(s).

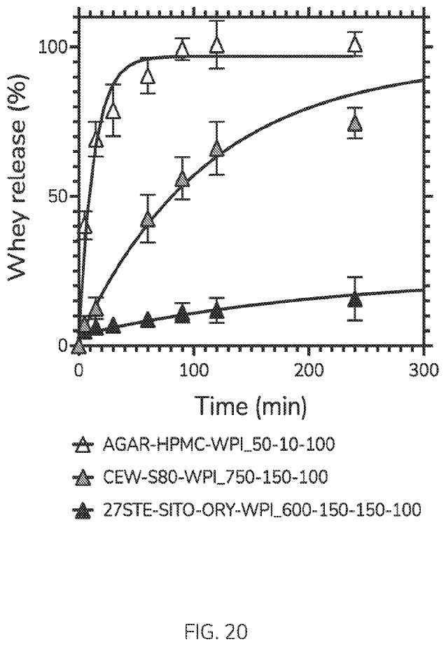

As illustrated in one non-limiting example, one or more satiety modulating composition(s) further characterized as core components exhibit substantial differences in protein release depending on the selected core component(s) (FIG. 20). For example, these exemplary core components further comprising an agarose-based matrix (white triangles) exhibit nearly 100% release of loaded whey protein isolate over 240 minutes, while core components comprising lipid and surfactant (grey triangles) or oleogels (black triangles) exhibit only 80% and 45% release at 24 hours, respectively. This example demonstrates the importance of selecting satiety modulator(s) as core component(s) in tuning the controlled release of one or more satiety modulator(s) from one or more satiety modulating composition(s) further characterized as a core-shell preparation.

### R. Example 18: Satiety Modulating Compositions Exhibiting Low Water Activity and Moisture Content

The presence of water and/or water activity is a common factor underlying instability in one or more satiety modulating composition(s). The following example illustrates the ability of one or more satiety modulators in the provided satiety modulating compositions to retain integrity in high-moisture conditions, resist water uptake, and thereby mitigate instability of the satiety modulator(s) included therein.

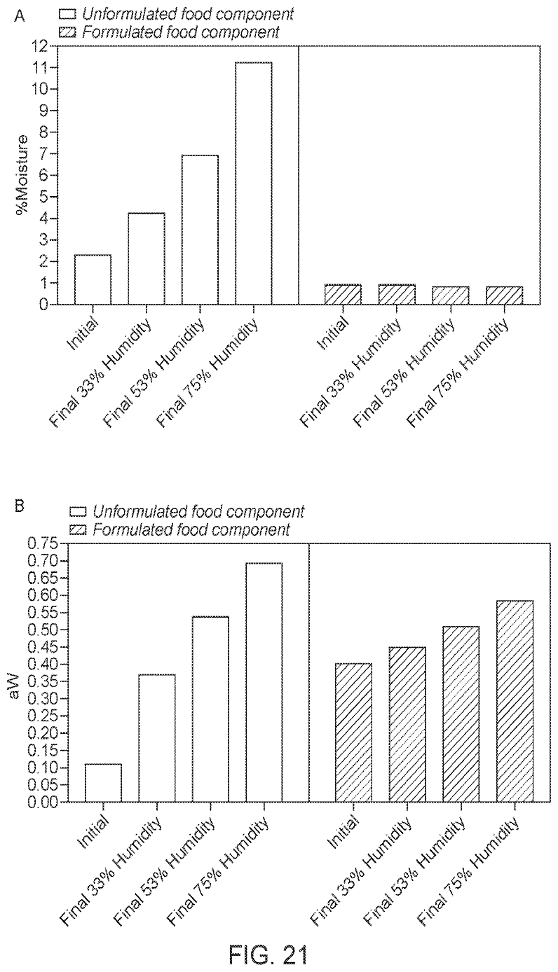

For example, FIG. 21A demonstrates that satiety modulating compositions herein provided do not gain moisture content, even when exposed to controlled relative humidity of 33%, 53%, or 75% for 4 days. Unformulated satiety modulating (e.g., dehydrated milk powder), on the other hand, demonstrates a 2-5 fold increase in moisture content. FIG. 21B reveals that formulated satiety modulating compositions exhibit a smaller increase in water activity as compared to un-encapsulated satiety modulating. For example, even when the initial level of water activity is higher, as shown in FIG. 21B, the encapsulated satiety modulating compositions demonstrate a lower level of water activity increase when exposed to increasing amounts of humidity. As such, even when exposed to 75% relative humidity, the water activity of the exemplary satiety modulating compositions demonstrate lower water activity levels than un-encapsulated satiety modulating,

### S. Example 19: Incorporation of Satiety Modulating Composition(s) into Food and/or Beverage Products

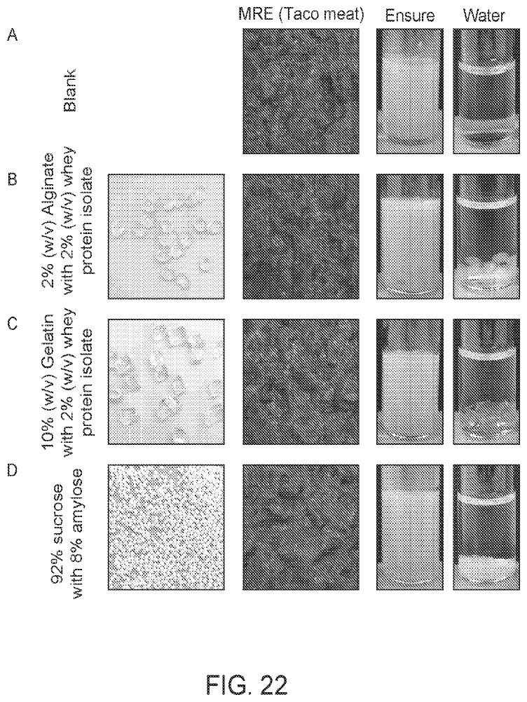

This example illustrates homogeneous mixtures of disclosed satiety modulating composition(s) within food and/or beverage products (e.g., MRE, nutritional beverage, water) as demonstrated in FIGS. 22A-D. It is contemplated that non-limiting exemplary embodiments of satiety modulating compositions can be homogeneously mixed with other satiety modulators such as freeze dried powder, protein powder, solid bars, domestic pet satiety modulating (pellets), liquid shakes, pudding, etc. Homogenization can be achieved without additional processing aid or improved through addition of processing aid/excipients, through the use of mixing apparatuses such as a homogenizer, stand mixer, paddle blender, stir bar, spatula, etc. Without wishing to be bound by any particular theory, the present disclosure proposes that size characteristics and/or compositions of certain provided satiety modulating composition(s) may surprisingly contribute desirable and/or useful attribute(s) to such particles, specifically including, for example, amenability to homogenous combination with other component(s). As shown in FIGS. 22B-D, incorporation of alginate beads, gelatin beads, each encapsulating whey protein isolate, and/or sucrose-encapsulating beads into MRE and Ensure is homogeneous and associated with minimal change in visual appearance. In certain embodiments, incorporation of satiety modulating composition(s) within one or more food and/or beverage products is associated with structural changes. In one non-limiting example (FIGS. 22A-D), satiety modulating composition(s) are shown to change morphology over a 1-hour incubation period, with gelatin and alginate beads exhibiting expansion and sucrose-encapsulating beads exhibiting dissolution.

### T. Example 20: Exemplary Protocol for Core Component(s) Comprising One or More Fat(s), Protein(s) and Acaloric Phytonutrient(s)

The following non-limiting example describes the preparation of provided satiety modulating composition(s) further characterized as core component(s) comprising one or more fat(s), one or more protein(s), and/or one or more acaloric phytonutrient(s).

100 g of oleic acid was first added to a 500 mL beaker and heated to 130° C., or until all solids were completely melted. 15 mL of molten oleic acid was transferred to a 150 mL beaker held at 120° C., stirring at 300 RPM. 3 mL of 10% (w/w) chrysin solution was transferred to the beaker and the solution was stirred for 5 minutes at 120° C. to ensure thorough mixing. Homogenization was visually confirmed via color change from clear to yellow of the solution. 2 g of whey protein isolate was added and the solution was stirred for 15 seconds at 120° C. and 300 rpm to fully disperse the added powder. The stirring suspension was poured into an aluminum pan and allowed to set at 20° C. for 1 hour. Solid formulations were segmented into sections of approximately 500 mg for future dissolution assays.

### U. Example 21: Exemplary Protocol for Quantification of Release of Protein(s) from One or More Satiety Modulating Composition(s)

Tubes filled with 12 mL of 10 mM phosphate buffered saline (PBS), pH were added to a rotating incubator set at 37° C. An initial 100 μL sample was collected from each tube and transferred into a 96 well polypropylene plate. Then, each 500 mg satiety modulating composition(s) was placed into its corresponding tube and the timer was promptly started. 100 μL samples were collected from the warmed, rotating tubes at subsequent timepoints of 5, 15, 30, 60, 90, 120, and 240 minutes. A BCA reagent mixture was prepared with reagent A (23228, Thermo Scientific Pierce, Waltham, MA) and reagent B (23224, Thermo Scientific Pierce, Waltham, MA) in a 50:1 ratio. 25 μL of each sample or standard was pipetted into a 96 well polystyrene plate followed by addition of 200 μL of BCA reagent mixture. The plate was then incubated for 25 minutes at 37° C. followed by measurement of absorbance at 567 nm.

### V. Example 22: Exemplary Protocol for Quantification of Release of Protein(s) from One or More Satiety Modulating Composition(s)

Tubes filled with 12 mL of 10 mM phosphate buffered saline (PBS), pH were added to a rotating incubator set at 37° C. An initial 100 μL sample was collected from each tube and transferred into a 96 well polypropylene plate. Then, each 500 mg satiety modulating composition(s) was placed into its corresponding tube and the timer was promptly started. 100 μL samples were collected from the warmed, rotating tubes at subsequent timepoints of 5, 15, 30, 60, 90, 120, and 240 minutes. A BCA reagent mixture was prepared with reagent A (23228, Thermo Scientific Pierce, Waltham, MA) and reagent B (23224, Thermo Scientific Pierce, Waltham, MA) in a 50:1 ratio. 25 μL of each sample or standard was pipetted into a 96 well polystyrene plate followed by addition of 200 μL of BCA reagent mixture. The plate was then incubated for 25 minutes at 37° C. followed by measurement of absorbance at 567 nm.

### W. Example 23: Exemplary Protocol for Quantification of Release of Carbohydrate(s) from One or More Satiety Modulating Composition(s)

Tubes filled with 12 mL of 10 mM phosphate buffered saline (PBS), pH were added to a rotating incubator set at 37° C. An initial 100 μL sample was collected from each tube and transferred into a 96 well polypropylene plate. Then, each 500 mg satiety modulating composition(s) was placed into its corresponding tube and the timer was promptly started. 100 μL samples were collected from the warmed, rotating tubes at subsequent timepoints of 5, 15, 30, 60, 90, 120, and 240 minutes. An Amplex Red reagent mixture was prepared by mixing 4.75 mL of 50 mM phosphate buffer pH 7.4, 100 μL of 10 U/mL horseradish peroxidase, 100 μL of 100 U/mL glucose oxidase, and 50 μL of 2.5 mg/mL Amplex Red in DMSO. 50 μL of each sample or standard was pipetted into a 96 well polystyrene plate followed by addition of 50 μL of Amplex Red reagent mixture. The plate was then incubated for 25 minutes at 37° C. followed by measurement of fluorescence with excitation of 565 nm and emission of 590 nm.

### X. Example 24: Exemplary Formulations

Non-limiting exemplary embodiments in accordance with the present disclosure (e.g., exemplary formulations, e.g., exemplary compositions) are presented in Appendix A, the entire contents of which are hereby incorporated by reference.

### Y. Example 25: Exemplary Multiple-Layer Core-Shell Preparation Establishing a Means of Controlling Satiety Modulator Release

The following non-limiting example demonstrates one or more multiple-layer core-shell preparations comprising one or more protein(s) and one or more polysaccharide(s). In an unexpected result, the spatial orientation of one or more layer(s) comprising one or more core-shell preparation(s) was found to be a means of controlling the release of one or more protein(s) from one or more core-shell preparation(s). Moreover, in an unexpected result, an inner shell comprising an exemplary polysaccharide Hypromellose, with a viscosity of 100 cP, and outer shell comprising a different exemplary polysaccharide, Ethyl cellulose, was found to enable faster release than an inner shell comprising an exemplary polysaccharide, Ethyl cellulose, and an outer shell comprising Hypromellose.

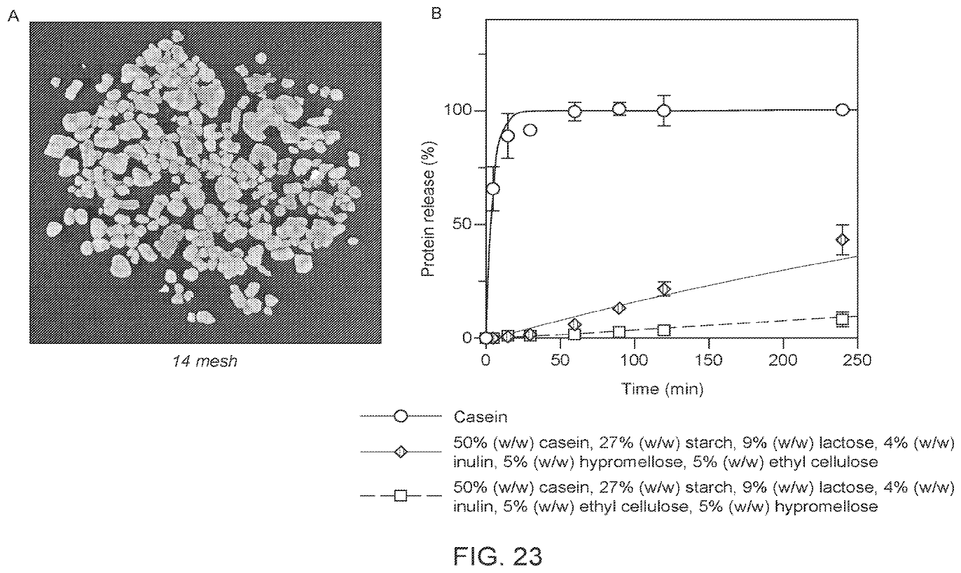

In this non-limiting example, a powdered mixture of 55% micellar casein, 30% directly compressible starch, 10% lactose, and 5% inulin was granulated using a Caleva Multi-Lab, followed by extrusion and spheronization via 1 mm×1 mm dies with constant addition of Dry-Flo starch. The resulting spherical particles were dried in an oven at 45° C. for 16 hours and passed through a 14-mesh sieve. Individual free particles were weighed and loaded into a VFC-LAB Micro FLO-COATER (Freund Vector) and sprayed with either a 10% (w/v) Ethyl cellulose in ethanol solution followed by a 2% (w/v) Hypromellose solution in water (Coating A) or a 2% (w/v) Hypromellose solution in water followed by a 10% (w/v) Ethyl cellulose in ethanol solution (Coating B). In each case, a 5% coating weight gain was targeted for each layer, resulting in a total 10% weight gain coating. The final concentration of all constituents in the particle preparation was 50% (w/w) casein, 27% (w/w) starch, 9% (w/w) lactose, 4% (w/w) inulin, 5% (w/w) Hypromellose, and 5% (w/w) ethyl cellulose. Nine (9) 15 mL centrifuge tubes were filled with PBS at 37° C. To 3 tubes each, was added either 55 mg of micellar casein, 100 mg of casein particles with Coating A, or 100 mg of casein particles with Coating B. As shown in FIG. 23A, formulated core-shell particles comprising protein exhibit a spherical morphology with a 14-mesh size. As shown in FIG. 23B, the release of unformulated micellar casein is rapid in PBS, with nearly complete release after 5 minutes. In contrast, protein particles with either Coating A or Coating B substantially delay release with less than 50% release even after 4 hours of incubation with PBS. Importantly, Coating B was found to delay release relative to Coating A, despite being composed of the same materials, indicating that spatial orientation of shell(s) is a means of controlling payload release.

### Z. Example 26: Exemplary Core Component(s) Exhibiting pH-Controlled Release of Protein(s)

The following non-limiting example demonstrates one or more satiety modulating composition(s) comprising one or more core component(s) further comprising one or more protein(s) and one or more polysaccharide(s) characterized as pH-responsive. As provided herein, one or more pH-responsive polysaccharide(s) exhibits one or more change(s) upon exposure to varying pH. In the following non-limiting example, sodium alginate is one or more pH-responsive polysaccharides.

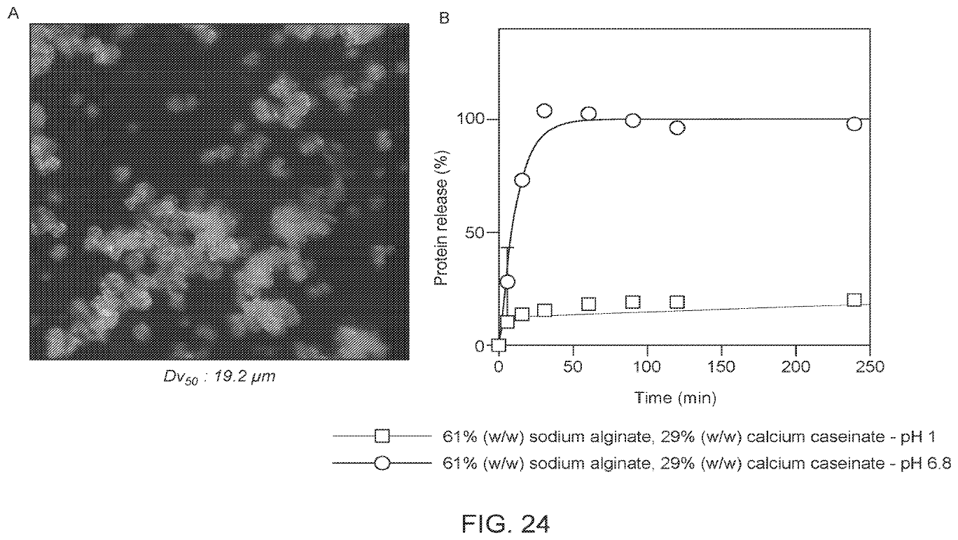

In this non-limiting example, a suspension comprising 2% (w/v) sodium alginate, 4% (w/v) calcium caseinate, 0.15% (w/v) calcium hydrogen phosphate, and 1% (w/v) succinic acid in distilled water was prepared and the pH was adjusted to 8 using ammonium hydroxide. In an unexpected result, passing said suspension through a Buchi B-290 Spray Dryer equipped with an ultrasonic nozzle with an inlet temperature of 90° C., nozzle temperature of 50° C., and outlet temperature of 40° C. yielded solid spherical particles (FIG. 24A) comprising sodium alginate and calcium caseinate. Particle size analysis of the spherical particles indicated an average particle diameter, Dv₅₀, of 19.2 μm. Three (3) 15 mL centrifuge tubes were filled with simulated gastric fluid (SGF) at 37° C., with a pH of 1. Three (3) 15 mL centrifuge tubes were filled with simulated intestinal fluid (SIF) at 37° C., with a pH of 6.8. 60 mg of spray-dried casein-containing particles was added to each tube. As shown in FIG. 24B, the release of casein from these particles was rapid in SIF, with complete release after only 20 minutes; in contrast, less than 25% of encapsulated casein was released from these particles even after more than 4 hours of incubation in SGF.

### AA. Example 27: Exemplary Core Component(s) Exhibiting Sustained Release of Fatty Acid in One or More Release Environment(s)

The following non-limiting example demonstrates one or more core component(s) comprising one or more fatty acid(s) and one or more lipid(s). As provided herein, one or more release environment(s) is or may be comprised of one or more component(s) simulating digestive condition(s) of the gastrointestinal tract of one or more mammal(s). Without wishing to be bound by any particular theory, it is contemplated that the release and/or absorption of one or more payload(s) further characterized as one or more fatty acid(s) is mediated by bile salt(s), for example, sodium taurocholate.

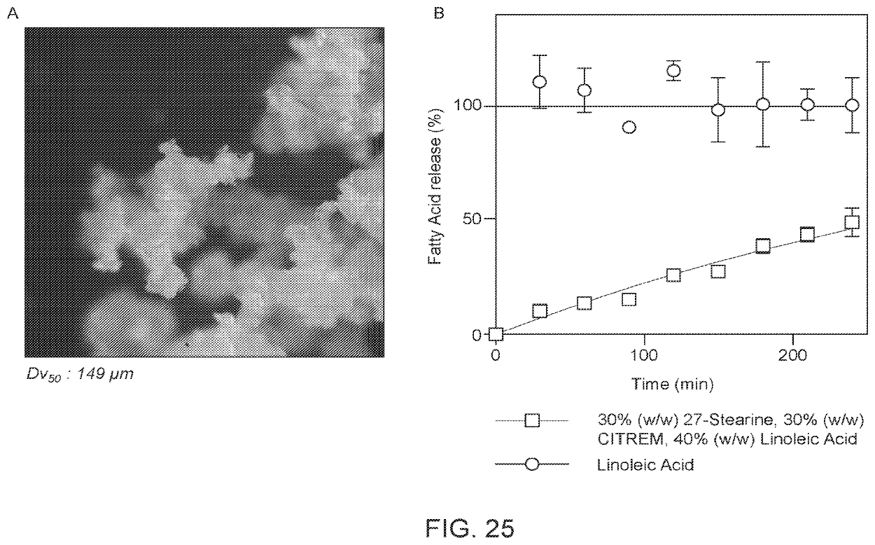

The following non-limiting example further demonstrates one or more core component(s) comprising one or more fatty acid(s) that were resistant to release and/or absorption in a bile salt-rich environment simulating digestive condition(s) of the gastrointestinal tract of one or more mammal(s). A mixture of 40% (w/w) linoleic acid, 30% (w/w) 27-Stearine, and 30% (w/w) CITREM was heated to 80° C. while stirring to allow for complete mixing, followed by cooling at 4° C. for 1 hour. The resulting solid mixture was cryo-milled at −192° C. with a 500 μm mesh filter; particle size analysis of the collected powder (FIG. 25A) indicated an average particle diameter, Dv50, of 149 μm. Six (6) 15 mL centrifuge tubes were filled with simulated intestinal fluid at 37° C. with 0.2% (w/v) sodium taurocholate. Formulated linoleic acid microparticles were added to 3 of these 6 tubes, while unformulated linoleic acid was added to the remaining 3 tubes. As shown in FIG. 25B, unformulated linoleic acid is rapidly emulsified in the bile-salt rich simulated intestinal fluid indicating rapid release in a simulated duodenum environment. In contrast, formulated linoleic acid resisted emulsification, with only 50% of loaded fatty acid released by 4 hours.

### BB. Example 28: Exemplary Core-Shell Preparation Exhibiting pH-Responsive Release of Carbohydrate(s)

In certain embodiments, one or more satiety modulating composition(s) are characterized as one or more of a core-shell preparation and/or particle preparation.

The following non-limiting example demonstrates the pH-responsive release of one or more carbohydrate(s) comprising one or more satiety modulating composition(s). In this example, one or more core-shell preparation(s) comprising a core further comprised of carbohydrate and multiple shells further comprising carbohydrate(s). One or more shell layer(s) was found to confer pH responsiveness towards said core-shell preparation(s).

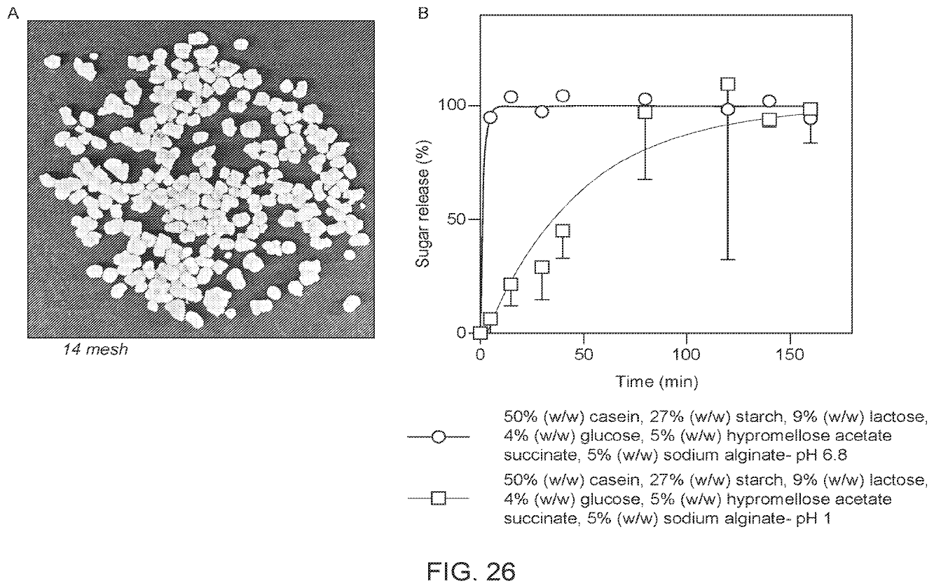

In this non-limiting example, a powdered mixture of 55% micellar casein, 30% directly compressible starch, 10% lactose, and 5% glucose was granulated using a Caleva Multi-Lab, followed by extrusion and spheronization via 1 mm×1 mm dies with constant addition of Dry-Flo starch. The resulting spherical particles were dried in an oven at 45° C. for 16 hours and passed through a 14-mesh sieve. Individual free particles were weighed and loaded into a VFC-LAB Micro FLO-COATER (Freund Vector) and sprayed with a 10% (w/v) hypromellose acetate succinate dispersion in water followed by a 2% (w/v) sodium alginate solution in water. A 5% coating weight gain was targeted for each layer, resulting in a total 10% weight gain coating for glucose-containing microparticles. The final concentration of all constituents in the particle preparation was 50% (w/w) casein, 27% (w/w) starch, 9% (w/w) lactose, 4% (w/w) glucose, 5% (w/w) hypromellose acetate succinate, and 5% (w/w) sodium alginate. Three (3) 15 mL centrifuge tubes were filled with simulated gastric fluid (SGF) at 37° C., with a pH of 1. Three (3) 15 mL centrifuge tubes were filled with simulated intestinal fluid (SIF) at 37° C., with a pH of 6.8. 180 mg of coated glucose-containing particles were added to each tube (FIG. 26A). As shown in FIG. 26B, the release of glucose from these particles was rapid in SIF, with complete release after only 5 minutes; in contrast, complete release of encapsulated glucose was delayed to 60 minutes of incubation in SGF.

### CC. Example 29: Exemplary Core-Shell Preparation Exhibiting pH-Responsive Release of Protein(s)

The following non-limiting example describes one or more core-shell preparation(s) comprising a core further comprised of protein and multiple shells further comprising carbohydrate(s). One or more shell layer(s) is found to confer pH responsiveness towards said core-shell preparation(s).

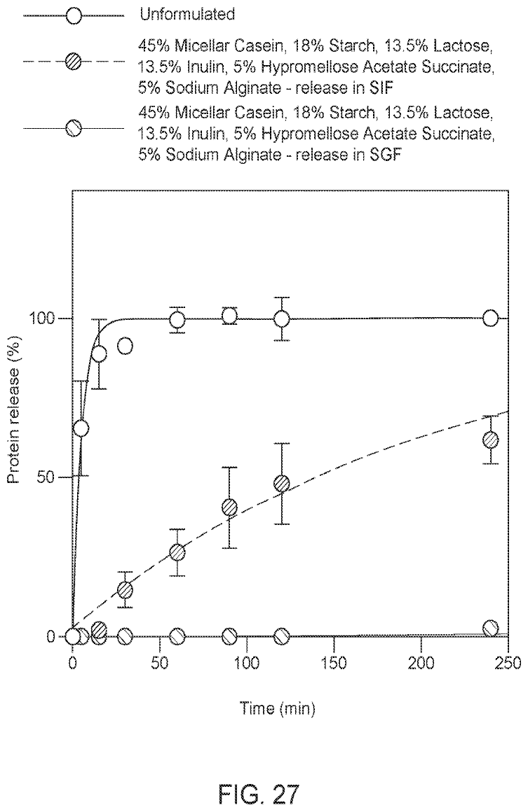

In this non-limiting example, a powdered mixture of 50% micellar casein, 20% directly compressible starch, 15% lactose, and 15% inulin was granulated using a Caleva Multi-Lab, followed by extrusion and spheronization via 1 mm×1 mm dies with constant addition of Dry-Flo starch. The resulting spherical particles were dried in an oven at 45° C. for 16 hours and passed through a 14-mesh sieve. Individual free particles were weighed and loaded into a VFC-LAB Micro FLO-COATER (Freund Vector) and sprayed with a 10% (w/v) hypromellose acetate succinate dispersion in water followed by a 2% (w/v) sodium alginate solution in water. A 5% coating weight gain was targeted for each layer, resulting in a total 10% weight gain. The final concentration of all constituents in the particle preparation was 45% (w/w) micellar casein, 18% (w/w) starch, 13.5% (w/w) lactose, 13.5% (w/w) inulin, 5% (w/w) hypromellose acetate succinate, and 5% (w/w) sodium alginate. Three (3) 15 mL centrifuge tubes were filled with simulated gastric fluid (SGF) at 37° C., with a pH of 1. Three (3) 15 mL centrifuge tubes were filled with simulated intestinal fluid (SIF) at 37° C., with a pH of 6.8. As shown in FIG. 27, the release of protein from these particles was steady in SIF, with 50% release after only 4 hours; in contrast, negligible release of encapsulated protein was observed following 4 hours of incubation in SGF.

### DD. Example 30: Exemplary Core-Shell Preparations Derived from Several Manufacturing Processes and Incorporation of Particle Preparations into Commercial Products

The following non-limiting example illustrates particle preparation(s) deriving from methods of manufacture of one or more satiety composition(s). One or more methods of manufacture of one or more satiety composition(s) is or may be selected to provide for desired characteristic(s) exhibited by particle preparation(s). For example, one or more methods of manufacture is or may be employed to generate particle(s) exhibiting one or more size distributions. In some cases, one or more size distribution(s) resulting from one or more methods of manufacture may be ascertained using microscopy. For example, one or more methods of manufacture is or may be employed to generate particle(s) exhibiting one or more size distributions. In some cases, one or more size distribution(s) resulting from one or more methods of manufacture may be ascertained using laser diffraction particle size analysis. Without wishing to be bound by any particular theory, it is contemplated that size distribution(s) exhibited by one or more particle preparation(s) are particularly advantageous for homogenous mixing within food and/or beverage product matrices. Without wishing to be bound by any particular theory, it is contemplated that size distribution(s) exhibited by one or more particle preparation(s) are particularly advantageous for minimizing sensory impact for consumers.

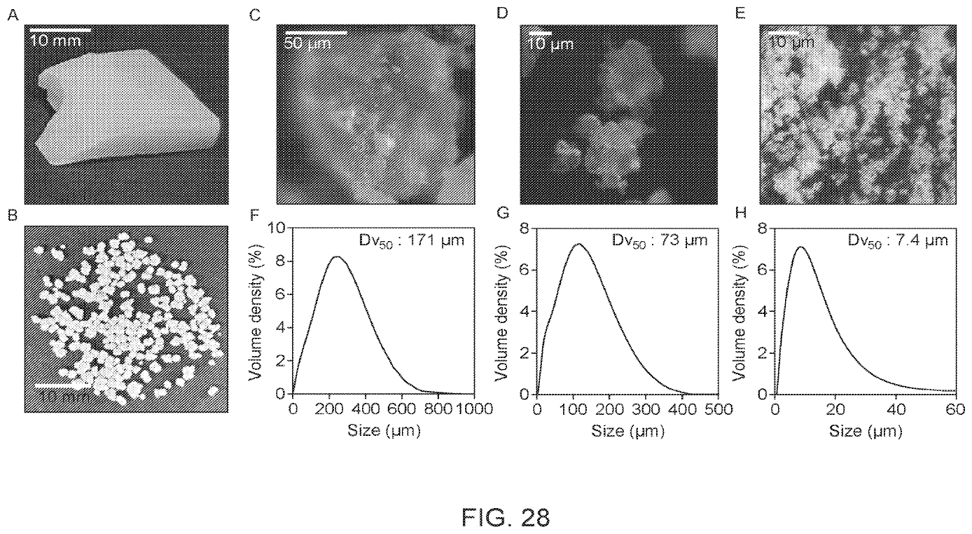

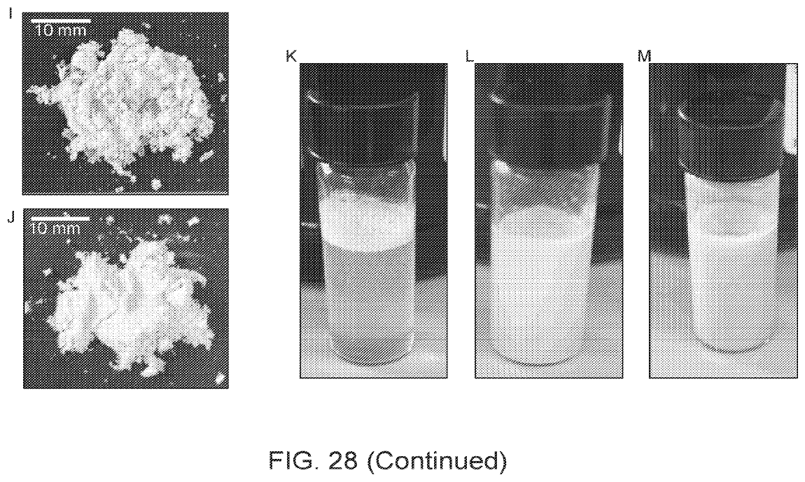

In this non-limiting example, several particle preparation(s) were generated using one or more methods of manufacture, with commensurate microscopy and particle size analysis data. Methods of manufacture comprising hot high shear homogenization of matrix component(s) and payload component(s) yielded solid “bars”, of which one non-limiting example is provided in FIG. 28A. The provided “bar” was macroscopic, with a diameter of 20 mm, and had a composition of 60% (w/w) oleic acid, 20% (w/w) ethyl cellulose 100 cP, and 20% (w/w) whey protein. Methods of manufacture comprising granulation, extrusion, and spheronization of matrix component(s) and payload component(s) yielded solid pellets, of which one non-limiting example is provided in FIG. 28B. The provided pellets were macroscopic, with a diameter of 1 mm, and had a composition of 50% (w/w) casein, 27% (w/w) starch, 9% (w/w) lactose, 4% (w/w) inulin, 5% (w/w) Hypromellose, and 5% (w/w) ethyl cellulose. Methods of manufacture comprising hot melt extrusion and hammer milling of matrix component(s) and payload component(s) yielded a coarse powder, of which one non-limiting example is provided in FIG. 28C. The provided pellets were microscopic, with an average diameter, Dv₅₀, of 171 μm, and had a composition of 50% (w/w) whey, 30% (w/w) 27 Stearine, and 20% (w/w) ethyl cellulose 100 cP. Methods of manufacture comprising hot melt extrusion and hammer milling of matrix component(s) and payload component(s) yielded a coarse powder, of which one non-limiting example is provided in FIG. 28D. The provided pellets were microscopic, with an average diameter, Dv₅₀, of 171 μm, and had a composition of 40% (w/w) whey, 40% (w/w) 27 Stearine, and 20% (w/w) calcium hydroxybutyrate. Methods of manufacture comprising spray drying of matrix component(s) and payload component(s) yielded a fine powder, of which one non-limiting example is provided in FIG. 28E. The provided powder was microscopic, with an average diameter, Dv₅₀, of 7.4 μm, and had a composition of 40% (w/w) whey, 40% (w/w) sodium alginate, and 20% (w/w) succinic acid. Incorporation of spheronized particles (FIG. 28B) into commercial whey protein powder (Muscle Milk, Pepsi) exhibits poor integration (FIG. 28I) in contrast to incorporation of spray dried particle preparation(s) (FIG. 28E) into commercial whey protein powder (Muscle Milk, Pepsi), illustrated in FIG. 28J, where no differences in texture or color were observed. Incorporation of milled preparation(s) (FIG. 28D) into liquids, illustrated in FIGS. 28K-28M, was challenging, leading to particle agglomeration at the liquid-air interface (FIG. 28K). Inclusion of 5% soybean lecithin improved mixing of formulation (FIG. 28L), and facilitated uniform incorporation into commercial enteral feed formula (Nutren® 1.0, Nestle) (FIG. 28M).

### EE. Example 31: Exemplary Eicosapentaenoic Acid Particle Preparations and Incorporation into Yogurt

The following non-limiting example illustrates particle preparations including a liquid satiety modulator, a lipid, a protein, a carbohydrate, and/or an acaloric phytonutrient, and the incorporation of said particle preparations into yogurt.

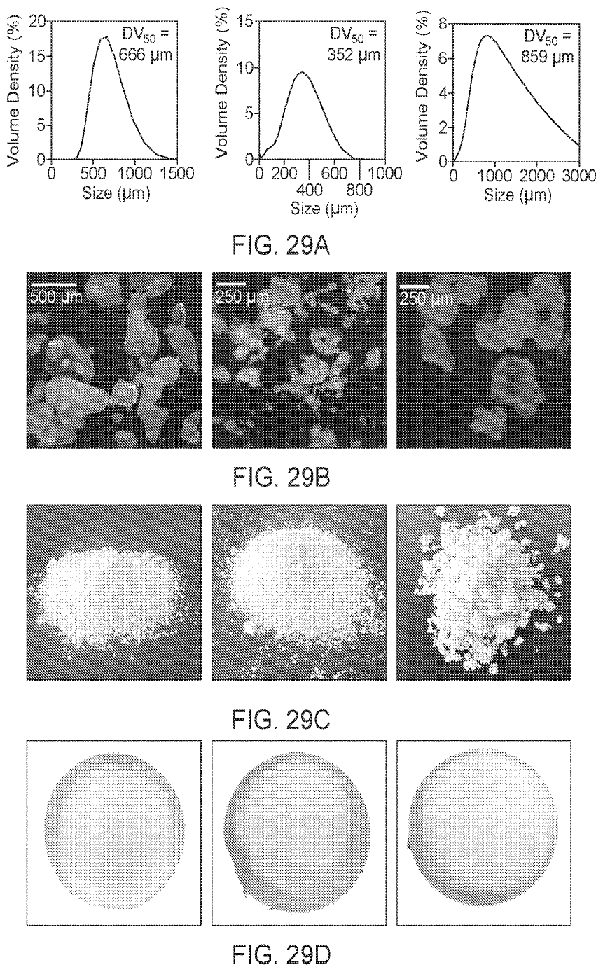

To prepare a first exemplary particle preparation shown in FIG. 29A (Left Panel), FIG. 29B (Left Panel), and FIG. 29C (Left Panel), carnauba wax, stearic acid, α-tocopherol, and ethyl cellulose (100 cP) were dispersed together at 180° C. The mixture was periodically stirred at 50RPM to homogenize contents. Eicosapentaenoic acid (Epax) was added and the complete mixture was stirred at 150 RPM for 5 minutes. The formulation was allowed to cool overnight at 4° C. prior to further processing. The completed formulation was segmented into 3 cm² chunks and chilled in liquid nitrogen (−198° C.). The particle formulation/liquid nitrogen mixture was passed through an IKA MF 10 cutting grinding mill at 3000 RPM equipped with a 1 mm filter. As sample was passed through the mill, liquid nitrogen was continuously poured into the intake to ensure that the particle preparation remained brittle. Collected particle preparations were stored at room temperature until further use. The resulting particle preparation included 50% (w/w) eicosapentaenoic acid, 15% (w/w) carnauba wax, 15% (w/w) stearic acid, 15% (w/w) ethyl cellulose 100 cP, and 5% (w/w) α-tocopherol. The particle preparation was a light yellow powder (FIG. 29C; Left Panel) with an average particle size (Dv50) of 666 μm (FIG. 29A; Left Panel and FIG. 29B; Left Panel). As shown in FIG. 29D (Left Panel), the particle preparation incorporated well into a commercial dairy product (Chobani vanilla yogurt).

To prepare a second exemplary particle preparation shown in FIG. 29A (Middle Panel), FIG. 29B (Middle Panel), and FIG. 29C (Middle Panel), EUDRAGUARD© Protect (EPO) and calcium carbonate were mixed and briefly passed through a coffee grinder to homogenize particle size. Eicosapentaenoic acid and α-tocopherol were added to the dry powders and mixed manually until homogenous. The mixture was then extruded in a Haake MiniLab 3 instrument at 55° C., 25RPM. The extruded formulation was immersed in liquid nitrogen then passed through an IKA MF 10 cutting grinding mill at 3000 RPM equipped with a 1 mm filter. As sample was passed through the mill, liquid nitrogen was continuously poured into the intake to ensure that the particle preparation remained brittle. Collected particle preparations were stored at room temperature until further use. The resulting particle preparation included 25% (w/w) eicosapentaenoic acid, 50% (w/w) EUDRAGUARD Protect, 15% (w/w) calcium carbonate, and 10% (w/w) α-tocopherol. The particle preparation was a white powder (FIG. 29C; Middle Panel) with an average particle size (Dv50) of 325 μm (FIG. 29A; Middle Panel and FIG. 29B; Middle Panel). As shown in FIG. 29D (Middle Panel), the particle preparation incorporated well into a commercial dairy product (Chobani vanilla yogurt).

To prepare a third exemplary particle preparation shown in FIG. 29A (Right Panel), FIG. 29B (Right Panel), and FIG. 29C (Right Panel), 100 mg/mL of whey protein isolate solution was prepared and pH adjusted to 7.5 with sodium hydroxide. A mixture of sesame oil, sitosterol, diindolylmethane, α-tocopherol, and eicosapentaenoic acid was added to the solution. This mixture was allowed to homogenize for an hour at 50° C. and stirred at 150 RPM. Once homogenous, the mixture was placed in a water bath set to 95° C. for one hour. To ensure homogeneity the denatured gel was mixed once at t=30 mins, spread on a baking tray, and placed into a 60° C. drying oven overnight (approximately 16 hours). The complete formulation was then chilled in liquid nitrogen (−198° C.). The particle formulation/liquid nitrogen mixture was passed through an IKA MF 10 cutting grinding mill at 3000 RPM equipped with a 3 mm filter. As sample was passed through the mill, liquid nitrogen was continuously poured into the intake to ensure that the particle preparation remained brittle. Collected particle preparations were stored at room temperature until further use. The resulting particle preparation included 40% (w/w) eicosapentaenoic acid, 37% (w/w) whey protein isolate, 10% (w/w) sesame oil, 5% (w/w) diindolylmethane, 5% (w/w) sitosterol, 3% (w/w) α-tocopherol. The particle preparation was a light yellow powder (FIG. 29C; Right Panel) with an average particle size (Dv50) of 859 μm (FIG. 29A; Right Panel and FIG. 29B; Right Panel). As shown in FIG. 29D (Right Panel), the particle preparation incorporated well into a commercial dairy product (Chobani vanilla yogurt).

Thus, the data of the present example demonstrate that exemplary preparations of the present disclosure result in particles incorporating high concentrations of liquid satiety modulator (e.g., eicosapentaenoic acid) that are well-incorporated into commercial product matrices (e.g., yogurt).

### FF. Example 32: Enhanced Basolateral Membrane Permeability of Satiety Modulator

The following non-limiting example illustrates the enhanced permeability of a satiety modulator (e.g., eicosapentaenoic acid; EPA) across an intestinal epithelial cell monolayer when formulated in a particle preparation of the present disclosure.

Caco-2 cells were cultured then seeded onto multi-well transwell plates. Cells were maintained in Dulbecco's modified Eagle's Medium (DMEM) supplemented with 20% fetal bovine serum (FBS), for 21 days prior to dosing. The apical and basolateral transwell chambers were replaced with Hank's Balanced Salt Solution (HBSS) for conditioning and measurement of transepithelial electrical resistance (TEER). The apical chamber buffer was replaced with HBSS containing unformulated eicosapentaenoic acid (0.1% w/v EPA, 99.9% w/v water), or an emulsified mixture of 9 parts buffer with 1 part of an EPA preconcentrate having 10% (v/v) eicosapentaenoic acid, 40% (v/v) triacetin, and 50% (v/v) Tween 80. The final composition of unformulated EPA was 0.1% (w/v) EPA with 99.9% (w/v) water and the final composition of exemplary EPA formulation was 0.1% (w/v) EPA, 0.9% (w/v) ethanol, 4% (w/v) triacetin, 5% (w/v) Tween 80, and 90% (w/v) water. The contents of the basolateral chamber were collected at 0.5, 2, and 4 hours post-dosing and stored in polypropylene 96-well plates. The concentration of eicosapentaenoic acid in the basolateral chamber samples was measured using a standard Acyl-CoA synthetase-based fluorometric assay.

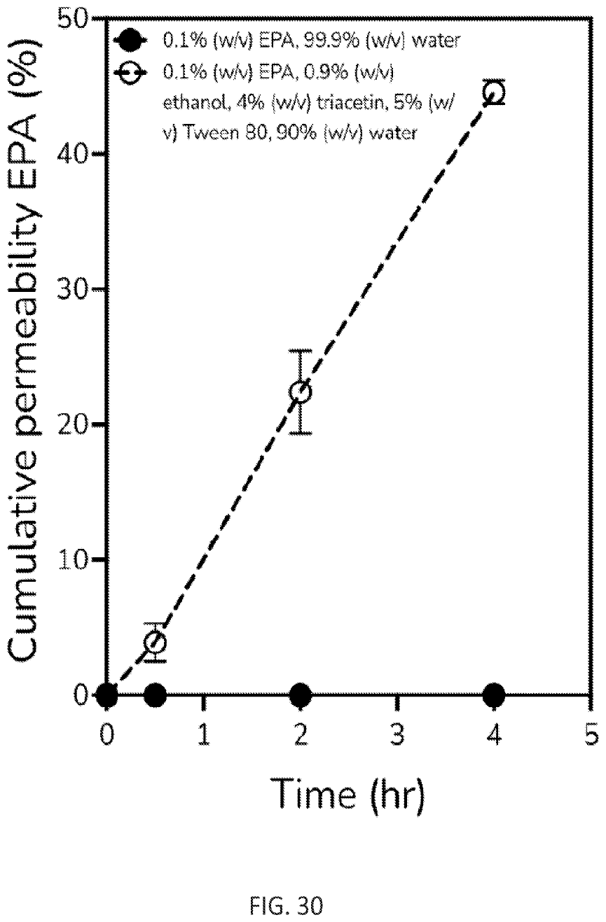

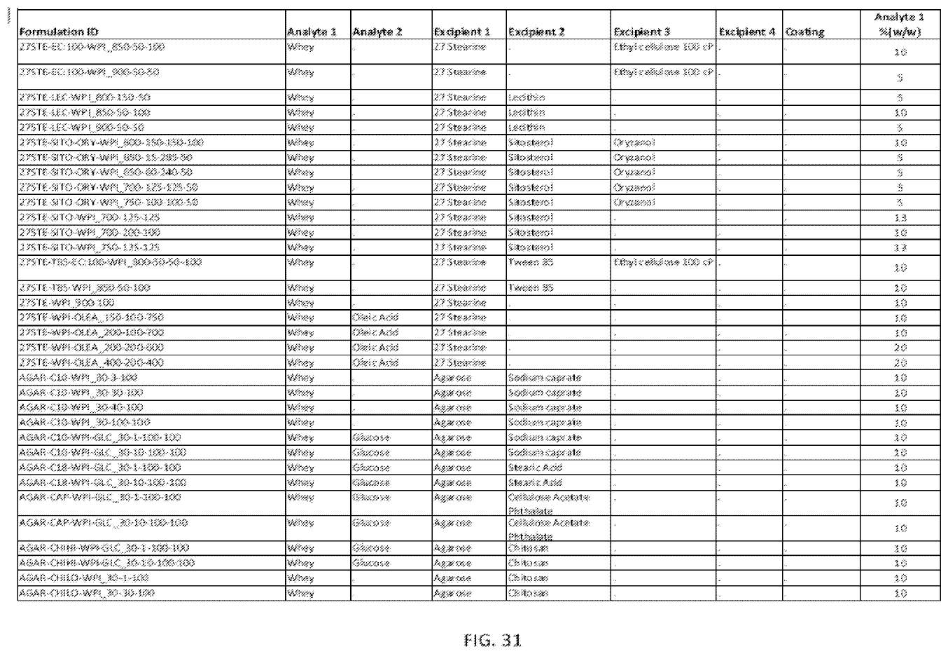

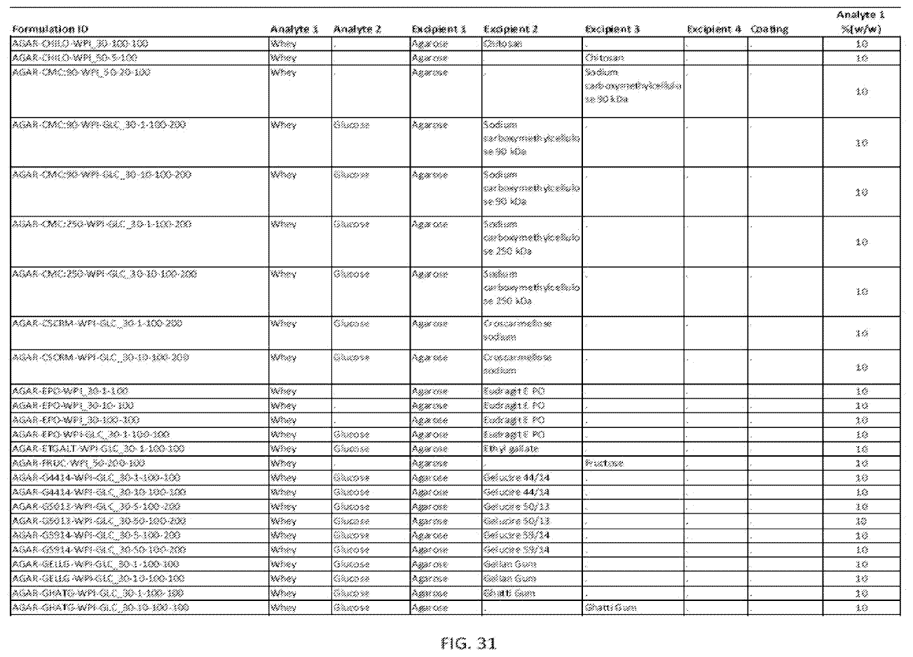

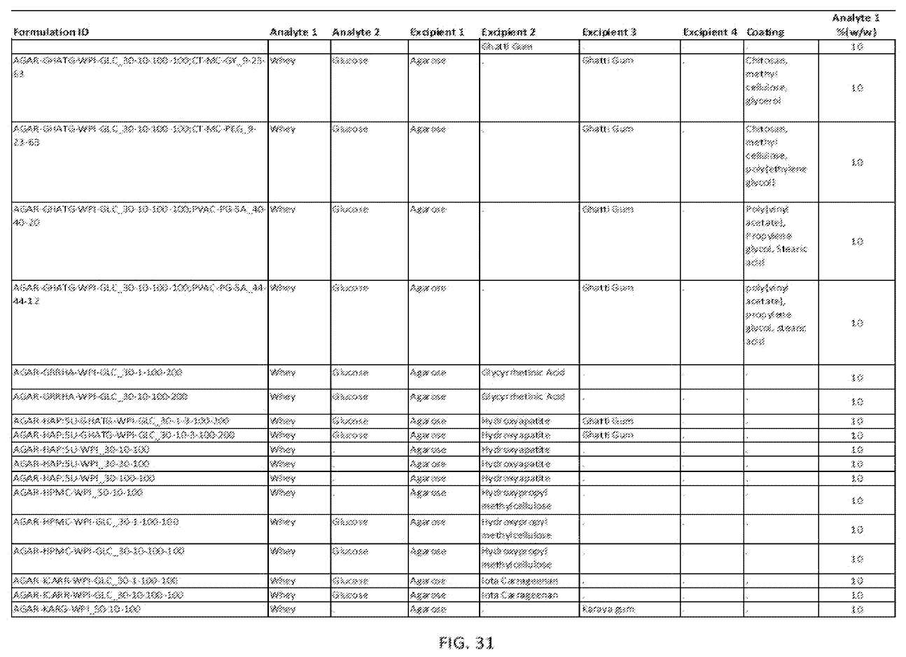

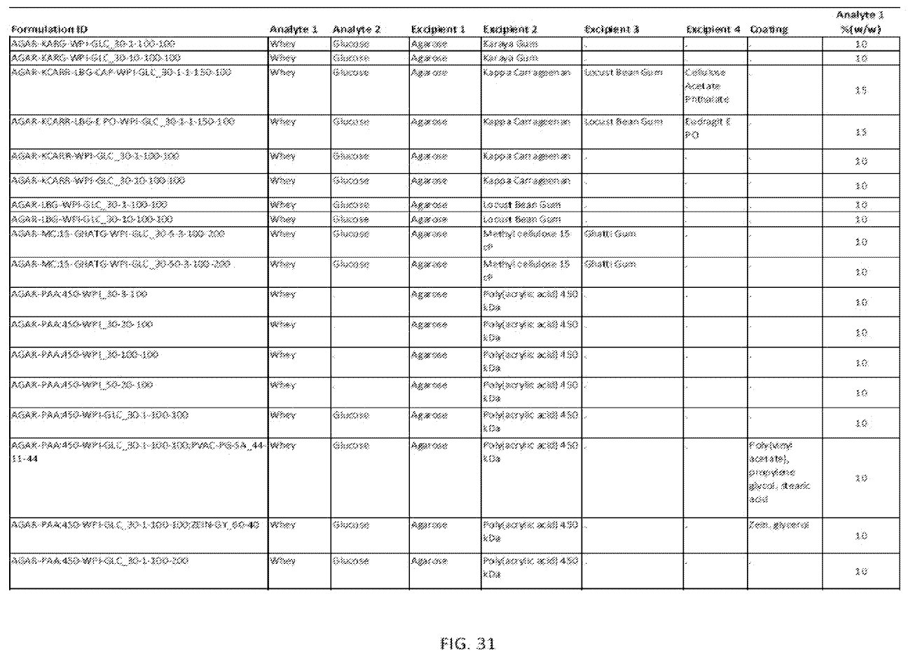

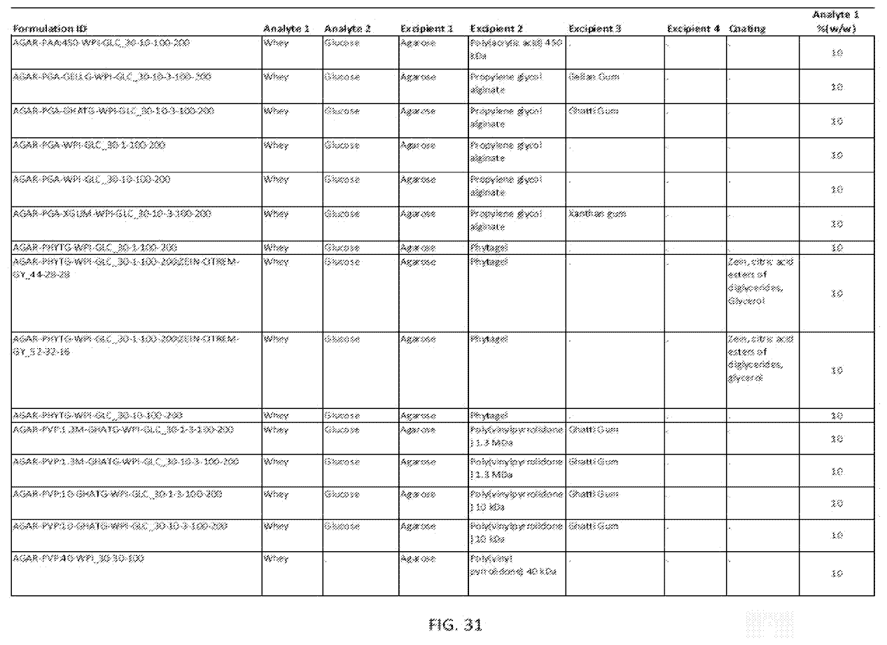

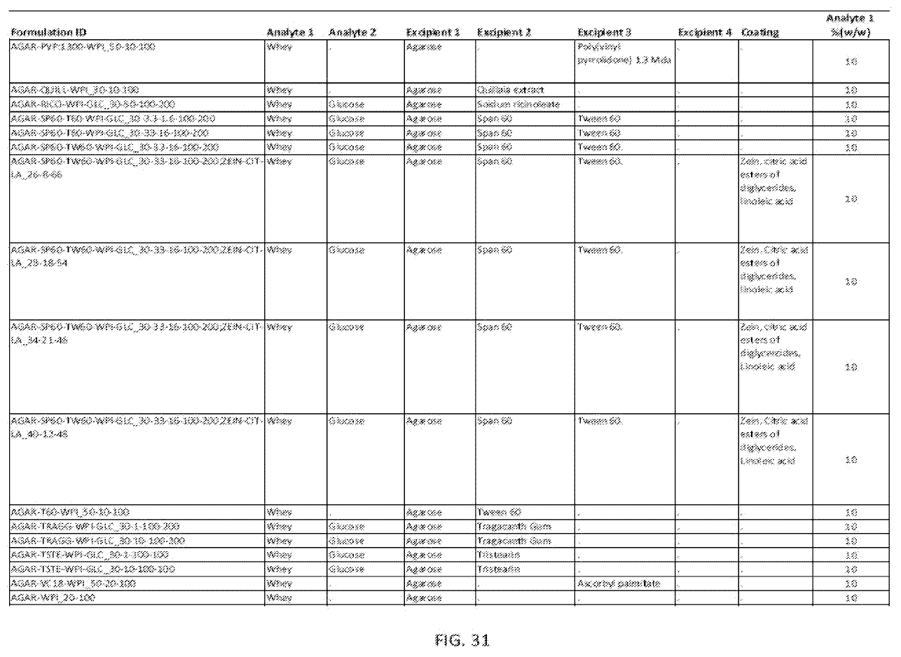

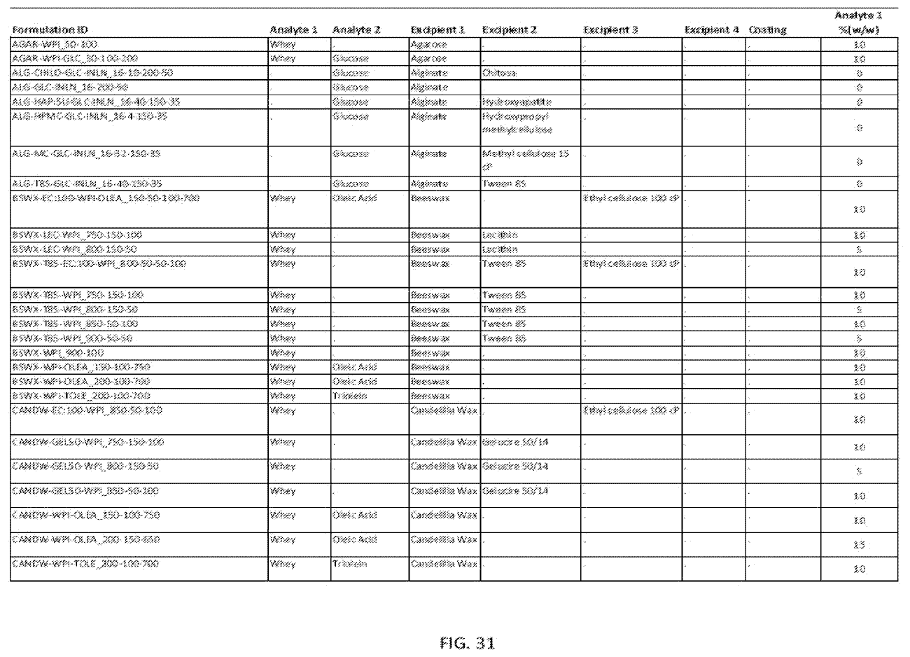

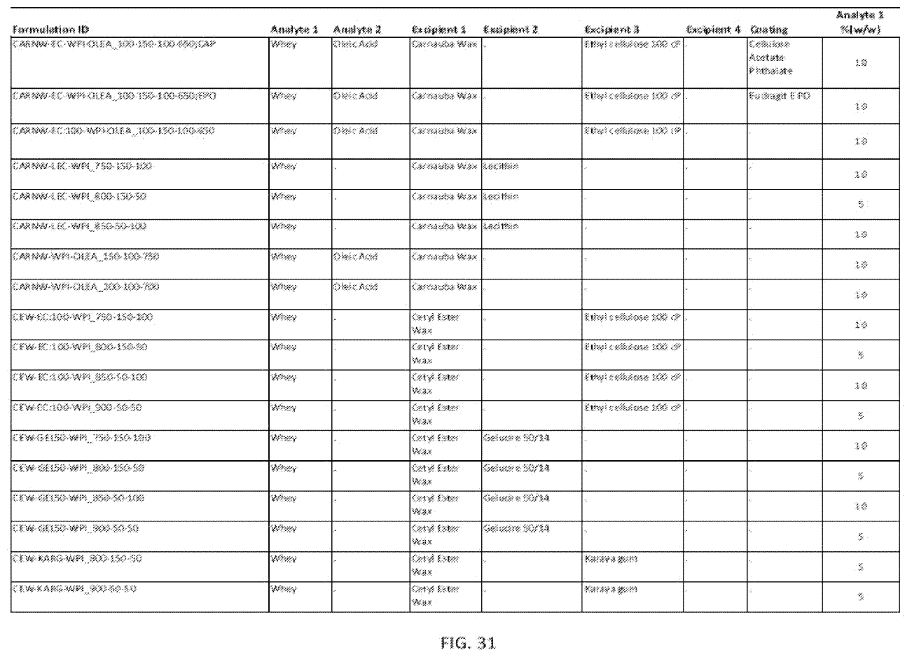

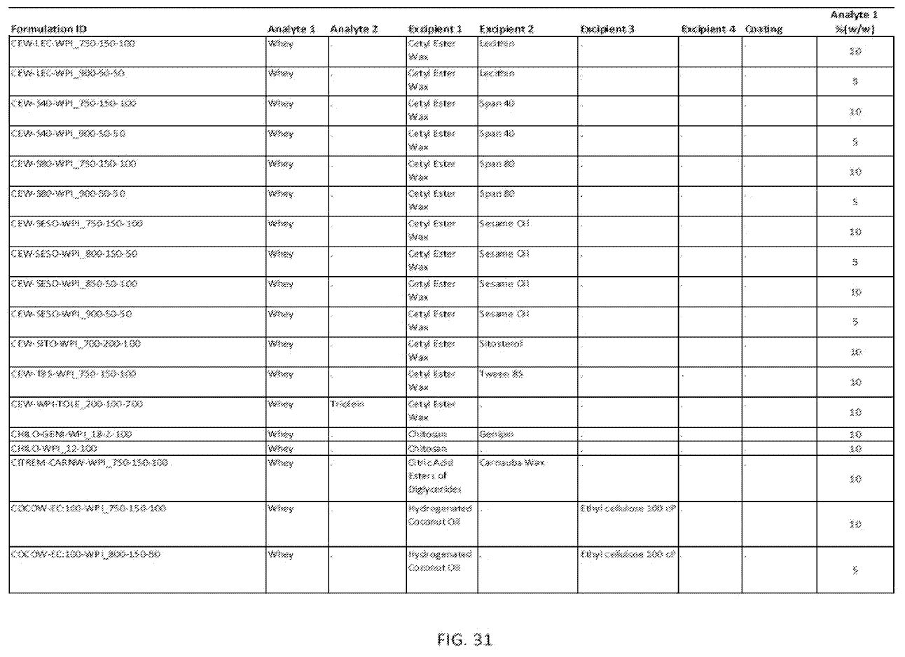

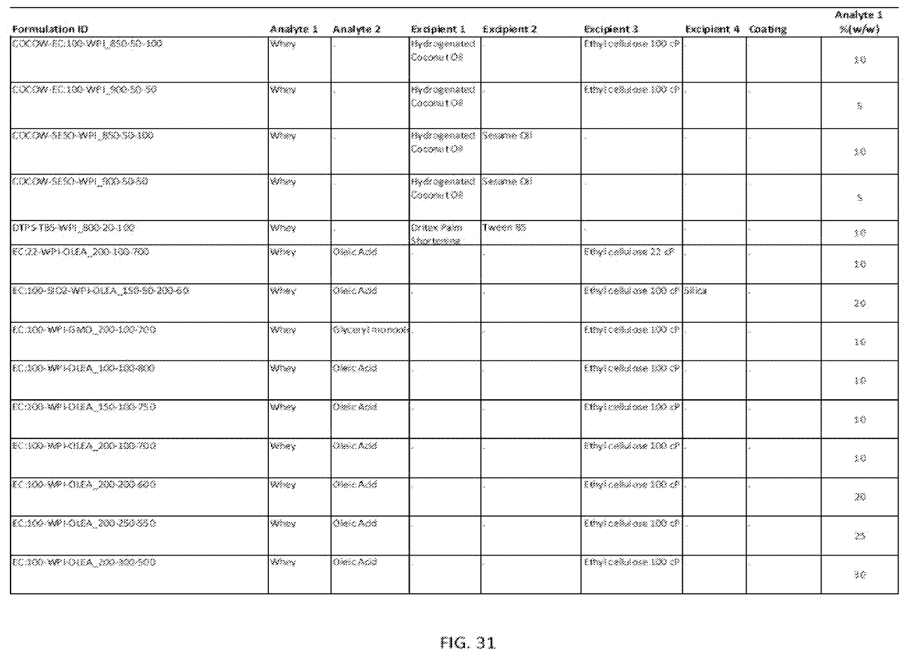

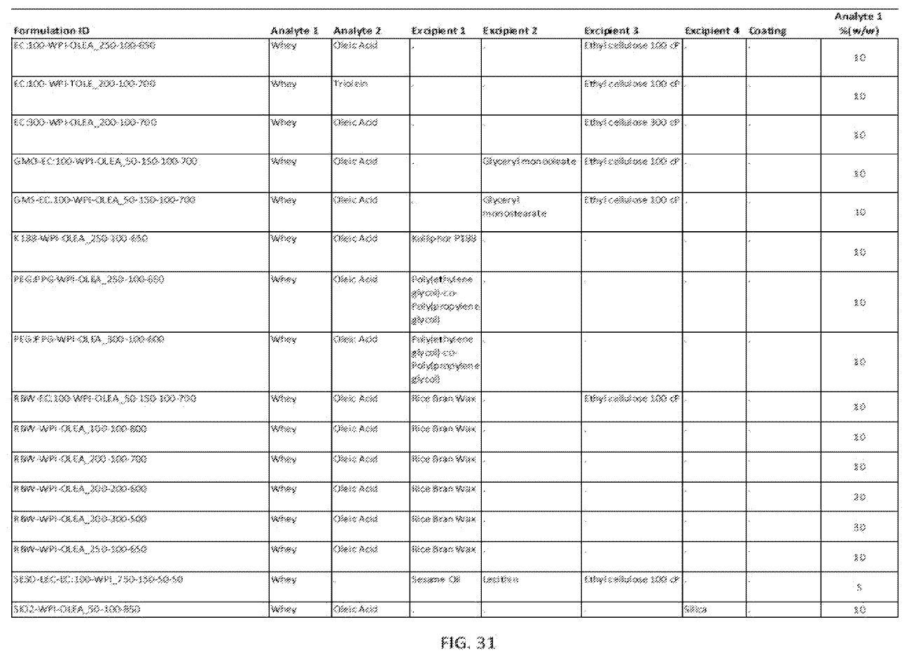

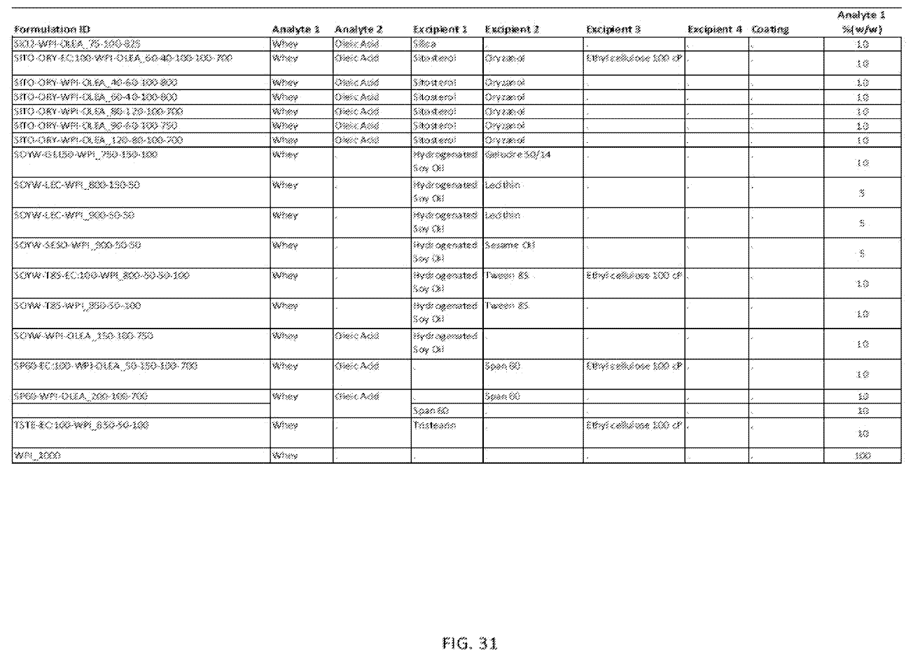

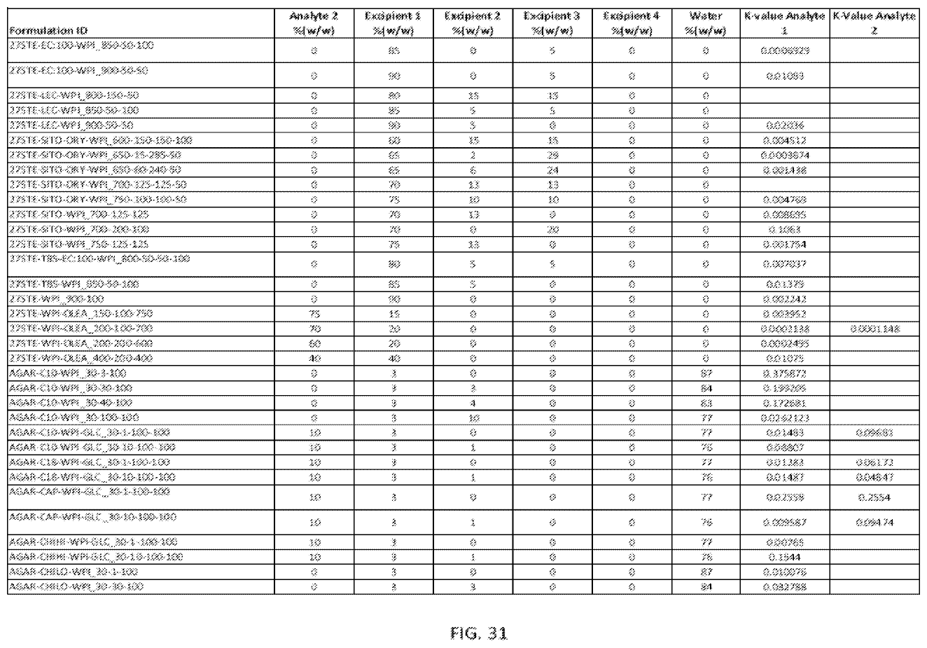

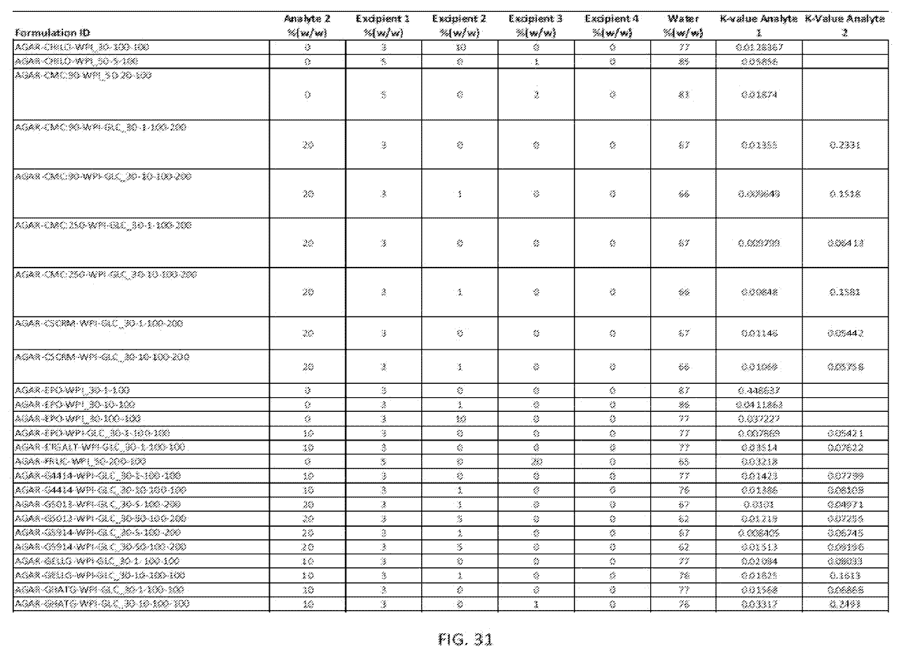

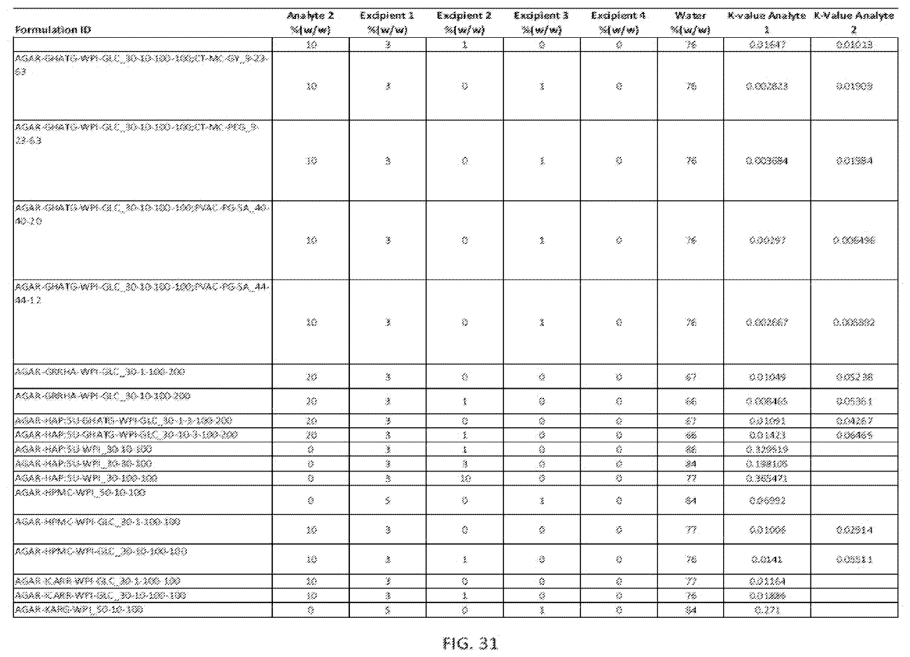

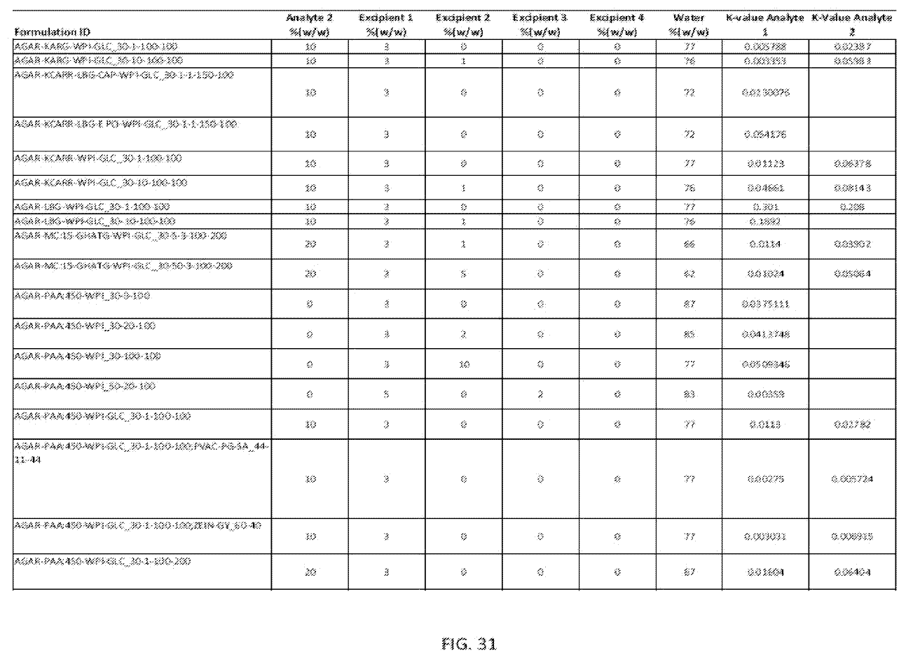

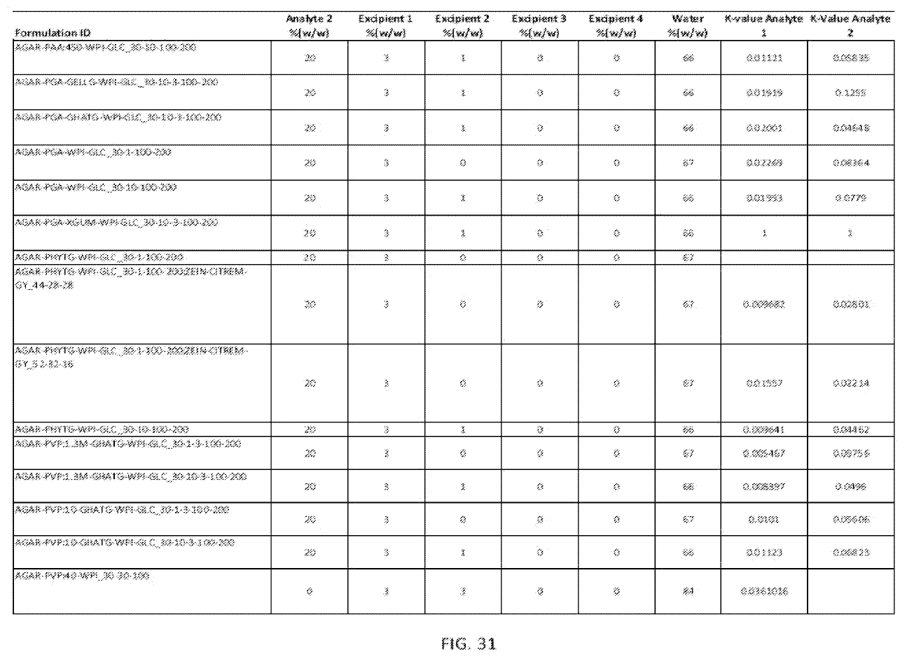

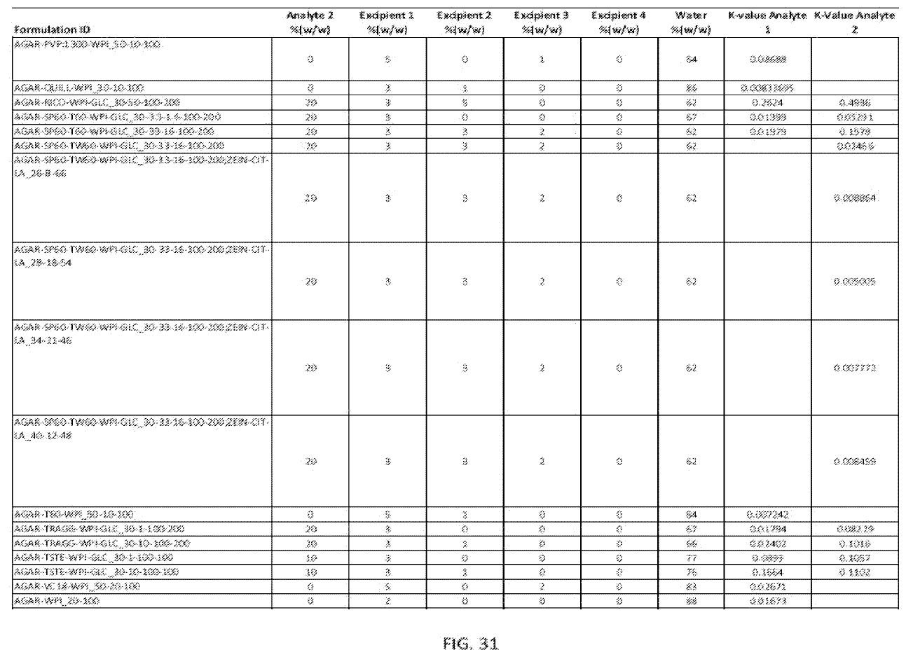

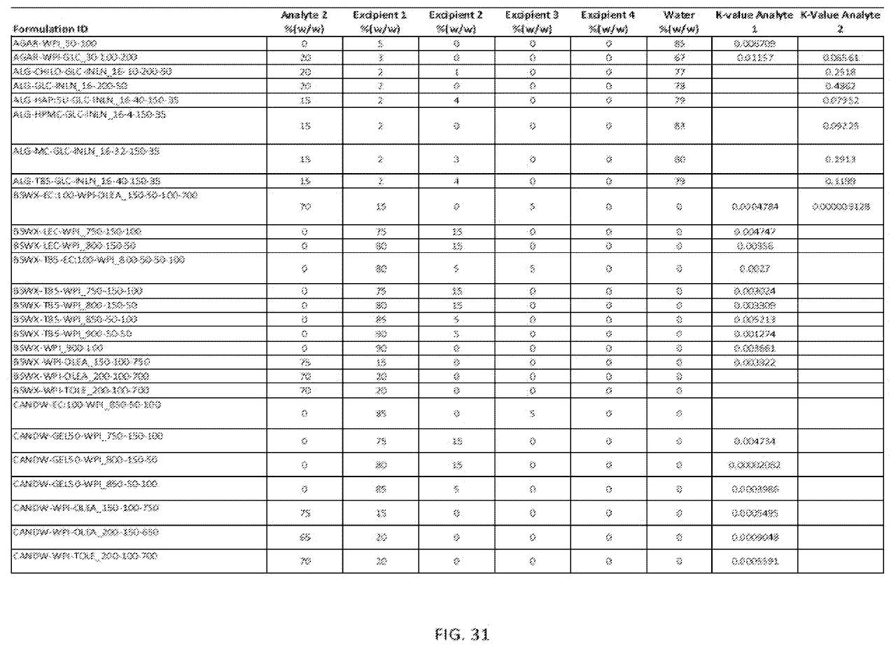

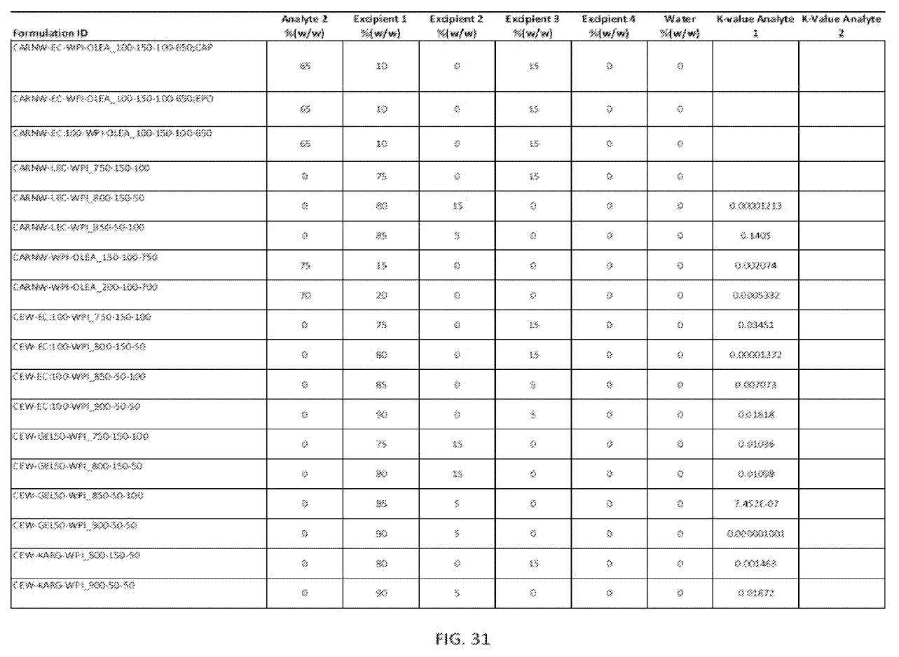

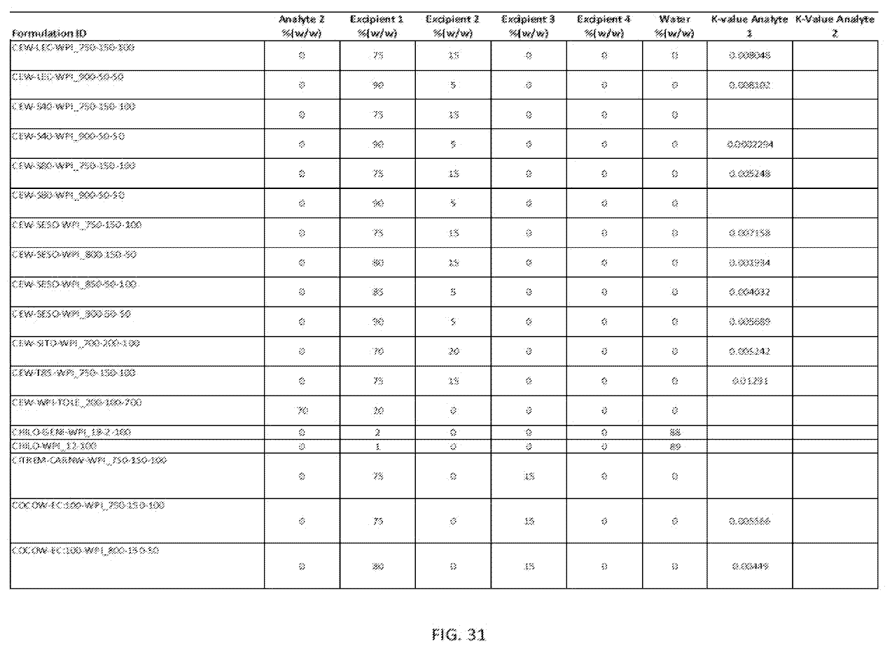

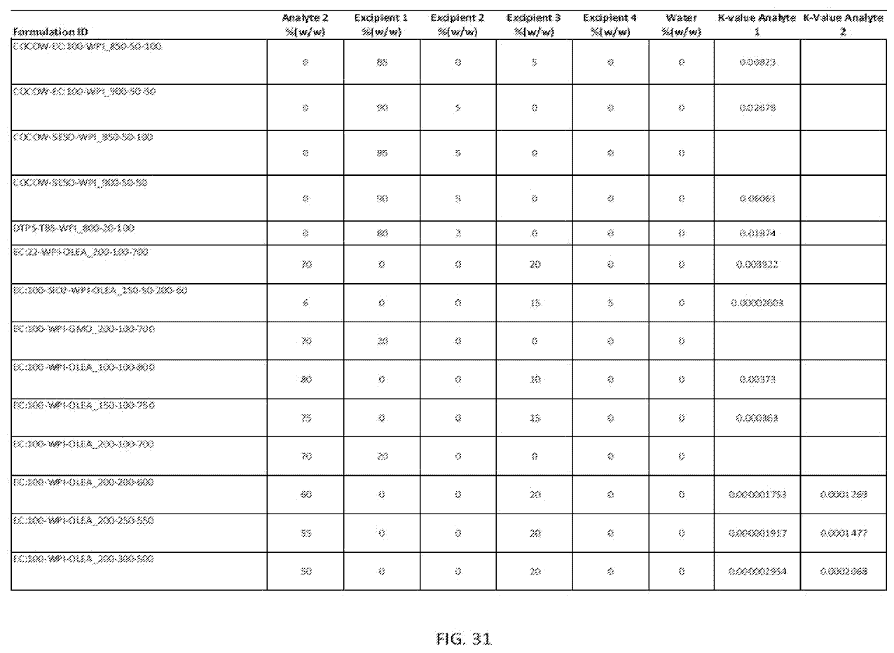

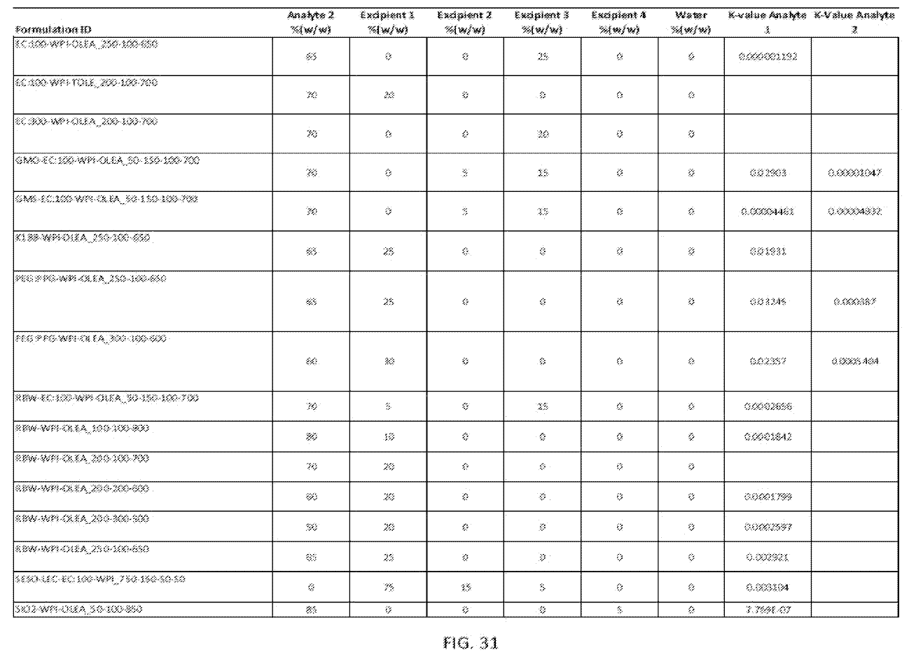

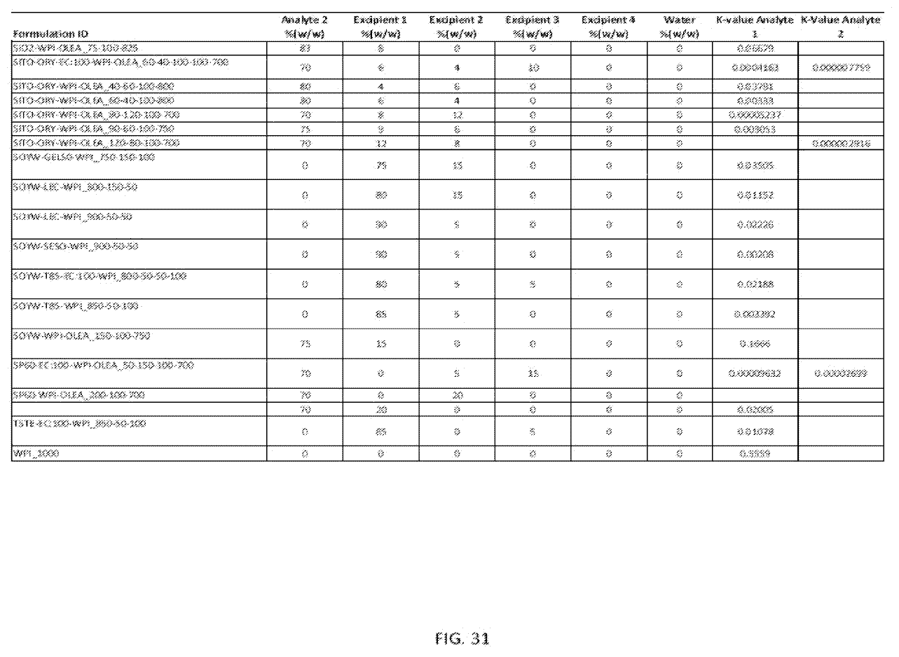

As shown in FIG. 30, an exemplary eicosapentaenoic acid particle preparation of the present disclosure (open circles) enhanced permeability/transport of EPA across the intestinal cell monolayer as compared to unformulated EPA.

These data indicate that exemplary particle formulations of the present disclosure are capable of enhancing bioavailability of satiety modulators when consumed by a subject.

What is claimed is:

1. A non-therapeutic particle preparation comprising a spatial arrangement of:
about 40% to about 75%, on a dry weight basis, of one or more lipid(s),
about 10% to about 30%, on a dry weight basis, of one or more protein(s),
about 10% to about 30%, on a dry weight basis, of one or more carbohydrate(s), and
about 5% to about 15%, on a dry weight basis, of one or more acaloric phytonutrient(s),
wherein at least 90% of the one or more lipid(s), the one or more protein(s), the one or more carbohydrate(s), and/or the one or more acaloric phytonutrient(s) are released in an upper gastrointestinal region, a lower gastrointestinal region, or any combination thereof of one or more mammal(s).

2. The composition of claim 1, wherein the particle preparation comprises at least 1, at least 10, at least 100, at least 1000, at least 10000, at least 100000, and/or at least 1000000 particles.

3. The composition of claim 1, wherein the particle preparation comprises at least 1, at least 10, at least 100, at least 1000, at least 10000, at least 100000, and/or at least 1000000 particles, wherein the particle preparation comprises different spatial arrangements of the one or more lipid(s), one or more protein(s), one or more carbohydrate(s), and/or one or more acaloric phytonutrient(s).

4. The composition of claim 1, wherein the one or more lipid(s) comprises a fatty acid and/or a fatty amide comprising all-cis α-linolenic acid, γ-linolenic acid, anandamide, arachidonic acid, adrenic acid, calendic acid, clupanodonic acid, docosadienoic acid, docosapentaenoic acid, docosahexaenoic acid, eicosadienoic acid, eicosapentaenoic acid, eicosatrienoic acid, elaidic acid, erucic acid, gondoic acid, linoleamide, linoleic acid, linolenylamide, mead acid, N-docosahexaenoylethanolamine, N-docosatetraenoylethanolamine, N-oleoylethanolamine, N-palmitoylethanolamine, N-stearoylethanolamine, nervonic acid, nisinic acid, oleamide, oleic acid, osbond acid, palmitoleic acid, pinolenic acid, paullinic acid, rumenic acid, stearidonic acid, tetracosapentaenoic acid, and/or vaccenic acid, and
wherein the one or more acaloric phytonutrient(s) comprises quercetin, kaempferol, myricetin, fisetin, rutin, isorhamnetin, naringenin, silybin, eriodictyol, apigenin, chrysin, delphinidin, betanin, cyanidin chloride, neohesperidin, epigallocatechin, diosmetin, baicalein, genistein, oleuropein, amarogentin, genipin, aucubin, catalpol, olivetol, cannabidiol, tetrahydrocannabinol, daidzein, pelargonidin, tangeritin, luteolin, wogonin, epicatechin, catechin, theaflavin, resveratrol, and/or hydroxytyrosol.

5. The composition of claim 1, wherein more than about 40% of the one or more lipid(s), protein(s), carbohydrate(s), and/or acaloric phytonutrient(s) is observed in the upper gastrointestinal tract of one or more mammal(s), and
wherein more than about 50% of the one or more lipid(s), protein(s), carbohydrate(s), and/or acaloric phytonutrient(s) is observed in the lower gastrointestinal tract of the one or more mammal(s).

6. The composition of claim 1, wherein the average diameter of the particle preparation is at least about 1 μm, at least about 10 μm, and/or at least about 20 μm as determined by laser diffractometry, and
wherein the average diameter of the particle preparation is no more than about 50 μm as determined by laser diffractometry.

7. The composition of claim 1, wherein the particle preparation comprises a core-shell structure of at least one core and at least two shells.

8. The composition of claim 7, wherein the particle preparation comprises a mononuclear core-shell structure.

9. The composition of claim 7, wherein the particle preparation comprises a polynuclear core-shell structure.

10. The composition of claim 7, wherein the at least one core and at least two shells are solid.

11. The composition of claim 10, wherein the at least one core and at least two shells each possess a melting point greater than about 25° C., greater than about 30° C., greater than about 40° C., greater than about 45° C., greater than about 50° C., greater than about 55° C., and/or greater than about 60° C.

12. The composition of claim 11, wherein the at least one core and/or at least two shells are responsive to at least one of: pH, temperature, time, light, water, living organisms, enzymes, bile salt, salt concentration, and mechanical forces.

13. The composition of claim 12, wherein at least one of the at least two shells is responsive to a pH less than about 4.5.

14. The composition of claim 12, wherein the at least one core and at least one of the at least two shells are responsive to a pH greater than about 6.8.

15. The composition of claim 12, wherein the at least one core and at least one of the at least two shells responsive to a pH greater than about 6.8 are completely encapsulated in at least one shell responsive to a pH less than about 4.5.

16. The composition of claim 12, wherein the one or more lipid(s), one or more protein(s), one or more carbohydrate(s), and/or one or more acaloric phytonutrient(s) impart responsiveness to the at least one core and/or at least two shells.

17. The composition of claim 12, wherein the at least one core comprises one or more lipid(s), one or more protein(s), one or more carbohydrate(s), and/or one or more acaloric phytonutrient(s).

18. The composition of claim 12, wherein the at least two shells comprise one or more lipid(s), one or more protein(s), one or more carbohydrate(s), and/or one or more acaloric phytonutrient(s).

19. The composition of claim 1, wherein the one or more lipid(s), one or more protein(s), one or more carbohydrate(s), and/or one or more acaloric phytonutrient(s) together comprise 100% of the dry weight.

20. The composition of claim 19, wherein the one or more lipid(s), one or more protein(s), one or more carbohydrate(s), and/or one or more acaloric phytonutrient(s) are characterized as safe for consumption by one or more mammal(s).

21. The composition of claim 20, wherein the one or more lipid(s), one or more protein(s), one or more carbohydrate(s), and/or one or more acaloric phytonutrient(s) are characterized as safe for consumption by human(s).

22. The composition of claim 21, wherein the one or more lipid(s), one or more protein(s), one or more carbohydrate(s), and/or one or more acaloric phytonutrient(s) are characterized as edible.

23. The composition of claim 22, wherein the one or more lipid(s) comprises at least one component selected from the group consisting of fatty acids, fatty amides, sterols, terpenes, and terpenoids.

24. The composition of claim 23, wherein the one or more lipid(s) characterized as a fatty acid and/or fatty amide is unsaturated.

25. The composition of claim 24, wherein the one or more lipid(s) characterized as unsaturated is further characterized as all-cis.

26. The composition of claim 25, wherein the one or more fatty acid(s) and/or fatty amide(s) possesses a carbon chain length between 10 and 26.

27. The composition of claim 26, wherein the fatty amide is an amide of ammonia, ethylamine, and/or ethanolamine.

28. The composition of claim 27, wherein the one or more lipid(s) characterized as a fatty acid and/or fatty amide is all-cis α-linolenic acid, γ-linolenic acid, anandamide, arachidonic acid, adrenic acid, calendic acid, clupanodonic acid, docosadienoic acid, docosapentaenoic acid, docosahexaenoic acid, eicosadienoic acid, eicosapentaenoic acid, eicosatrienoic acid, elaidic acid, erucic acid, gondoic acid, linoleamide, linoleic acid, linolenylamide, mead acid, N-docosahexaenoylethanolamine, N-docosatetraenoylethanolamine, N-oleoylethanolamine, N-palmitoylethanolamine, N-stearoylethanolamine, nervonic acid, nisinic acid, oleamide, oleic acid, osbond acid, palmitoleic acid, pinolenic acid, paullinic acid, rumenic acid, stearidonic acid, tetracosapentaenoic acid, and/or vaccenic acid.

29. The composition of claim 28, wherein the one or more lipid(s) characterized as a fatty acid and/or fatty amide is esterified and/or amidated to a glycerol backbone as a monoglyceride, a diglyceride, and/or a triglyceride.

30. The composition of claim 28, wherein the one or more lipid(s) characterized as a fatty acid and/or fatty amide is esterified, and/or amidated to ethylene glycol, propylene glycol, sucrose, maltose, xylitol, erythritol, sorbitol, and/or mannitol.

31. The composition of claim 23, wherein the one or more lipid(s) characterized as a sterol is γ-oryzanol, abietane, abietic acid, brassicasterol, campestanol, campesterol, cholestanol, cholesterol, ergosterol, sitostanol, sitosterol, oleanolic acid, ursolic acid, betulinic acid, moronic acid, cafestol, limonene, hinokitiol, carvone, menthol, linalool, thujene, and/or stigmasterol.

32. The composition of claim 22, wherein the one or more protein(s) is a nutritional protein, a structural protein, an enzyme, a polypeptide, an oligopeptide, or an amino acid.

33. The composition of claim 32, wherein the one or more protein(s) is calcium caseinate, corn protein isolate, milk protein concentrate, oat protein isolate, pea protein isolate, sodium caseinate, soy protein isolate, wheat protein isolate, and/or whey protein isolate.

34. The composition of claim 32, wherein the one or more protein(s) is avenin, collagen, fibroin, gelatin, glutanin, kefirin, and/or zein.

35. The composition of claim 32, wherein the one or more protein(s) is an amylase, a cellulase, a lipase, and/or a protease.

36. The composition of claim 32, wherein the one or more protein(s) is amylin, cholecystokinin, GIP, GLP-1, GLP-2, oxyntomodulin, peptide YY, and/or somatostatin.

37. The composition of claim 32, wherein the one or more protein(s) is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine.

38. The composition of any one of claims 33-37, wherein the one or more protein(s) is characterized as unmodified, denatured, hydrolyzed, and/or complexed.

39. The composition of claim 22, wherein the one or more carbohydrate(s) is a monosaccharide, a disaccharide, and/or a polysaccharide.

40. The composition of claim 39, wherein the one or more carbohydrate(s) is extracted from natural sources.

41. The composition of claim 39, wherein the one or more carbohydrate(s) is characterized as fully synthetic.

42. The composition of claim 39, wherein the one or more carbohydrate(s) is characterized as semi-synthetic.

43. The composition of claim 40, wherein the one or more carbohydrate(s) extracted from natural sources is chemically modified.

44. The composition of claim 39, wherein the one or more carbohydrate(s) is a nutrient.

45. The composition of claim 44, wherein the one or more carbohydrate(s) is digestible.

46. The composition of claim 45, wherein the one or more carbohydrate(s) is a source of energy.

47. The composition of claim 46, wherein the one or more carbohydrate(s) is D-glucose, D-ribose, D-arabinose, D-allose, D-galactose, D-fructose, D-psicose, D-sucrose, D-tagatose, D-isomaltulose, D-lactose, D-maltose, D-trehalose, D-inositol.

48. The composition of claim 44, wherein the one or more carbohydrate(s) is a source of dietary fiber.

49. The composition of claim 48, wherein the one or more carbohydrate(s) is amylopectin, dextran, pectin, amylose, inulin, locust bean gum, maltodextrin, xanthan gum, gum arabic, karaya gum, ghatti gum, guar gum, sodium carboxymethylcellulose, sodium alginate, sodium hyaluronate, calcium alginate, agarose, chitosan, chitin, carrageenan, chondroitin sulfate, hydroxypropyl methylcellulose, methyl cellulose, modified cellulose gum, ethyl cellulose, hydroxyethylcellulose, corn starch, cellulose triacetate, cellulose acetate butyrate, cellulose, cellulose acetate propionate, cellulose acetate succinate, cellulose acetate phthalate, and/or hydroxypropyl methylcellulose acetate succinate.

50. The composition of claim 22, wherein the one or more acaloric phytonutrient(s) is a flavonoid, an iridoid, and/or an alkylresorcinol.

51. The composition of claim 50, wherein the one or more acaloric phytonutrient(s) is quercetin, kaempferol, myricetin, fisetin, rutin, isorhamnetin, naringenin, silybin, eriodictyol, apigenin, chrysin, delphinidin, betanin, cyanidin chloride, neohesperidin, epigallocatechin, diosmetin, baicalein, genistein, oleuropein, amarogentin, genipin, aucubin, catalpol, olivetol, cannabidiol, tetrahydrocannabinol, daidzein, pelargonidin, tangeritin, luteolin, wogonin, epicatechin, catechin, theaflavin, resveratrol, and/or hydroxytyrosol.

52. The composition of claim 13, wherein the at least one of the at least two shell(s) responsive to a pH less than 4.5 is characterized as buoyant, mucoadhesive, and/or retentive.

53. The composition of claim 7, wherein at least one shell of the at least two shells mitigates chemical and/or physical degradation of one or more lipid(s), protein(s), carbohydrate(s), and/or acaloric phytonutrient(s).

54. The composition of claim 53, wherein chemical degradation is characterized as a loss of greater than about 40% of the number and/or mass of one or more lipid(s), protein(s), carbohydrate(s), and/or acaloric phytonutrient(s) comprising one or more satiety modulating composition(s) upon a period of storage of at least 1 week, at least 1 month, at least 1 year, and/or at least 10 years at a temperature of at least 20° C., at least 40° C., and/or at least 80° C. in dry conditions.

55. The composition of claim 53, wherein chemical degradation is characterized as a loss of greater than about 40% of the number and/or mass of one or more lipid(s), protein(s), carbohydrate(s), and/or acaloric phytonutrient(s) comprising one or more satiety modulating composition(s) upon a period of storage of at least 1 week, at least 1 month, at least 1 year, and/or at least 10 years at a temperature of at least 20° C., at least 40° C., and/or at least 80° C. in high humidity conditions.

56. The composition of claim 53, wherein chemical degradation is characterized as a loss of greater than about 40% of the number and/or mass of one or more lipid(s), protein(s), carbohydrate(s), and/or acaloric phytonutrient(s) comprising one or more satiety modulating composition(s) upon a period of storage of at least 1 week, at least 1 month, at least 1 year, and/or at least 10 years at a temperature of at least 20° C., at least 40° C., and/or at least 80° C. in solution.

57. The composition of claim 1, wherein the water activity exhibited by the particle preparation is less than about 0.4, less than about 0.3, less than about 0.2, and/or less than about 0.1.

58. The composition of claim 1, wherein the total mass of one or more lipid(s), protein(s), carbohydrate(s), and acaloric phytonutrient(s) is at least about 0.1 g, at least about 0.5 g, at least about 1 g, at least about 5 g, at least about 10 g, at least about 50 g, and/or at least about 100 g.

59. The composition of claim 58, wherein the total mass of one or more lipid(s), protein(s), carbohydrate(s), and acaloric phytonutrient(s) is less than about 500 g.

60. The composition of claim 1, wherein the caloric content of one or more lipid(s), protein(s), and carbohydrate(s) is at least about 0 kcal, at least about 5 kcal, at least about 20 kcal, at least about 50 kcal, at least about 100 kcal, and/or at least about 250 kcal.

61. The composition of claim 60, wherein the caloric content of one or more lipid(s), protein(s), and carbohydrate(s) is less than about 500 kcal.

62. The composition of claim 1, wherein the particle preparation is amenable to incorporation into one or more food and/or beverage product(s).

63. The composition of claim 62, wherein the particle preparation is stable to size reduction techniques.

64. The composition of claim 63, wherein size reduction techniques comprise planetary milling, ball milling, burr milling, roller milling, media milling, impact milling, jet milling, high-pressure homogenization, cryo milling, hammer milling, conical milling, hand screening, or granulation/extrusion, extrusion, spray drying, lyophilization/milling, fluid bed agglomeration, spray congealing, high-shear granulation, tableting, pouring, roller compaction, crosslinking, prilling, spinning disc atomization, and/or combinations thereof.

65. The composition of claim 62, wherein the particle preparation is stable to homogenization techniques.

66. The composition of claim 62, wherein homogenization techniques comprise overhead stirrer, manual stirring, stir bar, high pressure homogenization, low pressure homogenization, sonication, ultrasonication, vortexing, or combinations thereof.

67. The composition of claim 62, wherein food and/or beverage product(s) comprise agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, tablet, yogurt, liquid supplement formulation, beer, seltzer, kefir, coffee, juice, liquid pharmaceutical formulation, milk, soda, sports drink, tea, water, liquor, and/or wine.

68. A non-therapeutic method of controlling satiety comprising:
a step of dispersing one or more nutraceutical particle preparations comprising a spatial arrangement of about 40% to about 75%, on a dry weight basis, of one or more lipid(s), about 10% to about 30%, on a dry weight basis, of one or more protein(s), about 10% to about 30%, on a dry weight basis, of one or more carbohydrate(s), and about 5% to about 15%, on a dry weight basis, of one or more acaloric phytonutrient(s) characterized by release of at least 90% of the one or more lipid(s), the one or more protein(s), the one or more carbohydrate(s), and/or the one or more acaloric phytonutrient(s) in an upper gastrointestinal region, a lower gastrointestinal region, or any combination thereof of one or more mammal(s) within a food and/or beverage matrix; and
a step of administering an effective dose of said preparation to one or more mammal(s).

69. The method of claim 68, wherein the food and/or beverage matrix is characterized as a food and/or beverage ingredient, an unfinished food and/or beverage product, a finished food and/or beverage product, and/or a food and/or beverage supplement.

70. The method of claim 69, wherein less than about 10% of one or more lipid(s), protein(s), carbohydrate(s), and/or acaloric phytonutrient(s) is observed in one or more food and/or beverage matrices.

71. The method of claim 70, wherein greater than about 90% of one or more lipid(s), protein(s), carbohydrate(s), and/or acaloric phytonutrient(s) is observed in the gastrointestinal tract of one or more mammal(s).

72. The method of claim 71, wherein greater than about 40% of one or more lipid(s), protein(s), carbohydrate(s), and/or acaloric phytonutrient(s) is observed in the upper gastrointestinal tract of one or more mammal(s).

73. The method of claim 71, wherein greater than about 50% of one or more lipid(s), protein(s), carbohydrate(s), and/or acaloric phytonutrient(s) is observed in the lower gastrointestinal tract of one or more mammal(s).

74. The method of claim 73, wherein one or more shell component(s) releases in the upper gastrointestinal tract of one or more mammal(s).

75. The method of claim 73, wherein one or more core component(s) releases in the lower gastrointestinal tract of one or more mammal(s).

76. The method of claim 69, wherein one or more particle preparation(s) is further characterized as a pill, a crystal, an emulsion, an oil, a dispersion, a tablet, a gel-cap, a solution, a gel, a powder, a sachet, a bar, a granule, a particle preparation, a core-shell preparation, a capsule, a jelly, a suspension, an elixir, a syrup, a food, and/or a beverage.

77. The method of claim 76, wherein one or more particle preparation(s) are administered orally, rectally, percutaneously, subcutaneously, and/or intravenously.

78. The method of claim 68, wherein an effective quantity of one or more lipid(s), protein(s), carbohydrate(s), and acaloric phytonutrient(s) is at least about 0.1 g, at least about 0.5 g, at least about 1 g, at least about 5 g, at least about 10 g, at least about 50 g, and/or at least about 100 g.

79. The method of claim 78, wherein the effective quantity of one or more lipid(s), protein(s), carbohydrate(s), and acaloric phytonutrient(s) is less than about 500 g.

80. The method of claim 68, wherein the effective quantity of one or more lipid(s), protein(s), and carbohydrate(s) is at least about 0 kcal, at least about 5 kcal, at least about 20 kcal, at least about 50 kcal, at least about 100 kcal, and/or at least about 250 kcal.

81. The method of claim 80, wherein the caloric content of one or more lipid(s), protein(s), and carbohydrate(s) is less than about 500 kcal.

82. The method of claim 68, wherein a step of administering a satiety modulating composition is measured as effective by subjective, self-reported grading by one or more mammal(s), relative to post-absorptive levels.

83. The method of claim 68, wherein a step of administering a satiety modulating composition is measured as effective by quantifying a change of at least 20% in at least one of leptin, GLP-1, GLP-2, motilin, gastrin, insulin, ghrelin, peptide yy, cholecystokinin, GIP, serotonin, histamine, oleoylethanolamide, palmitoylethanolamide, anandamide, and/or linolenoylethanolamide, their prehormones, their isoforms, their degradation products, and/or their full length and/or spliced transcripts in the serum of one or more mammal(s), relative to post-absorptive levels.

84. The method of claim 83, wherein satiety is controlled for at least about 6 hours, about 12 hours, about 16 hours, and/or about 24 hours.

85. A particle that is blend-able within a dairy product, the particle comprising a composition comprising:
eicosapentaenoic acid in a range from about 25% (w/w) to about 50% (w/w); and
α-tocopherol in a range from about 3% (w/w) to about 10% (w/w),
wherein the particle comprises a mean diameter (Dv50) of from about 300 μm to about 900 μm.

86. The particle of claim 85, comprising a composition comprising:
about 50% (w/w) eicosapentaenoic acid;
about 15% (w/w) carnauba wax;
about 15% (w/w) stearic acid;
about 15% (w/w) ethyl cellulose 100 cP; and
about 5% (w/w) α-tocopherol,
wherein the particle comprises a mean diameter (Dv₅₀) of from about 600 μm to about 700 m.

87. The particle of claim 85, comprising a composition comprising:
about 25% (w/w) eicosapentaenoic acid;
about 50% (w/w) EUDRAGUARD Protect;
about 15% (w/w) calcium carbonate; and
about 10% (w/w) α-tocopherol,
wherein the particle comprises a mean diameter (Dv₅₀) of from about 300 μm to about 400 m.

88. The particle of claim 85, comprising a composition comprising:
about 40% (w/w) eicosapentaenoic acid;
about 37% (w/w) whey protein isolate;
about 10% (w/w) sesame oil;
about 5% (w/w) diindolylmethane;
about 5% (w/w) sitosterol; and
about 3% (w/w) α-tocopherol,
wherein the particle comprises a mean diameter (Dv50) of from about 800 μm to about 900 m.

89. The particle of claim 86, comprising a mean diameter (Dv₅₀) of from about 650 μm to about 700 μm.

90. A mixture comprising:
about 90% (w/w) dairy product; and
about 10% (w/w) a particle comprising at least two satiety modulators.

91. The mixture of claim 90, wherein the particle comprises the particle of any of claims 86-88, and
wherein the dairy product comprises yogurt.

92. The mixture of claim 90, wherein the at least two satiety modulators comprise at least two of eicosapentaenoic acid, tocopherol, whey protein isolate, and diindolylmethane, and
wherein the dairy product comprises yogurt.

93. A coating method comprising:
solubilizing a first amount of encapsulant via at least one of melting and solvent-solubilization;
adding a second amount of nutrient payload to a coater, the coater comprising at least one of a pan coater, a fluidized bed coater and another coater;
applying at least one of fluidization, mixing, and rotation to the nutrient payload in the coater;
administering the melted or solubilized encapsulant to the mixed, rotated and/or fluidized payload via at least one of spraying, coating, and atomization thereby forming coated particles; and
adding at least one of anti-caking and flow-aid agents before, during and/or after the administering process.

94. The method of claim 93, further comprising:
collecting and mixing the coated particles; and
characterizing the coated particles via at least one of size analysis, shape analysis, release profile, and water activity.

95. A dissolution method comprising:
adding a satiety modulating composition to dissolution media;
initiating a dissolution assay comprising one or more desired conditions, the one or more desired conditions comprising at least one of a mixing condition, a specific temperature range, and specific pH range; and
collecting at least a portion of the dissolution media at various time points.

96. The method of claim 95, further comprising: performing analytical assays to determine release or dissolution characteristics,
wherein performing analytical assays comprises quantification of at least one of a payload and an encapsulant via at least one of HPLC, UV-vis, and spectroscopy.
